SEQUENCE LISTING (56 sequences)

SEQ ID NO: 1 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 254 aa, Neisseria meningitidis
CSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 262 aa, Neisseria meningitidis
CSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 5 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 6 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 7 - protein, 434 aa, synthetic construct
  REGION 1..434: note = Fusion protein of fHbp
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQRGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 8 - protein, 468 aa, Neisseria meningitidis
SPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQD

SEQ ID NO: 9 - protein, 644 aa, synthetic construct
  REGION 1..644: note = Fusion protein of NHBA
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 10 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 11 - protein, 261 aa, Neisseria meningitidis
CSSGSGSGGGGVAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 12 - protein, 260 aa, Neisseria meningitidis
CSSGGGGSGGGGVTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 13 - protein, 168 aa, Neisseria meningitidis
ATYKVDEYHANARFAIDHFNTSTNVGGFYGLTGSVEFDQAKRDGKIDITIPIANLQSGSQHFTDHLKSADIFDAAQYPDIRFVSTKFNFNGKKLVSVDGNLTMHGKTAPVKLKAEKFNCYQSPMEKTEVCGGDFSTTIDRTKWGMDYLVNVGMTKSVRIDIQIEAAKQ

SEQ ID NO: 14 - protein, 179 aa, Neisseria meningitidis
VSAVIGSAAVGAKSAVDRRTTGAQTDDNVMALRIETTARSYLRQNNQTKGYTPQISVVGYNRHLLLLGQVATEGEKQFVGQIARSEQAAEGVYNYITVASLPRTAGDIAGDTWNTSKVRATLLGISPATQARVKIVTYGNVTYVMGILTPEEQAQITQKVSTTVGVQKVITLYQNYVQR

SEQ ID NO: 15 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 16 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 17 - protein, 248 aa, synthetic construct
  REGION 1..248: note = MUTATED fhbp POLYPEPTIDE
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAAVETANGIHHIGLAAKQ

SEQ ID NO: 18 - protein, 248 aa, synthetic construct
  REGION 1..248: note = MUTATED fhbp POLYPEPTIDE
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 19 - protein, 263 aa, Neisseria meningitidis
CSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 20 - protein, 251 aa, Neisseria meningitidis
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 21 - protein, 251 aa, synthetic construct
  REGION 1..251: note = MUTATED fhbp POLYPEPTIDE
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYRLGIFGGQAQEVAGSAEVETANGIRHIGLAAKQ

SEQ ID NO: 22 - protein, 251 aa, synthetic construct
  REGION 1..251: note = MUTATED fhbp POLYPEPTIDE
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYRLGIFGGQAQEVAGSAEVETANGIRHIGLAAKQ

SEQ ID NO: 23 - protein, 251 aa, synthetic construct
  REGION 1..251: note = MUTATED fhbp POLYPEPTIDE
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYSLGIFGGQAQEVAGSAAVETANGIRHIGLAAKQ

SEQ ID NO: 24 - protein, 273 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 25 - protein, 254 aa, Neisseria meningitidis; identical to SEQ ID NO: 2

SEQ ID NO: 26 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 27 - protein, 281 aa, Neisseria meningitidis
MNRTAFCCLSLTTALILTACSSGGGGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 28 - protein, 262 aa, Neisseria meningitidis; identical to SEQ ID NO: 3

SEQ ID NO: 29 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 30 - protein, 247 aa, synthetic construct
  REGION 1..247: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 31 - protein, 250 aa, synthetic construct
  REGION 1..250: note = MUTATED fhbp POLYPEPTIDE
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 32 - protein, 757 aa, synthetic construct
  REGION 1..757: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAAVETANGIHHIGLAAKQ

SEQ ID NO: 33 - protein, 757 aa, synthetic construct
  REGION 1..757: note = MUTATED fhbp POLYPETIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 34 - protein, 760 aa, synthetic construct
  REGION 1..760: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYRLGIFGGQAQEVAGSAEVETANGIRHIGLAAKQ

SEQ ID NO: 35 - protein, 760 aa, synthetic construct
  REGION 1..760: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYRLGIFGGQAQEVAGSAEVETANGIRHIGLAAKQ

SEQ ID NO: 36 - protein, 760 aa, synthetic construct
  REGION 1..760: note = MUTATED fhbp POLYPEPTIDE
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYSLGIFGGQAQEVAGSAAVETANGIRHIGLAAKQ

SEQ ID NO: 37 - protein, 256 aa, synthetic construct
  REGION 1..256: note = fHbp polypeptide with His6 tag
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELGLAAKQLNVDLAAADIKPDGKRHAVISGSVLYNQAEKGSYSLGIFGGKAQEVAGSAEVKTVNGIRHLEHHHHHH

SEQ ID NO: 38 - protein, 256 aa, synthetic construct
  REGION 1..256: note = fHbp polypeptide with His6 tag
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQLEHHHHHH

SEQ ID NO: 39 - protein, 256 aa, synthetic construct
  REGION 1..256: note = Mutated fHbp polypeptide
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAEVETANGIHHIGLAAKQLEHHHHHH

SEQ ID NO: 40 - protein, 256 aa, synthetic construct
  REGION 1..256: note = Mutated fHbp polypeptide
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYRLGIFGGQAQEVAGSAEVETANGIHHIGLAAKQLEHHHHHH

SEQ ID NO: 41 - protein, 259 aa, synthetic construct
  REGION 1..259: note = fHbp polypeptide with His6 tag
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQLEHHHHHH

SEQ ID NO: 42 - protein, 259 aa, synthetic construct
  REGION 1..259: note = Mutated fHbp polypeptide with His6 tag
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAAKGSYSLGIFGGQAQEVAGSAEVETANGIRHIGLAAKQLEHHHHHH

SEQ ID NO: 43 - protein, 753 aa, synthetic construct
  REGION 1..753: note = fHbp fusion polypeptide
VAADIGAGLADALTAPLDHKDKSLQSLTLDQSVRKNEKLKLAAQGAEKTYGNGDSLNTGKLKNDKVSFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAAIKPDGKRHAVISGSVLYNQAEKGSYSLGIFGKAQEVAGSAEVKTVNGIRHIGLAAKQ

SEQ ID NO: 44 - protein, 753 aa, synthetic construct
  REGION 1..753: note = fHbp fusion polypeptide
VAADIGAGLADALTAPLDHKDKSLQSLTLDQVVRKNEKLKLAAQGAEKTYGNGDSLNTGKLKNDKVSFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAAIKPDGKRHAVISGSVLYNQAEKGSYSLGIFGKAQEVAGSAEVKTVNGIRHIGLAAKQ

SEQ ID NO: 45 - protein, 274 aa, Neisseria meningitidis
MNRTAFCCFSLTAALILTACSSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 46 - protein, 282 aa, Neisseria meningitidis
MNRTTFCCLSLTAALILTACSSGGGGSGGGGVAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 47 - protein, 254 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 48 - protein, 12 aa, synthetic construct
  REGION 1..12: note = N-terminal sequence
MGPDSDRLQQRR

SEQ ID NO: 49 - protein, 769 aa, synthetic construct
  REGION 1..769: note = MUTATED fhbp POLYPETIDE
MGPDSDRLQQRRVAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 50 - protein, 4 aa, synthetic construct
GGSG

SEQ ID NO: 51 - protein, 5 aa, synthetic construct
GGSGG

SEQ ID NO: 52 - protein, 5 aa, synthetic construct
GSGSG

SEQ ID NO: 53 - protein, 5 aa, synthetic construct
GSGGG

SEQ ID NO: 54 - protein, 5 aa, synthetic construct
GGGSG

SEQ ID NO: 55 - protein, 5 aa, synthetic construct
GSSSG

SEQ ID NO: 56 - protein, 6 aa, synthetic construct
GSGGGG

(19) United States
(12) Patent Application Publication
(10) Pub. No.: US 2026/0263578 A1
(43) Pub. Date: Sep. 10, 2026

(54) IMMUNOGENIC COMPOSITION

(71) Applicant: GLAXOSMITHKLINE BIOLOGICALS SA, Rixensart (BE)

(72) Inventors: Agnese MARCELLI, Siena (IT); Malte MEPPEN, Siena (IT); Daniela STRANGES, Siena (IT)

(73) Assignee: GLAXOSMITHKLINE BIOLOGICALS SA, Rixensart (BE)

(21) Appl. No.: 18/869,341

(22) PCT Filed: May 30, 2023
(86) PCT No.: PCT/EP2023/064439

§ 371 Date: Nov. 26, 2024

(30) Foreign Application Priority Data
Jun. 1, 2022 (GB) 2208093.1

(51) Int. Cl. A61K 39/095; A61K 9/50; A61K 39/00; A61P 37/04

(52) U.S. Cl. CPC A61K 39/095; A61K 9/5068; A61P 37/04; A61K 2039/555

(57) ABSTRACT

The present invention relates to an immunogenic composition against N. meningitidis serogroup B in liquid form, and to a reconstituted vaccine against N. meningitidis serogroups A, B, C, W135, and Y comprising the liquid composition. Kits and methods for the prevention and treatment of meningococcal infection and disease with the immunogenic composition or the reconstituted vaccine are also provided.

### TECHNICAL FIELD

This invention is in the field of vaccines. More in particular, it relates to an immunogenic composition against Neisseria meningitidis serogroup B in a liquid form, and to a reconstituted vaccine composition against N. meningitidis serogroups A, B, C, W, and Y obtained by reconstitution of an immunogenic composition against N. meningitidis serogroups A, C, W, and Y in a solid form into the liquid formulation. Kits and methods for immunizing a mammal against meningococcal infection and disease caused by the bacterial pathogen N. meningitidis, in particular of the infection and disease caused by serogroups A, B, C, W, and Y of this pathogen, with the above said reconstituted vaccine are also provided.

### BACKGROUND

Invasive meningococcal disease (IMD) is caused by the bacterial pathogen Neisseria meningitidis. Of the five serogroups mainly associated with IMD globally (Men A, B, C, W and Y), MenB is the predominant serogroup causing IMD in a number of regions, including Canada, the United States, Australia, New Zealand and Europe. MenB is a serious and often deadly disease, affecting mainly infants and young adults. It is easily mis-diagnosed, can kill within 24 hours of onset and can cause serious, life-long disabilities despite the administration of treatment.

There are currently two licensed vaccines that have been designed to immunize against serogroup B meningococcus: GSK's BEXSERO and Pfizer's TRUMENBA.

BEXSERO (also known generically, and referred to herein, as 4CMenB) contains a preparation of outer membrane vesicles (OMVs) from the epidemic strain of group B Meningococcal NZ98/254 together with five meningococcal antigens: Neisserial Heparin Binding protein A (NHBA), factor H binding protein (fHbp) variant 1.1, Neisserial adhesion protein A (NadA), and accessory proteins GNA1030 and GNA2091. Four of these antigens are present as fusion proteins (an NHBA-GNA1030 fusion protein and a GNA2091-fHbp fusion protein). 4CMenB is described in literature (for example, see Bai et al. (2011) Expert Opin Biol Ther. 11:969-85, Su & Snape (2011) Expert Rev Vaccines 10:575-88). The terms “BEXSERO” and “4CMenB” are used interchangeably herein.

TRUMENBA contains two lipidated MenB fHbp antigens (v1.55 and v3.45) adsorbed on aluminum phosphate.

fHbp (also known interchangeably in the art as genome-derived Neisseria antigen (GNA) 1870, LP2086 and protein ‘741’) binds to human factor H (hfH), which is a large (180 kDa) multi-domain soluble glycoprotein, consisting of 20 complement control protein (CCP) modules connected by short linker sequences. hfH circulates in human plasma and regulates the Alternative Pathway of the complement system. Functional binding of fHbp to hfH relies predominantly on CCP modules (or domains) 6-7 of hfH, and enhances the ability of the bacterium to resist complement-mediated killing. Therefore, expression of fHbp enables survival in ex vivo human blood and serum.

As different fHbp classification schemes have been proposed, a dedicated database is available with a unified fHbp nomenclature for the assignment of new sub-variants: (http)://neisseria.org/nm/typing/fhbp (also as (https)://pubmlst.org/neisseria/fHbp/).

fHbp has been classified into three (main) variants 1, 2 and 3, which were further divided into sub-variants fHbp-1.x, fHbp-2.x and fHbp-3.x, where x denotes the specific peptide sub-variant. In contrast to v2 and v3, fHbp v1 is highly heterogeneous and contains several subvariants. In a different nomenclature scheme, the sub/variants are grouped into subfamily A (corresponding to variants 2 and 3) and subfamily B (corresponding to variant 1) based on sequence diversity.

BEXSERO is predicted to provide broad coverage against MenB strains circulating worldwide (Medini D et al, Vaccine 2015; 33:2629-2636; Vogel U et al Lancet Infect Dis 2013; 13:416-425; Křížová et al., Epidemiol Mikrobiol Imunol 2014; 63:103-106; Tzanakaki G et al. BMC Microbiol 2014; 14:111; Wasko I et al Vaccine 2016; 34:510-515; 6. Simões M J et al PLoS ONE 12(5): e0176177; and Parikh S R et al. Lancet Infect Dis 2017; 17:754-62). Furthermore, following the introduction of BEXSERO into the UK national infant immunization programme in September 2015, data at 10 months showed 83% vaccine efficacy on all MenB strains after two doses (Parikh S R et al., Lancet 2016; 388:2775-82).

Recently, improved serogroup B meningococcus immunogenic compositions have been disclosed in WO 2020/030782, with mutated fHbp polypeptides and fusion proteins comprising these mutated polypeptides. These immunogenic compositions retain the efficacy of BEXSERO but also have an improved coverage against meningococcal strains carrying fHbp variants, in particular fHbp v2 and v3 variants, and strains carrying some v1 sub-variants.

In fact, bactericidal activity is variant specific; antibodies raised against one variant are not necessarily cross-protective against other variants, although some cross-reactivity has been described between fHbp v2 and v3 (Masignani V et al., J Exp Med 2003; 197:789-799). Antibodies raised against sub-variant fHbpv1.1, included in the 4CMenB vaccine, are highly cross-reactive with the most frequently occurring fHbp v1 sub-variants but are less cross-reactive with v1 sub-variants that are most distantly related to v1.1. Furthermore, antibodies raised against sub-variant fHbpv1.1 included in the 4CMenB vaccine are poorly cross-reactive with fHbp v2 and v3 (Brunelli B et al., Vaccine 2011; 29:1072-1081), while the improved vaccine comprising the mutated fHbp polypeptides provides a coverage against the variants too.

There are also vaccines that have been designed to immunize against serogroups A, C, W135 and Y of Neisseria meningitidis like GSK's MENVEO.

MENVEO comprises capsular saccharides of the meningococcal serogroups A, C, W135, and Y that are conjugated to a non-toxic mutant of diphtheria toxin as carrier protein, CRM197. The product on the market consists of a liquid formulation of the CRM197-conjugated MenCWY capsular saccharides for reconstitution of the solid, lyophilized MenA capsular saccharide conjugate.

The vaccine products marketed under the trade names MENACTRA, MENQUADFI and NIMENRIX also contain conjugated capsular saccharide antigens from each of serogroups Y, W135, C and A.

No vaccines are on the market up to date that have, in a single drug product, a coverage against meningococcal infection and disease caused by all main serogroups A, B, C, W135 and Y of N. meningitidis. Such a co-immunisation with a single vaccine composition in which the different immunogens are admixed offers to vaccinees the advantage of receiving a reduced number of injections, which can lead to the clinical advantage of increased compliance.

Besides this unmet need of having available such a product, there is also the need that this product represents an effective formulation, able to maintain the single antigens integrity and immunogenicity of the components and to guarantee at the same time the required safety, stability and easiness of use.

### SUMMARY OF THE INVENTION

The inventors have now found a liquid formulation comprising one or more tonicity modifying agents and an adsorbing agent, e.g. a compound containing aluminum, for an immunogenic composition against N. meningitidis serogroup B that enhances integrity and immunogenicity of antigens, maintaining at the same time at optimal levels all relevant quality requirements of the vaccine product, in particular pH and osmolality. These surprising results of enhanced adsorption of the MenB antigens onto the adsorbing agent in the formulation have been observed by the inventors even after reconstitution of a solid immunogenic composition of Men ACW135Y antigens with the present liquid formulation to obtain a Men ABCW135Y vaccine product.

Accordingly, a first aspect of this invention provides an immunogenic composition against N. meningitidis serogroup B, comprising a meningococcal NHBA antigen, a meningococcal NadA antigen, a meningococcal fHbp antigen, a meningococcal outer membrane vesicles (OMVs), and a fusion polypeptide of meningococcal fHbp polypeptides, in a liquid formulation comprising an adsorbing agent for the antigens and polypeptides and one or more pharmaceutically acceptable tonicity modifying agents.

A second aspect of this invention provides a reconstituted vaccine composition against N. meningitidis serogroups A, B, C, W135, and Y obtainable by reconstitution of an immunogenic composition against N. meningitidis serogroups A, C, W135, and Y, in a solid form (e.g. freeze-dried), with the above said immunogenic composition against N. meningitidis serogroup B in liquid form.

A third aspect of this invention provides a kit comprising (i) a first container comprising the above said immunogenic composition against N. meningitidis serogroup B in a liquid formulation; and (ii) a second container comprising an immunogenic composition against N. meningitidis serogroups A, C, W135, and Y in a solid form.

A fourth aspect of this invention provides a method for the preparation of a vaccine composition against N. meningitidis serogroups A, B, C, W135, and Y comprising the step of reconstituting an immunogenic composition in solid form against N. meningitidis serogroups A, C, W135, and Y with the above said immunogenic composition against N. meningitidis serogroup B in liquid form.

A fifth aspect of this invention is the above said immunogenic composition against N. meningitidis serogroup B in liquid form, or the reconstituted vaccine composition for use in immunizing a mammal, preferably a human, against meningococcal infection and disease caused by the bacterial pathogen N. meningitidis.

A sixth aspect of this invention provides a method of prevention or treatment of meningococcal infections or diseases caused by N. meningitidis, comprising administering to a subject in need thereof an immunogenic composition against N. meningitidis serogroup B in liquid form or a reconstituted vaccine of the invention.

A seventh aspect of this invention provides the use of the above said immunogenic composition against N. meningitidis serogroup B in liquid form, or of the reconstituted vaccine composition, in the manufacture of a medicament for the prevention or treatment of meningococcal infections or diseases caused by N. meningitidis.

### DESCRIPTION OF THE FIGURES

FIG. 1 illustrates chromatogram profiles overlapping of MenB formulations 2 (FORM 2) and 10 (FORM 10) according to the invention and for a BEXSERO-like formulation (Men B BEXSERO-like form) in comparison with standard point at a concentration of 8.0 μg/ml (STD 8.0 μg/ml).

FIG. 2 shows in form of histograms the amounts of GNA2091-fHbp (936-741) antigen not adsorbed onto Alum for different tested formulations of this invention and for BEXSERO before reconstitution, at time 0 (TO) and after 2 weeks (T2w) and 6 weeks (T6w) of storage at 2-8° C.

FIG. 3 shows in form of histograms the amounts of GNA2091-fHbp (936-741) antigen not adsorbed onto Alum for different tested formulations of this invention and for BEXSERO upon reconstitution with a lyophilised MenACWY product containing 2% of sucrose at time 0 (TO) and after 2 weeks (T2w) of storage at 2-8° C., as described in the following Example 4.

FIG. 4 shows in form of histograms the amounts of GNA2091-fHbp (936-741) antigen not adsorbed onto Alum for different tested formulations of this invention and for BEXSERO upon reconstitution with a lyophilised MenACWY product containing 3% of sucrose at time 0 (TO) and after 2 weeks (T2w) of storage at 2-8° C., as described in the following Example 4.

### DETAILED DESCRIPTION OF THE INVENTION

### General

The term “comprising” encompasses “including” as well as “consisting” e.g. a composition “comprising” X may consist exclusively of X or may include something additional e.g. X+Y. References to “comprising” (or “comprises”, etc.) may optionally be replaced by references to “consisting of” (or “consists of”, etc.). The term “consisting essentially of” limits the scope of a claim to the specified materials or steps “and those that do not materially affect the basic and novel characteristic(s)” of the claimed invention.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

Where the disclosure concerns an “epitope”, this epitope may be a B-cell epitope and/or a T-cell epitope, but will usually be a B-cell epitope. Such epitopes can be identified empirically (e.g. using PEPSCAN (e.g. see Geysen et al. (1984) PNAS USA 81:3998-4002 and Carter (1994) Methods Mol Biol 36:207-23) or similar methods), or they can be predicted (e.g. using the Jameson-Wolf antigenic index (Jameson, B A et al. 1988, CABIOS 4(1):181-186), matrix-based approaches (Raddrizzani & Hammer (2000) Brief Bioinform 1(2):179-89), MAPITOPE (Bublil et al. (2007) Proteins 68(1):294-304), TEPITOPE (De Lalla et al. (1999) J. Immunol. 163:1725-29 and Kwok et al. (2001) Trends Immunol 22:583-88), neural networks (Brusic et al. (1998) Bioinformatics 14(2):121-30), OptiMer & EpiMer (Meister et al. (1995) Vaccine 13(6):581-91 and Roberts et al. (1996) AIDSRes Hum Retroviruses 12(7):593-610), ADEPT (Maksyutov & Zagrebelnaya (1993) Comput Appl Biosci 9(3):291-7), Tsites (Feller & de la Cruz (1991) Nature 349(6311):720-1), hydrophilicity (Hopp (1993) Peptide Research 6:183-190), or antigenic index (Welling et al. (1985) FEBS Lett. 188:215-218)). Epitopes are the parts of an antigen that are recognized by and bind to the antigen binding sites of antibodies or T-cell receptors, and they may also be referred to as “antigenic determinants”.

The term “suspension” means a mixture in which solid particles are dispersed throughout a liquid, including throughout a liquid composition.

As used herein, references to “percentage sequence identity” between a query amino acid sequence and a subject amino acid sequence are understood to refer to the value of identity that is calculated using a suitable algorithm or software program known in the art to perform pairwise sequence alignment.

A query amino acid sequence may be described by an amino acid sequence identified in one or more claims herein. The query sequence may be 100% identical to the subject sequence, or it may include up to a certain integer number of amino acid alterations (e.g. point mutations, substitutions, deletions, insertions etc.) as compared to the subject sequence, such that the % identity is less than 100%. For example, the query sequence is at least 80, 85, 90, 95, 96, 97, 98, or 99% identical to the subject sequence.

Preferred alignment tools used to perform alignment and calculate percentage (%) sequence identity are local alignment tools, such as the Basic Local Alignment Search Tool (BLAST) algorithms. Software for performing BLAST analyses is publicly available through the National Centre for Biotechnology Information (www.ncbi.nlm.nih.gov). Alignment may be determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489. Other preferred alignment tools are Water (EMBOSS) and Marcher (EMBOSS). Alternatively, preferred alignment tools used to perform alignment and calculate percentage (%) sequence identity are best fit alignment tools, such as GENEPAST, also known as KERR algorithm.

In order to calculate percent identity, the query and subject sequences may be compared and aligned for maximum correspondence over a designated region (e.g. a region of at least about 40, 45, 50, 55, 60, 65 or more amino acids in length, and can be up to the full length of the subject amino acid sequence).

Said designated region must include the region of the query sequence comprising any specified point mutations in the amino acid sequence. Alternatively, percentage sequence identity may be calculated over the “full length” of the subject sequence. Any N-terminal or C-terminal amino acid stretches that may be present in the query sequence, such as signal peptides or leader peptide or C-terminal or N-terminal tags, should excluded from the alignment.

The term “fragment” in reference to polypeptide sequences means that the polypeptide is a fraction of a full-length protein. As used herein, a fragment of a mutant polypeptide also comprises the mutation(s). Fragments may possess qualitative biological activity in common with the full-length protein, for example, an “immunogenic fragment” contains or encodes one or more epitopes, such as immunodominant epitopes, that allows the same or similar immune response to be raised to the fragment as is raised to the full-length sequence.

Polypeptide fragments generally have an amino (N) terminus portion and/or carboxy (C) terminus portion deleted as compared to the native protein, but wherein the remaining amino acid sequence of the fragment is identical to the amino acid sequence of the native protein. Polypeptide fragments may contain, for example: about 8, 10, 12, 14, 16, 18, 20, 22, 24, 26, 28, 30, 32, 24, 26, 28, 40, 45, 50, 55, 60, 70, 80, 90, 100, 150, 200, 240, 241, 242, 243, 244, 245, 246, 247, 248, 249, 250, 251, 252, 253, 254, 255, 256, 257, 258, 259, 260, 261, 262 contiguous amino acids, including all integers in between, of a reference polypeptide sequence, for example between 50 and 260, 50 and 255, 50 and 250, 50 and 200, 50 and 150 contiguous amino acids of a reference polypeptide sequence. The term fragment explicitly excludes full length fHbp polypeptides and mature lipoproteins thereof.

After serogroup, meningococcal classification includes serotype, serosubtype and then immunotype, and the standard nomenclature lists serogroup, serotype, serosubtype, and immunotype, each separated by a colon e.g. B:4: P1.15:L3,7,9. Within serogroup B, some lineages cause disease often (hyperinvasive), some lineages cause more severe forms of disease than others (hypervirulent), and others rarely cause disease at all. Seven hypervirulent lineages are recognised, namely subgroups I, III and IV-1, ET-5 complex, ET-37 complex, A4 cluster and lineage 3. These have been defined by multilocus enzyme electrophoresis (MLEE), but multilocus sequence typing (MLST) has also been used to classify meningococci. The four main hypervirulent clusters are ST32, ST44, ST8 and ST11 complexes.

References herein to “enhanced stability” or “higher stability” or “increased stability” mean that the mutant polypeptides disclosed herein have a higher relative thermostability (in kcal/mol) as compared to a non-mutant (wild-type) polypeptide under the same experimental conditions. The stability enhancement can be assessed using differential scanning calorimetry (DSC), for example as discussed in Bruylants et al. (Differential Scanning Calorimetry in Life Sciences: Thermodynamics, Stability, Molecular Recognition and Application in Drug Design, 2005 Curr. Med. Chem. 12: 2011-2020) and Calorimetry Sciences Corporation's “Characterizing Protein stability by DSC” (Life Sciences Application Note, Doc. No. 2021102136 February 2006) or by differential scanning fluorimetry (DSF). An increase in stability may be characterized as an at least about 5° C. increase in thermal transition midpoint (Tₘ), as assessed by DSC or DSF. See, for example, Thomas et al., Effect of single-point mutations on the stability and immunogenicity of a recombinant ricin A chain subunit vaccine antigen, 2013 Hum. Vaccin. Immunother. 9(4): 744-752.

### Meningococcus Serogroup B

It is a first subject of this invention an immunogenic composition against N. meningitidis serogroup B, comprising two or more antigens selected from: a meningococcal NHBA antigen, a meningococcal NadA antigen, a meningococcal fHbp antigen, a meningococcal outer membrane vesicles (OMVs), and a fusion polypeptide of meningococcal fHbp polypeptides, which is in the form of a liquid formulation comprising an adsorbing agent for the antigens and polypeptides and one or more pharmaceutically acceptable tonicity modifying agents.

The MenB composition in a liquid formulation may comprise one or more antigens, as disclosed for instance in WO2004/032958, WO2016/008960, and WO2020/030782, all of which are incorporated herewith by reference.

This Men B composition in liquid formulation may comprise for instance two or more antigens selected from: a meningococcal NHBA antigen, a meningococcal NadA antigen, a meningococcal fHbp antigen and meningococcal outer membrane vesicles (OMVs). The Men B composition in liquid formulation may comprise for instance a meningococcal NadA antigen, a meningococcal fHbp antigen and meningococcal outer membrane vesicles (OMVs). The Men B composition in liquid formulation may comprise for instance a meningococcal NHBA antigen, a meningococcal NadA antigen, a meningococcal fHbp antigen and meningococcal outer membrane vesicles (OMVs).

The Men B composition in liquid formulation may comprise for instance two or more fHbp antigens. The Men B composition in liquid formulation may comprise for instance two or more meningococcal fHbp antigens. The Men B composition in liquid formulation may comprise for instance a meningococcal NadA antigen, two or more meningococcal fHbp antigens and meningococcal outer membrane vesicles (OMVs). The Men B composition in liquid formulation may comprise for instance a meningococcal NHBA antigen, a meningococcal NadA antigen, two or more meningococcal fHbp antigens and meningococcal outer membrane vesicles (OMVs).

In an aspect of this invention, the liquid formulation of Men B antigens may further comprise a fusion polypeptide of meningococcal fHbp polypeptides. The Men B composition in liquid formulation may comprise for instance a meningococcal NHBA antigen, a meningococcal NadA antigen, a fusion polypeptide of meningococcal fHbp polypeptides and meningococcal outer membrane vesicles (OMVs).

In a preferred embodiment, the meningococcal NHBA antigen and the meningococcal fHbp antigen of this immunogenic composition in liquid form are fusion proteins with meningococcal accessory proteins, e.g. with GNA1030 and GNA2091. Most preferably, the meningococcal NHBA antigen and the meningococcal fHbp antigen of this immunogenic composition are respectively an NHBA-GNA1030 fusion protein and a GNA2091-fHbp fusion protein.

Where the present immunogenic composition against N. meningitidis serogroup B comprises OMVs, these OMVs can be any proteoliposomic vesicle obtained by disruption of or blebbing from a meningococcal outer membrane to form vesicles therefrom that retain antigens from the outer membrane. Thus, this term includes, for instance, OMVs (sometimes referred to as ‘blebs’), microvesicles (MVs), ‘native OMVs’ (‘NOMVs’) extracted from cells using detergent-free methods, and detergent-extracted OMVs (dOMVs), such as OMVs extracted from cells using deoxycholate treatment.

The mass of OMVs is measured as the amount of total protein.

Preferred meningococcal OMVs comprise a PorA serotype 1.4. Preferably, the OMVs comprise a PorA variable region epitope 1.7-2 (VR1) and/or 1.4 (VR2). OMVs comprising both of these epitopes are more preferred (i.e. P1.7-2,4). OMVs obtained from strain NZ98/254 are particularly preferred.

In a most preferred embodiment, the immunogenic composition against N. meningitidis serogroup B in liquid form contains, as the OMV antigen, a preparation of OMV from the epidemic strain of group B Meningococcal NZ98/254, B:4:P1.7b,4.

In a further preferred embodiment, the immunogenic composition against N. meningitidis serogroup B of the invention comprises, besides the OMV antigen and the fusion fHbp polypeptide, five meningococcal antigens: NHBA (287; subvariant 1.2), fHbp (741; subvariant 1.1), NadA (961; subvariant 3.1), GNA1030 (953) and GNA2091 (936). Four of these antigens are present as fusion proteins (an NHBA-GNA1030 fusion protein (287-953) and a GNA2091-fHbp (936-741) fusion protein).

In one embodiment, the immunogenic composition of the invention against N. meningitidis serogroup B comprises the complete vaccine product 4CMenB, marketed under the trade name BEXSERO.

The polypeptides (or a subset thereof, e.g. non-OMV or soluble polypeptides) in a composition may be present at substantially equal masses i.e. the mass of each of them is within +5% of the mean mass of all the polypeptides in the composition (or the mean mass of the selected subset of polypeptides). For example, where the composition includes NHBA, fHbp and NadA, they may be present at substantially equal masses, e.g. at a mass ratio of a:b:c, where each of a, b & c is between 0.95 and 1.05.

### NHBA (Neisserial Heparin Binding Antigen)

NHBA was included in the published genome sequence for meningococcal serogroup B strain MC58 (Tettelin et al. (2000) Science 287:1809-1815) as gene NMB2132 (GenBank accession number GI:7227388; SEQ ID NO: 4 herein).

References to NHBA herein include truncated variants of NHBA, wherein the N-terminus of the wild-type NHBA polypeptide sequence has been deleted up to and including its poly-glycine sequence (i.e. deletion of residues 1 to 24 in meningococcal strain MC58 (SEQ ID No. 4)). The resulting truncated variant is sometimes distinguished herein by the use of a ‘ΔG’ prefix. This deletion can enhance expression. The ‘ΔG’ variant of meningococcal NHBA is referred to herein as SEQ ID NO. 8.

Preferred NHBA antigens for use with the invention comprise an amino acid sequence: (a) having 70% or more identity (e.g. 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘η’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 4. Particularly preferred NHBA antigens for use with the invention comprise the amino acid sequence of SEQ ID NO: 8.

A polypeptide including a neisserial NHBA antigen sequence can include that sequence alone, or it can be a fusion protein. One useful fusion partner for a NHBA sequence is the GNA1030 (953) polypeptide, which will normally be downstream of the NHBA sequence. Thus, the NHBA antigen can be present in a composition of the invention as a NHBA-GNA1030 fusion (e.g. SEQ ID NO: 9).

### NadA (Neisserial Adhesin A)

The NadA antigen was included in the published genome sequence for meningococcal serogroup B strain MC58 (Tettelin et al. (2000) Science 287:1809-1815) as gene NMB 1994 (GenBank accession number GI:7227256; SEQ ID NO: 5 herein).

Preferred NadA antigens for use with the invention comprise an amino acid sequence: (a) having 70% or more identity (e.g. 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 5. SEQ ID NO: 10 is one such fragment. Particularly preferred NadA antigens for use according to the invention comprise SEQ ID NO: 10.

fHbp (Factor H Binding Protein)

The fHbp antigen has been characterized in detail. It has also been known as protein ‘741’ (SEQ IDs 2535 & 2536 in WO99/57280), ‘NMB 1870’, ‘GNA1870’ (e.g. Masignani V. et al. (2003) J. Exp. Med. 197:789-799), ‘P2086’, ‘LP2086’ or ORF2086′ (e.g. WO03/063766). It is expressed across many meningococcal serogroups, in which it is a lipoprotein.

The fHbp antigen falls into three distinct variants (see WO2004/048404) and it has been found that for meningococci, serum raised against a given family is bactericidal within the same family but is not active against strains which express one of the other two families i. e. there is intra-family cross-protection, but not inter-family cross-protection. The invention can use a single fHbp variant, but to provide broader coverage a composition can usefully include a fHbp from two or three of the variants.

Where a composition comprises a single fHbp antigen it may include one of the following:
(a) a first polypeptide comprising a first amino acid sequence, where the first amino acid sequence comprises an amino acid sequence (i) having at least a % sequence identity to SEQ ID NO:1 (strain MC58) and/or (ii) consisting of a fragment of at least x contiguous amino acids from SEQ ID NO: 1;
(b) a second polypeptide, comprising a second amino acid sequence, where the second amino acid sequence comprises an amino acid sequence (i) having at least b % sequence identity to SEQ ID NO: 2 (strain 961-5945) and/or (ii) consisting of a fragment of at least y contiguous amino acids from SEQ ID NO: 2;
(c) a third polypeptide, comprising a third amino acid sequence, where the third amino acid sequence comprises an amino acid sequence (i) having at least c % sequence identity to SEQ ID NO: 3 (strain M1239) and/or (ii) consisting of a fragment of at least z contiguous amino acids from SEQ ID NO: 3.

Where a composition comprises two different meningococcal fHbp antigens, it may include a combination of: (i) a first and second polypeptide as defined above; (ii) a first and third polypeptide as defined above; or (iii) a second and third polypeptide as defined above. A combination of a first and third polypeptide is preferred. If a single fHbp antigen is used, it is preferred that it is a first or a third polypeptide as described above.

In other embodiments a composition comprises three different meningococcal fHbp antigens, with first, second and third polypeptides as defined above. Where a composition comprises two or three different meningococcal fHbp antigens, although these may share some sequences in common, the first, second and third polypeptides have different fHbp amino acid sequences.

In some embodiments the fragment of at least x contiguous amino acids from SEQ ID NO: 1 is not also present within SEQ ID NO: 2 or within SEQ ID NO: 3. Similarly, the fragment of at least y contiguous amino acids from SEQ ID NO: 2 might not also be present within SEQ ID NO: 1 or within SEQ ID NO: 3. Similarly, the fragment of at least z contiguous amino acids from SEQ ID NO: 3 might not also be present within SEQ ID NO: 1 or within SEQ ID NO: 2. In some embodiments, when said fragment from one of SEQ ID NOs: 1 to 3 is aligned as a contiguous sequence against the other two SEQ ID NOs, the identity between the fragment and each of the other two SEQ ID NOs is less than 75% e.g. less than 70%, less than 65%), less than 60%>, etc.

The value of a is at least 80 e.g. 82, 84, 86, 88, 90, 92, 94, 95, 96, 97, 98, 99 or more. The value of b is at least 80 e.g. 82, 84, 86, 88, 90, 92, 94, 95, 96, 97, 98, 99 or more. The value of c is at least 80 e.g. 82, 84, 86, 88, 90, 92, 94, 95, 96, 97, 98, 99 or more. The values of a, b and c may be the same or different. In some embodiments, a b and c are identical.

The value of x is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of y is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of z is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The values of x, y and z may be the same or different. In some embodiments, x y and z are identical.

Fragments preferably comprise an epitope from the respective SEQ ID NO: sequence. Other useful fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of the respective SEQ ID NO: while retaining at least one epitope thereof.

Amino acid sequences used with the invention may, compared to SEQ ID NOs: 1, 2 or 3, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may have one or more (e.g. 1, 2, 3, 4, 5, 6, 7, 8, 9, 10, etc.) single amino acid deletions relative to a reference sequence. The polypeptides may also include one or more (e.g. 1, 2, 3, 4, 5, 6, 7, 8, 9, 10, etc.) insertions (e.g. each of 1, 2, 3, 4 or 5 amino acids) relative to a reference sequence.

A useful first amino acid sequence has at least 85% identity (e.g. >90%, 95% or 100%) to SEQ ID NO: 1. Another useful first amino acid sequence has at least 95% identity (e.g. >98% or 100%) to SEQ ID NO: 12. Preferred fHbp sequences for use according to the invention comprise SEQ ID NO:6. A useful third amino acid sequence has at least 85% identity (e.g. >90%, 95% or 100%) to SEQ ID NO: 3. Another useful third amino acid sequence has at least 95% identity (e.g. >98% or 100%) to SEQ ID NO: 11.

Combinations comprising a mixture of first and third sequences based around SEQ ID NOs: 11 and 12 (or their close variants) are particularly useful. Thus, a composition may comprise a polypeptide comprising amino acid sequence SEQ ID NO: 11 and a further polypeptide comprising amino acid sequence SEQ ID NO: 12. fHbp antigens used with the invention can be lipidated e.g. at a N-terminus cysteine residue. In other embodiments they will not be lipidated, and may include amino acid sequences upstream of the natural mature N-terminal cysteine. SEQ ID NOs: 1-3 and 11-12 begin with the cysteine from the natural N-terminus of the relevant mature fHbp polypeptides. For lipidated fHBPs, lipids attached to cysteines will usually include palmitoyl residues e.g. as tripalmitoyl-S-glyceryl-cysteine (Pam3Cys), dipalmitoyl-S-glyceryl cysteine (Pam2Cys), N-acetyl (dipalmitoyl-S-glyceryl cysteine), etc.

A polypeptide including the fHbp antigen sequence can include that sequence alone, or it can be a fusion polypeptide. One useful fusion partner for a fHbp sequence is the GNA2091 polypeptide, which will normally be upstream of the fHbp sequence. Thus, the fHbp antigen can be present in a composition of the invention as a GNA2091-fHbp fusion e.g. SEQ ID NO: 7.

Compositions used with the invention may also include an fHbp fusion protein comprising 2 or 3 of the first, second and third amino acid sequences defined at (a) to (c) above.

Compositions used with the invention may also include an fHbp protein that is mutated relative to SEQ ID NO: 1, 2 or 3 (fHbp variant 1, 2 or 3 respectively) to decrease binding to human factor H (fH). Suitable mutations are disclosed in Rossi et al. (2013) Vaccine 31:5451-7.

### GNA1030 Antigens

‘GNA1030’ protein from meningococcus serogroup B is disclosed as ‘953’ in WO99/57280 (SEQ IDs 2917 & 2918 therein) and as ‘NMB1030’ in Tettelin et al. (2000) Science 287:1809-1815 (see also GenBank accession number GI:7226269). The corresponding protein in serogroup A (see Parkhill et al (2000) Nature 404:502-506) has GenBank accession number 7380108.

When used according to the present invention, GNA1030 protein may take various forms. Preferred forms of GNA1030 are truncation or deletion variants, such as those disclosed in WO01/64920, WO01/64922, and WO03/020756. In particular, the N-terminus leader peptide of GNA1030 may be deleted (i.e. deletion of residues 1 to 19 for strain MC58 [SEQ ID NO: 13]) to give GNA1030(NL)

Preferred GNA1030 sequences have 50% or more identity (e.g. 60%, 70%, 80%, 90%, 95%, 99% or more) to SEQ ID NO: 13. This includes GNA1030 variants (e.g. allelic variants, homologs, orthologs, paralogs, mutants, etc.).

Other preferred GNA1030 sequences comprise at least n consecutive amino acids from SEQ ID NO: 13, wherein n is 7 or more (eg. 8, 10, 12, 14, 16, 18, 20, 25, 30, 35, 40, 50, 60, 70, 80, 90, 100, 150, 200, 250 or more). Preferred fragments comprise an epitope from GNA1030, in which case detection of the epitope in a pathogen of interest may be performed using a monoclonal antibody to the epitope. Other preferred fragments lack one or more amino acids (e.g. 1, 2, 3, 4, 5, 6, 7, 8, 9, 10, 15, 20, 25 or more) from the C-terminus and/or the N-terminus of SEQ ID NO: 13.

### GNA2091 Antigens

‘GNA2091’ protein from meningococcus serogroup B is disclosed as protein 936 in WO99/57280 (SEQ IDs 2883 & 2884) and as ‘NMB2091’ in Tettelin et al. (2000) Science 287:1809-1815 (see also GenBank accession number GI: 7227353). The corresponding gene in serogroup A (see Parkhill et al (2000) Nature 404:502-506) has GenBank accession number 7379093.

When used according to the present invention, GNA2091 protein may take various forms. Preferred forms of GNA2091 are truncation or deletion variants, such as those disclosed in WO01/64920, WO01/64922, and WO03/020756. In particular, the N-terminus leader peptide of GNA2091 may be deleted (i.e. deletion of residues 1 to 23 for strain MC58 [SEQ ID NO: 14]) to give GNA2091^{(NL)}.

Preferred GNA2091 sequences have 50% or more identity (e.g. 60%, 70%, 80%, 90%, 95%, 99% or more) to SEQ ID NO: 14. This includes variants (e.g. allelic variants, homologs, orthologs, paralogs, mutants etc).

Other preferred GNA2091 sequences comprise at least n consecutive amino acids from SEQ ID NO: 14, wherein n is 7 or more (eg. 8, 10, 12, 14, 16, 18, 20, 25, 30, 35, 40, 50, 60, 70, 80, 90, 100, 150, 200, 250 or more). Preferred fragments comprise an epitope from GNA2091, in which case detection of the epitope in a pathogen of interest may be performed using a monoclonal antibody to the epitope. Other preferred fragments lack one or more amino acids (e.g. 1, 2, 3, 4, 5, 6, 7, 8, 9, 10, 15, 20, 25 or more) from the C-terminus and/or the N-terminus of SEQ ID NO: 14.

Mutant fHbp Polypeptides

In another embodiment of this invention, the immunogenic composition against N. meningitidis serogroup B in liquid formulation comprises the antigens in the immunogenic composition disclosed in WO 2020/030782, which is incorporated here by reference, including the fusion polypeptides comprising mutated fHbp polypeptides. These immunogenic compositions have been found to retain the efficacy of the 4CMenB compositions (BEXSERO) but also have an improved coverage against meningococcal strains carrying fHbp variants thanks to the inclusion of the fusion fHbp polypeptides.

The lipoprotein factor H binding protein (fHbp) is expressed on the surface of all MenB strains. fHbp binds to the human complement regulatory protein factor H (hfH), forming a complex that protects the bacteria from complement-mediated killing and providing a survival mechanism for N. meningitidis in the human bloodstream. Antibodies against fHbp have a dual role: they are bactericidal perse, and by preventing binding to hfH they render strains more susceptible to bacterial killing. Reducing or abolishing the ability of fHbp to bind to hfH increases the immunogenicity of the fHbp antigen by preventing the formation of protective complexes between fHbp and hfH which have potential to mask fHbp epitopes and prevent antibody binding.

fHbp exists in three different genetic and immunogenic variants (v1, v2 and v3), with many subvariants. The majority of MenB strains that are not covered by BEXSERO express fHbp in v2, v3 or v1 subvariants distantly related to var1.1 (var1.1 being the fHbp antigen that is included in BEXSERO).

WO2020/030782 discloses mutated fHbp variant 1 (v1) polypeptides that are immunogenic and can be combined with existing meningococcal vaccines to provide improved N. meningitidis strain coverage. In particular, these v1 polypeptides are subvariants of fHbp variant 1 that are genetically diverse compared with the fHbp v1.1 antigen included in BEXSERO.

Furthermore, the v1 polypeptides disclosed in WO2020/030782 are mutated in order to reduce binding to hfH compared with the corresponding wildtype v1 polypeptide. In contrast, the fHbp v1.1 antigen included in BEXSERO, and the fHp v1.55 and v3.45 antigens included in TRUMENBA, do bind to hfH.

The v1 polypeptides disclosed in WO2020/030782 can be provided alone or as a component of a fusion protein, together with mutant forms of fHbp variants 2 and 3, which have been modified to improve stability and also to reduce fHbp binding. By providing a single fusion protein comprising these v2 and v3 antigens, together with a v1 antigen of the invention, the inventors improve strain coverage. For clarity, neither of the v2 and v3 antigens are present in, e.g., BEXSERO. The presence of v2 and v3 antigens within the fusion proteins of the present invention improves strain coverage as compared to, e.g., BEXSERO.

The v1 polypeptides and fusion proteins are preferably used in combination with a meningococcal NHBA antigen, a meningococcal NadA antigen, a meningococcal fHbp antigen, and a meningococcal outer membrane vesicle (e.g., in combination with the BEXSERO composition as described above), to provide a combined immunogenic composition having increased immunogenicity (due to the addition/inclusion of non-binding forms of fHbp variants) and increased N. meningitidis serotype B strain coverage (due to the addition of new fHbp variants/subvariants), compared with BEXSERO alone.

The inventors of WO2020/030782 identified residues within the fHbp v1.13 sequence that can be modified to reduce binding to hfH. Such mutant v1.13 meningococcal fHbp polypeptides are referred to herein as non-binding (NB) mutants. The inventors also identified combinations of mutations in the v1.13 sequence that are particularly useful to reduce binding to hfH. fHbp v1.13 is also known in the art as fHbp variant B09.

The mature wild type fHbp v1.13 lipoprotein from strain M982 (GenBank Accession No. AAR84475.1) has the following amino acid sequence, with an N-terminal poly-glycine signal sequence being underlined:

(SEQ ID NO: 15)
CSSGGGGVAADIGAGLADALTAPLDHKDKGLQSLTLDQSVRKNEKLK
LAAQGAEKTYGNGDSLNTGKLKNDKVSRFDFIRQIEVDGKLITLESG
EFQVYKQSHSALTALQTEQVQDSEDSGKMVAKRQFRIGDIAGEHTSF
DKLPKGGSATYRGTAFGSDDAGGKLTYTIDFAAKQGHGKIEHLKSPE
LNVELATAYIKPDEKRHAVISGSVLYNQDEKGSYSLGIFGGQAQEVA
GSAEVETANGIHHIGLAAKQ

The mature v1.13 lipoprotein differs from the full-length wild-type sequence in that the full-length polypeptide has an additional 19 residue N-terminal leader sequence, which is cleaved from the mature polypeptide. Thus, full-length wild-type fHbp v1.13 has the following amino acid sequence of SEQ ID NO: 45 (with the N-terminal leader sequence shown in bold font):

(SEQ ID NO: 45)
MNRTAFCCFSLTAALILTACSSGGGGVAADIGAGLADALTAPLDHKD
KGLQSLTLDQSVRKNEKLKLAAQGAEKTYGNGDSLNTGKLKNDKVSR
FDFIRQIEVDGKLITLESGEFQVYKQSHSALTALQTEQVQDSEDSGK
MVAKRQFRIGDIAGEHTSFDKLPKGGSATYRGTAFGSDDAGGKLTYT
IDFAAKQGHGKIEHLKSPELNVELATAYIKPDEKRHAVISGSVLYNQ
DEKGSYSLGIFGGQAQEVAGSAEVETANGIHHIGLAAKQ

The ΔG form of the mature v1.13 lipoprotein lacks the N-terminal poly-glycine sequence of the mature polypeptide, i.e. it lacks the first 7 amino acids of SEQ ID NO: 15, and it lacks the first 26 amino acids of SEQ ID NO: 45:

(SEQ ID NO: 16)
VAADIGAGLADALTAPLDHKDKGLQSLTLDQSVRKNEKLKLAAQGAE
KTYGNGDSLNTGKLKNDKVSRFDFIRQIEVDGKLITLESGEFQVYKQ
SHSALTALQTEQVQDSEDSGKMVAKRQFRIGDIAGEHTSFDKLPKGG
SATYRGTAFGSDDAGGKLTYTIDFAAKQGHGKIEHLKSPELNVELAT
AYIKPDEKRHAVISGSVLYNQDEKGSYSLGIFGGQAQEVAGSAEVET
ANGIHHIGLAAKQ

In one embodiment, the serogroup B antigenic component of the immunogenic composition of this invention comprises a mutant v1.13 meningococcal fHbp polypeptide comprising an amino acid sequence having at least k % sequence identity to SEQ ID NO: 16, with the proviso that the amino acid sequence of said mutant v1.13 meningococcal fHbp polypeptide includes a substitution mutation, i.e. a point mutation, at one or more of residues E211, S216 or E232 of SEQ ID NO: 16.

The value of k may be selected from 80, 85, 86, 87, 88, 89, 90, 91, 92, 93, 94, 95, 96, 97, 98, 99 or 100. k % is preferably 80% (i.e. the mutant fHbp v1.13 amino acid sequence has at least 80% identity to SEQ ID NO: 16) and is more preferably 85%, more preferably 90% and more preferably 95%. Most preferably, the mutant fHbp v1.13 amino acid sequence has at least 97%, at least 98% or at least 99% identity to SEQ ID NO: 16.

Thus, the present invention provides a reconstituted vaccine comprising a mutant v1.13 meningococcal fHbp polypeptide comprising an amino acid sequence having at least 80% sequence identity to SEQ ID NO: 16 wherein the amino acid sequence includes a substitution mutation, i.e. a point mutation, at one or more of residues S216, E211 or E232 of SEQ ID NO: 16.

Preferably, the amino acid sequence differs from SEQ ID NO: 16 by at least one or more of the substitutions E211A, S216R or E232A. More preferably, the amino acid sequence comprises substitutions at multiple residues selected from the following (i) E211A and E232A, or (ii) E211A and S216R. More preferably, the amino acid sequence comprises substitutions at residues E211A and S216R, relative to SEQ ID NO. 16.

Without wishing to be bound by theory, the substitution of glutamic acid (E) for alanine (A) at residue 211 of SEQ ID NO. 16 removes a negatively charged residue that is involved in hfH recruitment, thus contributing to the abrogation of fH binding. The substitution of arginine (R) for serine (S) at residue 216 of SEQ ID NO. 16 replaces the wildtype amino acid with a corresponding residue from N. gonorrhoeae, which does not bind hfH.

In preferred embodiments, a mutant v1.13 polypeptide of the invention has the amino acid sequence of SEQ ID NO: 17 (v1.13 ΔG E211A/E232A) or SEQ ID NO: 18 (v1.13 ΔG (E211A/S216R). More preferably, mutant v1.13 polypeptide of the invention has the amino acid sequence of SEQ ID NO: 18.

The mutant v1.13 polypeptide of the invention can, after administration to a host animal, preferably a mammal and more preferably a human, elicit antibodies, which can recognize wild-type meningococcal fHbp polypeptides of SEQ ID NO: 15. These antibodies are ideally bactericidal.

The inventors of WO2020/030782 also identified residues within the fHbp v1.15 sequence that can be modified to prevent binding to hfH. Such mutants are referred to herein as non-binding (NB) mutants. The inventors identified combinations of mutations in the v1.15 sequence that are particularly useful to prevent binding to hfH. fHbp v1.15 is also known in the art as fHbp variant B44.

The mature wild-type fHbp v1.15 lipoprotein from strain NM452 (GenBank Accession No. ABL14232.1) has the following amino acid sequence, with an N-terminal poly-glycine signal sequence being underlined:

(SEQ ID NO: 19)
CSSGGGGSGGGGVAADIGAGLADALTAPLDHKDKGLKSLTLEDSISQ
NGTLTLSAQGAERTFKAGDKDNSLNTGKLKNDKISRFDFIRQIEVDG
QLITLESGEFQVYKQSHSALTALQTEQVQDSEHSGKMVAKRQFRIGD
IVGEHTSFGKLPKDVMATYRGTAFGSDDAGGKLTYTIDFAAKQGHGK
IEHLKSPELNVDLAAADIKPDEKHHAVISGSVLYNQAEKGSYSLGIF
GGQAQEVAGSAEVETANGIRHIGLAAKQ

The mature v1.15 lipoprotein differs from the full-length wild-type sequence in that the full-length polypeptide has an additional 19 residue N-terminal leader sequence, which is cleaved from the mature polypeptide. Thus, full-length wild-type fHbp v1.15 has the following amino acid sequence (with the N-terminal leader sequence shown in bold font):

(SEQ ID NO: 46)
MNRTTFCCLSLTAALILTACSSGGGGSGGGGVAADIGAGLADALTAP
LDHKDKGLKSLTLEDSISQNGTLTLSAQGAERTFKAGDKDNSLNTGK
LKNDKISRFDFIRQIEVDGQLITLESGEFQVYKQSHSALTALQTEQV
QDSEHSGKMVAKRQFRIGDIVGEHTSFGKLPKDVMATYRGTAFGSDD
AGGKLTYTIDFAAKQGHGKIEHLKSPELNVDLAAADIKPDEKHHAVI
SGSVLYNQAEKGSYSLGIFGGQAQEVAGSAEVETANGIRHIGLAAKQ

The ΔG form of the mature v1.15 lipoprotein lacks the N-terminal poly-glycine sequence, i.e. it lacks the first 12 amino acids of SEQ ID NO: 19, and it lacks the first 31 amino acids of SEQ ID NO: 46:

(SEQ ID NO: 20)
VAADIGAGLADALTAPLDHKDKGLKSLTLEDSISQNGTLTLSAQGAE
RTFKAGDKDNSLNTGKLKNDKISRFDFIRQIEVDGQLITLESGEFQV
YKQSHSALTALQTEQVQDSEHSGKMVAKRQFRIGDIVGEHTSFGKLP
KDVMATYRGTAFGSDDAGGKLTYTIDFAAKQGHGKIEHLKSPELNVD
LAAADIKPDEKHHAVISGSVLYNQAEKGSYSLGIFGGQAQEVAGSAE
VETANGIRHIGLAAKQ

In one embodiment, the serogroup B antigenic component of the immunogenic composition of the invention comprises an amino acid sequence having at least k % sequence identity to SEQ ID NO: 20, with the proviso that the amino acid sequence of said mutant v1.15 meningococcal fHbp polypeptide includes a substitution mutation, i.e. a point mutation, at one or more of residues E214, S219 or E235 of SEQ ID NO: 20.

The value of k may be selected from 80, 85, 86, 87, 88, 89, 90, 91, 92, 93, 94, 95, 96, 97, 98, 99 or 100. k % is preferably 80% (i.e. the mutant fHbp v1.15 amino acid sequence has at least 80% identity to SEQ ID NO: 20) and is more preferably 85%, more preferably 90% and more preferably 95%. Most preferably, the mutant fHbp v1.15 amino acid sequence has at least 97%, at least 98% or at least 99% identity to SEQ ID NO: 20.

Preferably, the amino acid sequence differs from SEQ ID NO: 20 by at least one or more of the substitutions E214A, S219R or E235A. More preferably, the amino acid sequence comprises substitutions at residues selected from the following: (i) S219R, (ii) E214A and S219R, and (iii) E214A and E235A.

In preferred embodiments, a mutant v1.15 polypeptide has the amino acid sequence of SEQ ID NO: 21 (v.1.15_S219R), SEQ ID NO: 22 (v1.15_E214A/S219R) or SEQ ID NO: 23 (v1.15_E214A/E235A).

The mutant v1.15 polypeptide can, after administration to a host animal, preferably a mammal and more preferably a human, elicit antibodies which can recognize wild-type meningococcal fHbp polypeptides of SEQ ID NO: 19. These antibodies are ideally bactericidal.

The disclosure in WO2020/030782 also provides a fusion polypeptide comprising all three of v1, v2 and v3 meningococcal fHbp polypeptides, wherein the variant fHbp sequences are in the order v2-v3-v1 from N- to C-terminus. In a preferred embodiment, the serogroup B antigenic component of the immunogenic composition of the invention comprises such an fHbp fusion polypeptide.

Preferably, the fHbp fusion polypeptide has an amino acid sequence of formula NH₂-A-[—X-L]₃-B—COOH, wherein each X is a different variant fHbp sequence, L is an optional linker amino acid sequence, A is an optional N terminal amino acid sequence, and B is an optional C terminal amino acid sequence.

The v1 fHbp polypeptide component of the fusion is either a mutant v1.13 fHbp polypeptide or mutant v1.13 fHbp polypeptide as described above.

The v2 and v3 fHbp polypeptide components of the fusion for inclusion in the immunogenic composition against N. meningitidis serogroup B of the invention are preferably mutant v2 and v3 polypeptides having enhanced stability and reduced ability to bind to hfH, compared to the wild type v2 and v3 polypeptides.

As explained above, and without wishing to be bound to theory, reducing fHbp binding to hfH is advantageous because it prevents the formation of protective complexes between fHbp and hfH which can mask fHbp epitopes, and thereby increases the immunogenicity of the polypeptide antigens.

Residues within the v2 and v3 sequences, which can be modified to increase the stability of the polypeptide and also to reduce binding to hfH, have been identified and described in detail in WO2015/128480.

Full-length wild-type fHbp v2 from strain 2996 has the following amino acid sequence (leader sequence shown in bold font and poly-glycine sequence being underlined):

(SEQ ID NO: 24)
MNRTAFCCLSLTAALILTACSSGGGGVAADIGAGLADALTAPLDHKD
KSLQSLTLDQSVRKNEKLKLAAQGAEKTYGNGDSLNTGKLKNDKVSR
FDFIRQIEVDGQLITLESGEFQIYKQDHSAVVALQIEKINNPDKIDS
LINQRSFLVSGLGGEHTAFNQLPDGKAEYHGKAFSSDDAGGKLTYTI
DFAAKQGHGKIEHLKTPEQNVELAAAELKADEKSHAVILGDTRYGSE
EKGTYHLALFGDRAQEIAGSATVKIGEKVHEIGIAGKQ

The mature lipoprotein lacks the first 19 amino acids of SEQ ID NO: 24:

(SEQ ID NO: 25)
CSSGGGGVAADIGAGLADALTAPLDHKDKSLQSLTLDQSVRKNEKLK
LAAQGAEKTYGNGDSLNTGKLKNDKVSRFDFIRQIEVDGQLITLESG
EFQIYKQDHSAVVALQIEKINNPDKIDSLINQRSFLVSGLGGEHTAF
NQLPDGKAEYHGKAFSSDDAGGKLTYTIDFAAKQGHGKIEHLKTPEQ
NVELAAAELKADEKSHAVILGDTRYGSEEKGTYHLALFGDRAQEIAG
SATVKIGEKVHEIGIAGKQ

The ΔG form of SEQ ID NO: 24 lacks the first 26 amino acids:

(SEQ ID NO: 26)
VAADIGAGLADALTAPLDHKDKSLQSLTLDQSVRKNEKLKLAAQGAE
KTYGNGDSLNTGKLKNDKVSRFDFIRQIEVDGQLITLESGEFQIYKQ
DHSAVVALQIEKINNPDKIDSLINQRSFLVSGLGGEHTAFNQLPDGK
AEYHGKAFSSDDAGGKLTYTIDFAAKQGHGKIEHLKTPEQNVELAAA
ELKADEKSHAVILGDTRYGSEEKGTYHLALFGDRAQEIAGSATVKIG
EKVHEIGIAGKQ

In a preferred embodiment, the fusion polypeptide of the invention comprises a mutant v2 fHbp polypeptide comprising an amino acid sequence having at least k % sequence identity to SEQ ID NO: 26, with the proviso that the v2 fHbp amino acid sequence includes a substitution mutation, i.e. a point mutation, at residues S32 and L123 of SEQ ID NO: 26. Preferably the substitutions are S32V and L123R.

The value of k may be selected from 80, 85, 86, 87, 88, 89, 90, 91, 92, 93, 94, 95, 96, 97, 98, 99 or 100. k % is preferably 80% (i.e. the mutant fHbp v2 amino acid sequence has at least 80% identity to SEQ ID NO: 26) and is more preferably 85%, more preferably 90% and more preferably 95%.

In some embodiments, the fHbp v2 polypeptide included in the fusion protein of the invention is truncated relative to SEQ ID NO: 26. Compared to the wild-type mature sequence, SEQ ID NO: 26 is already truncated at the N-terminus up to and including the poly-glycine sequence (compare SEQ ID NOs: 25 and 26), but SEQ ID NO: 26 can be truncated at the C-terminus and/or further truncated at the N-terminus.

In a preferred embodiment, the v2 fHbp polypeptide included in the fusion protein of the invention comprises or consists of the amino acid sequence of SEQ ID NO: 30. The v2 fHbp polypeptide included in the fusion protein has, under the same experimental conditions, a higher stability than the same polypeptide but without the sequence differences at residues S32 and L123 e.g. higher stability than a wild-type meningococcal polypeptide consisting of SEQ ID NO: 24. The S32V mutation stabilizes the structure by introducing favorable hydrophobic interactions. Without wishing to be bound to theory, the L123R mutation abrogates fH binding by introducing clashes with fH and unfavorable charges.

The stability enhancement can be assessed using differential scanning calorimetry (DSC) e.g. as discussed in Johnson (2013) Arch Biochem Biophys 531:100-9 and Bruylants et al. Current Medicinal Chemistry 2005; 12:2011-20. DSC has previously been used to assess the stability of v2 fHbp (Johnson et al. PLoS Pathogen 2012; 8: e1002981). Suitable conditions for DSC to assess stability can use 20 pM of polypeptide in a buffered solution (e.g. 25 mM Tris) with a pH between 6 and 8 (e.g. 7-7.5) with 100-200 mM NaCl (e.g. 150 mM).

The increase in stability is evidenced by an at least 5° C., e.g. at least 10° C., 15° C., 20° C., 25° C., 30° C., 35° C. or more, increase in thermal transition midpoint (Tm) of at least one peak as compared to wildtype when assessed by DSC. Wild-type fHbp shows two DSC peaks during unfolding (one for the N-terminal domain and one for the C-terminal domain) and, where a v2 polypeptide included in the fusion protein of the invention includes both such domains, an “increase in stability” refers to an at least 5° C. increase in the Tₘ of the N-terminal domain. Tm of the N-terminal domain can occur at or even below 40° C. with wild-type v2 sequences (Johnson et al. (2012) PLoS Pathogen 8: e1002981), whereas C-terminal domains can have a Tm of 80° C. or more. Thus, the mutant fHbp v2 amino acid sequence included in the fusion protein of the invention preferably has a N-terminal domain with a Tm of at least 45° C. e.g. >50° C., >55° C., >60° C., >65° C., >70° C., >75° C., or even >80° C.

Full-length wild-type fHbp v3 from strain M1239 has the following amino acid sequence (leader sequence shown in bold font and poly-glycine sequence being underlined):

(SEQ ID NO: 27)
MNRTAFCCLSLTTALILTACSSGGGGSGGGGVAADIGTGLADALTAP
LDHKDKGLKSLTLEDSIPQNGTLTLSAQGAEKTFKAGDKDNSLNTGK
LKNDKISRFDFVQKIEVDGQTITLASGEFQIYKQNHSAVVALQIEKI
NNPDKTDSLINQRSFLVSGLGGEHTAFNQLPGGKAEYHGKAFSSDDP
NGRLHYSIDFTKKQGYGRIEHLKTLEQNVELAAAELKADEKSHAVIL
GDTRYGSEEKGTYHLALFGDRAQEIAGSATVKIGEKVHEIGIAGKQ

The mature lipoprotein lacks the first 19 amino acids of SEQ ID NO: 27:

(SEQ ID NO: 28)
CSSGGGGSGGGGVAADIGTGLADALTAPLDHKDKGLKSLTLEDSIPQ
NGTLTLSAQGAEKTFKAGDKDNSLNTGKLKNDKISRFDFVQKIEVDG
QTITLASGEFQIYKQNHSAVVALQIEKINNPDKTDSLINQRSFLVSG
LGGEHTAFNQLPGGKAEYHGKAFSSDDPNGRLHYSIDFTKKQGYGRI
EHLKTLEQNVELAAAELKADEKSHAVILGDTRYGSEEKGTYHLALFG
DRAQEIAGSATVKIGEKVHEIGIAGKQ

The ΔG form of SEQ ID NO: 27 lacks the first 31 amino acids (i.e. lacks the signal sequence and the poly-glycine sequence):

(SEQ ID NO: 29)
VAADIGTGLADALTAPLDHKDKGLKSLTLEDSIPQNGTLTLSAQGAE
KTFKAGDKDNSLNTGKLKNDKISRFDFVQKIEVDGQTITLASGEFQI
YKQNHSAVVALQIEKINNPDKTDSLINQRSFLVSGLGGEHTAFNQLP
GGKAEYHGKAFSSDDPNGRLHYSIDFTKKQGYGRIEHLKTLEQNVEL
AAAELKADEKSHAVILGDTRYGSEEKGTYHLALFGDRAQEIAGSATV
KIGEKVHEIGIAGKQ

In a preferred embodiment, the fusion polypeptide of the invention comprises a mutant v3 fHbp polypeptide comprising an amino acid sequence having at least k % sequence identity to SEQ ID NO: 29, with the proviso that the v3 fHbp amino acid sequence includes substitution mutations, i.e. point mutations, at residues S32 and L126 of SEQ ID NO: 29. Preferably the substitutions are S32V and L126R.

The value of k may be selected from 80, 85, 86, 87, 88, 89, 90, 91, 92, 93, 94, 95, 96, 97, 98, 99 or 100. k % is preferably 80% (i.e. the mutant fHbp v2 amino acid sequence has at least 80% identity to SEQ ID NO: 29) and is more preferably 85%, more preferably 90% and more preferably 95%.

In some embodiments, the fHbp v3 polypeptide included in the fusion protein of the invention is truncated relative to SEQ ID NO: 29. Compared to the wild-type mature sequence, SEQ ID NO: 29 is already truncated at the N-terminus up to and including the poly-glycine sequence (compare SEQ ID NOs: 28 and 29), but SEQ ID NO: 29 can be truncated at the C-terminus and/or further truncated at the N-terminus.

In a preferred embodiment, the v3 fHbp polypeptide included in the fusion protein of the invention comprises or consists of the amino acid sequence of SEQ ID NO: 31. The v3 fHbp polypeptide included in the fusion protein has, under the same experimental conditions, a higher stability than the same polypeptide but without the sequence differences at residues S32 and L126 e.g. higher stability than a wild-type meningococcal polypeptide consisting of SEQ ID NO: 27. The S32V mutation stabilizes the structure by introducing favorable hydrophobic interactions. Without wishing to be bound by theory, the L126R mutation abrogates fH binding by introducing clashes with fH and unfavorable charges.

The stability enhancement can be assessed using differential scanning calorimetry (DSC) e.g. as discussed in Johnson (2013) Arch Biochem Biophys 531:100-9 and Bruylants et al. (2005) Current Medicinal Chemistry 12:2011-20. DSC has previously been used to assess the stability of v3 fHbp (van der Veen et al. (2014) Infect Immun PMID 24379280). Suitable conditions for DSC to assess stability can use 20 pM of polypeptide in a buffered solution (e.g. 25 mM Tris) with a pH between 6 and 8 (e.g. 7-7.5) with 100-200 mM NaCl (e.g. 150 mM).

The increase in stability is evidenced by an at least 5° C., e.g. at least 10° C., 15° C., 20° C., 25° C., 30° C., 35° C. or more, increase in thermal transition midpoint (Tm) of at least one peak as compared to wildtype when assessed by DSC. Wild-type fHbp shows two DSC peaks during unfolding (one for the N-terminal domain and one for the C-terminal domain) and, where a v3 polypeptide included in the fusion protein of the invention includes both such domains, an “increase in stability” refers to an at least 5° C. increase in the Tₘ of the N-terminal domain. Tm of the N terminal domain can occur at around 60° C. or less with wild-type v3 sequences (Johnson et al. (2012) PLoS Pathogen 8:e1002981), whereas C-terminal domains can have a Tm of 80° C. or more. Thus, the mutant fHbp v3 amino acid sequence of the invention preferably has a N-terminal domain with a Tm of at least 65° C. e.g. >70° C., >75° C., or even >80° C.

As described above, in a preferred embodiment the fHbp fusion polypeptide has an amino acid sequence of formula NH₂-A-[—X-L]₃-B—COOH, wherein each X is a different variant fHbp sequence and L is an optional linker amino acid sequence. In a preferred embodiment, the linker amino acid sequence “L” is a glycine polymer or glycine-serine polymer linker.

Exemplary linkers include, but are not limited to, GGSG (SEQ ID NO:50), GGSGG (SEQ ID NO:51), GSGSG (SEQ ID NO:52), GSGGG (SEQ ID NO:53), GGGSG (SEQ ID NO:54), GSSSG (SEQ ID NO:55) and GSGGGG (SEQ ID NO:56). Other suitable glycine or glycine-serine polymer linkers will be apparent to the skilled person. In a preferred fusion polypeptide according to the invention, the v2 and v3 sequences and the v3 and v1 sequences are connected by the glycine-serine polymer linker GSGGGG (SEQ ID NO:56).

In a preferred embodiment, the fusion polypeptide of the invention comprises or consists of one of the following amino acid sequences (glycine-serine linker sequences are underlined and mutated residues are indicated in bold font):

fHbp 23S_1.13_E211A/E232A
(SEQ ID NO: 32)
VAADIGAGLADALTAPLDHKDKSLQSLTLDQVVRKNEKLKLAAQGAE
KTYGNGDSLNTGKLKNDKVSRFDFIRQIEVDGQLITLESGEFQIYKQ
DHSAVVALQIEKINNPDKIDSLINQRSFRVSGLGGEHTAFNQLPDGK
AEYHGKAFSSDDAGGKLTYTIDFAAKQGHGKIEHLKTPEQNVELAAA
ELKADEKSHAVILGDTRYGSEEKGTYHLALFGDRAQEIAGSATVKIG
EKVHEIGIAGKQGSGGGGVAADIGTGLADALTAPLDHKDKGLKSLTL
EDVIPQNGTLTLSAQGAEKTFKAGDKDNSLNTGKLKNDKISRFDFVQ
KIEVDGQTITLASGEFQIYKQNHSAVVALQIEKINNPDKTDSLINQR
SFRVSGLGGEHTAFNQLPGGKAEYHGKAFSSDDPNGRLHYSIDFTKK
QGYGRIEHLKTLEQNVELAAAELKADEKSHAVILGDTRYGSEEKGTY
HLALFGDRAQEIAGSATVKIGEKVHEIGIAGKQGSGGGGVAADIGAG
LADALTAPLDHKDKGLQSLTLDQSVRKNEKLKLAAQGAEKTYGNGDS
LNTGKLKNDKVSRFDFIRQIEVDGKLITLESGEFQVYKQSHSALTAL
QTEQVQDSEDSGKMVAKRQFRIGDIAGEHTSFDKLPKGGSATYRGTA
FGSDDAGGKLTYTIDFAAKQGHGKIEHLKSPELNVELATAYIKPDEK
RHAVISGSVLYNQDAKGSYSLGIFGGQAQEVAGSAAVETANGIHHIG
LAAKQ
fHbp 23S_1.13_E211A/S216R
(SEQ ID NO: 33)
VAADIGAGLADALTAPLDHKDKSLQSLTLDQVVRKNEKLKLAAQGAE
KTYGNGDSLNTGKLKNDKVSRFDFIRQIEVDGQLITLESGEFQIYKQ
DHSAVVALQIEKINNPDKIDSLINQRSFRVSGLGGEHTAFNQLPDGK
AEYHGKAFSSDDAGGKLTYTIDFAAKQGHGKIEHLKTPEQNVELAAA
ELKADEKSHAVILGDTRYGSEEKGTYHLALFGDRAQEIAGSATVKIG
EKVHEIGIAGKQGSGGGGVAADIGTGLADALTAPLDHKDKGLKSLTL
EDVIPQNGTLTLSAQGAEKTFKAGDKDNSLNTGKLKNDKISRFDFVQ
KIEVDGQTITLASGEFQIYKQNHSAVVALQIEKINNPDKTDSLINQR
SFRVSGLGGEHTAFNQLPGGKAEYHGKAFSSDDPNGRLHYSIDFTKK
QGYGRIEHLKTLEQNVELAAAELKADEKSHAVILGDTRYGSEEKGTY
HLALFGDRAQEIAGSATVKIGEKVHEIGIAGKQGSGGGGVAADIGAG
LADALTAPLDHKDKGLQSLTLDQSVRKNEKLKLAAQGAEKTYGNGDS
LNTGKLKNDKVSRFDFIRQIEVDGKLITLESGEFQVYKQSHSALTAL
QTEQVQDSEDSGKMVAKRQFRIGDIAGEHTSFDKLPKGGSATYRGTA
FGSDDAGGKLTYTIDFAAKQGHGKIEHLKSPELNVELATAYIKPDEK
RHAVISGSVLYNQDAKGSYRLGIFGGQAQEVAGSAEVETANGIHHIG
LAAKQ
fHbp_23S_1.15_S219R
(SEQ ID NO: 34)
VAADIGAGLADALTAPLDHKDKSLQSLTLDQVVRKNEKLKLAAQGAE
KTYGNGDSLNTGKLKNDKVSRFDFIRQIEVDGQLITLESGEFQIYKQ
DHSAVVALQIEKINNPDKIDSLINQRSFRVSGLGGEHTAFNQLPDGK
AEYHGKAFSSDDAGGKLTYTIDFAAKQGHGKIEHLKTPEQNVELAAA
ELKADEKSHAVILGDTRYGSEEKGTYHLALFGDRAQEIAGSATVKIG
EKVHEIGIAGKQGSGGGGVAADIGTGLADALTAPLDHKDKGLKSLTL
EDVIPQNGTLTLSAQGAEKTFKAGDKDNSLNTGKLKNDKISRFDFVQ
KIEVDGQTITLASGEFQIYKQNHSAVVALQIEKINNPDKTDSLINQR
SFRVSGLGGEHTAFNQLPGGKAEYHGKAFSSDDPNGRLHYSIDFTKK
QGYGRIEHLKTLEQNVELAAAELKADEKSHAVILGDTRYGSEEKGTY
HLALFGDRAQEIAGSATVKIGEKVHEIGIAGKQGSGGGGVAADIGAG
LADALTAPLDHKDKGLKSLTLEDSISQNGTLTLSAQGAERTFKAGDK
DNSLNTGKLKNDKISRFDFIRQIEVDGQLITLESGEFQVYKQSHSAL
TALQTEQVQDSEHSGKMVAKRQFRIGDIVGEHTSFGKLPKDVMATYR
GTAFGSDDAGGKLTYTIDFAAKQGHGKIEHLKSPELNVDLAAADIKP
DEKHHAVISGSVLYNQAEKGSYRLGIFGGQAQEVAGSAEVETANGIR
HIGLAAKQ
fHbp_23S_1.15_E214A/S219R
(SEQ ID NO: 35)
VAADIGAGLADALTAPLDHKDKSLQSLTLDQVVRKNEKLKLAAQGAE
KTYGNGDSLNTGKLKNDKVSRFDFIRQIEVDGQLITLESGEFQIYKQ
DHSAVVALQIEKINNPDKIDSLINQRSFRVSGLGGEHTAFNQLPDGK
AEYHGKAFSSDDAGGKLTYTIDFAAKQGHGKIEHLKTPEQNVELAAA
ELKADEKSHAVILGDTRYGSEEKGTYHLALFGDRAQEIAGSATVKIG
EKVHEIGIAGKQGSGGGGVAADIGTGLADALTAPLDHKDKGLKSLTL
EDVIPQNGTLTLSAQGAEKTFKAGDKDNSLNTGKLKNDKISRFDFVQ
KIEVDGQTITLASGEFQIYKQNHSAVVALQIEKINNPDKTDSLINQR
SFRVSGLGGEHTAFNQLPGGKAEYHGKAFSSDDPNGRLHYSIDFTKK
QGYGRIEHLKTLEQNVELAAAELKADEKSHAVILGDTRYGSEEKGTY
HLALFGDRAQEIAGSATVKIGEKVHEIGIAGKQGSGGGGVAADIGAG
LADALTAPLDHKDKGLKSLTLEDSISQNGTLTLSAQGAERTFKAGDK
DNSLNTGKLKNDKISRFDFIRQIEVDGQLITLESGEFQVYKQSHSAL
TALQTEQVQDSEHSGKMVAKRQFRIGDIVGEHTSFGKLPKDVMATYR
GTAFGSDDAGGKLTYTIDFAAKQGHGKIEHLKSPELNVDLAAADIKP
DEKHHAVISGSVLYNQAAKGSYRLGIFGGQAQEVAGSAEVETANGIR
HIGLAAKQ
fHbp_23S_1.15_E214A/E235A
(SEQ ID NO: 36)
VAADIGAGLADALTAPLDHKDKSLQSLTLDQVVRKNEKLKLAAQGAE
KTYGNGDSLNTGKLKNDKVSRFDFIRQIEVDGQLITLESGEFQIYKQ
DHSAVVALQIEKINNPDKIDSLINQRSFRVSGLGGEHTAFNQLPDGK
AEYHGKAFSSDDAGGKLTYTIDFAAKQGHGKIEHLKTPEQNVELAAA
ELKADEKSHAVILGDTRYGSEEKGTYHLALFGDRAQEIAGSATVKIG
EKVHEIGIAGKQGSGGGGVAADIGTGLADALTAPLDHKDKGLKSLTL
EDVIPQNGTLTLSAQGAEKTFKAGDKDNSLNTGKLKNDKISRFDFVQ
KIEVDGQTITLASGEFQIYKQNHSAVVALQIEKINNPDKTDSLINQR
SFRVSGLGGEHTAFNQLPGGKAEYHGKAFSSDDPNGRLHYSIDFTKK
QGYGRIEHLKTLEQNVELAAAELKADEKSHAVILGDTRYGSEEKGTY
HLALFGDRAQEIAGSATVKIGEKVHEIGIAGKQGSGGGGVAADIGAG
LADALTAPLDHKDKGLKSLTLEDSISQNGTLTLSAQGAERTFKAGDK
DNSLNTGKLKNDKISRFDFIRQIEVDGQLITLESGEFQVYKQSHSAL
TALQTEQVQDSEHSGKMVAKRQFRIGDIVGEHTSFGKLPKDVMATYR
GTAFGSDDAGGKLTYTIDFAAKQGHGKIEHLKSPELNVDLAAADIKP
DEKHHAVISGSVLYNQAAKGSYSLGIFGGQAQEVAGSAAVETANGIR
HIGLAAKQ

In a preferred embodiment, the fusion polypeptide of the invention comprises the amino acid sequence of SEQ ID NO. 33. In an alternative preferred embodiment, the fusion polypeptide of the invention comprises the amino acid sequence of SEQ ID NO. 32.

The fusion polypeptide of the invention can, after administration to a host animal, preferably a mammal and more preferably a human, elicit antibodies which can recognise wild-type meningococcal fHbp polypeptides, in particular the polypeptides of SEQ ID NO: 45, 46, 24 and/or 27. These antibodies are ideally bactericidal.

As described above, in a preferred embodiment an fHbp fusion polypeptide according to the invention has an amino acid sequence of formula NH₂-A-[—X-L]₃-B—COOH, wherein each X is a different variant fHbp sequence and A is an optional N terminal amino acid sequence. In preferred embodiments, fusion proteins described herein further comprise the following N-terminal amino acid sequence, which is advantageous for enabling good expression of the fusion protein:

(SEQ ID NO. 48)
MGPDSDRLQQRR

Any of the fusion proteins disclosed herein (e.g. SEQ ID Nos. 32-36, 43 and 44) may be modified to include the amino acid sequence of SEQ ID NO. 48 at the N-terminal of the fusion polypeptide, i.e. the amino acid sequence of SEQ ID NO. 48 is added to the N-terminal of the fHbp v2 component of the fusion polypeptide.

In a preferred embodiment, the serogroup B antigenic component of the immunogenic composition of the invention comprises the complete BEXSERO vaccine product, together with an fHbp fusion polypeptide as defined above. Most preferably, the fHbp fusion polypeptide is fHbp 23S_1.13_E211A/S216R. Preferably, the serogroup B antigenic component is provided in a single fully liquid formulation.

Preferred v1.13, v1.15 and/or fusion polypeptides described above can elicit antibody responses that are bactericidal against meningococci. Bactericidal antibody responses are conveniently measured in mice and are a standard indicator of vaccine efficacy (e.g. see end-note 14 of Pizza et al. (2000) Science 287:1816-1820; also WO2007/028408).

Polypeptides described above can preferably elicit an antibody response which is bactericidal against a N. meningitidis serogroup B strain which expresses a v1.13 fHbp sequence.

Preferred polypeptides described above can elicit antibodies in a mouse which are bactericidal against a N. meningitidis strain which expresses a v1.13 fHbp sequence in a serum bactericidal assay.

Polypeptides described above can preferably elicit an antibody response which is bactericidal against a N. meningitidis serogroup B strain which expresses a v1.15 fHbp sequence.

Preferred polypeptides described above can elicit antibodies in a mouse which are bactericidal against a N. meningitidis strain which expresses a v1.15 fHbp sequence in a serum bactericidal assay.

For example, an immunogenic composition comprising these polypeptides can provide a serum bactericidal titer of >1:4 using the Goldschneider assay with human complement [Goldschneider et al. (1969) J. Exp. Med. 129:1307-26, Santos et al. (2001) Clinical and Diagnostic Laboratory Immunology 8:616-23, and Frasch et al. (2009) Vaccine 27S:B112-6], and/or providing a serum bactericidal titer of >1:128 using baby rabbit complement.

Polypeptides described above can be prepared by various means e.g. by chemical synthesis (at least in part), by digesting longer polypeptides using proteases, by translation from RNA, by purification from cell culture (e.g. from recombinant expression or from N. meningitidis culture), etc.

Heterologous expression in an E. coli host is a preferred expression route.

Polypeptides are ideally at least 100 amino acids long e.g. 150aa, 175aa, 200aa, 225aa, or longer. They include a mutant fHbp v1, v2 and/or v3 amino acid sequence, and the mutant fHbp v1, v2 or v3 amino acid sequence should similarly be at least 100 amino acids long e.g. 150aa, 175aa, 200aa, 225aa, or longer.

The fHbp is naturally a lipoprotein in N. meningitidis. It has also been found to be lipidated when expressed in E. coli with its native leader sequence or with heterologous leader sequences. Polypeptides of the invention may have an N-terminus cysteine residue, which may be lipidated e.g. comprising a palmitoyl group, usually forming tripalmitoyl-S-glyceryl-cysteine. In other embodiments the polypeptides are not lipidated.

Polypeptides are preferably prepared in substantially pure or substantially isolated form (i.e. substantially free from other Neisserial or host cell polypeptides). In general, the polypeptides are provided in a non-naturally occurring environment e.g. they are separated from their naturally-occurring environment. In certain embodiments, the polypeptide is present in a composition that is enriched for the polypeptide as compared to a starting material. Thus, purified polypeptide is provided, whereby purified means that the polypeptide is present in a composition that is substantially free of other expressed polypeptides, whereby substantially free is meant that more than 50% (e.g. >75%, >80%, >90%, >95%, or >99%) of total polypeptide in the composition is a polypeptide of the invention.

Polypeptides can take various forms (e.g. native, fusions, glycosylated, non-glycosylated, lipidated, disulfide bridges, etc.).

If a polypeptide is produced by translation in a biological host then a start codon is required, which will provide a N-terminus methionine in most hosts. Thus, a polypeptide will, at least at a nascent stage, include a methionine residue upstream of said SEQ ID NO sequence.

Cleavage of nascent sequences means that the mutant fHbp v1, v2 or v3 amino acid sequence might itself provide the polypeptide N-terminus. In other embodiments, however, a polypeptide can include a N-terminal sequence upstream of the mutant fHbp v1, v2 or v3 amino acid sequence. In some embodiments the polypeptide has a single methionine at the N-terminus immediately followed by the mutant fHbp v1, v2 or v3 amino acid sequence; in other embodiments a longer upstream sequence may be used. Such an upstream sequence may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. a histidine tag i.e. Hisₙ where n is 4, 5, 6, 7, 8, 9, 10 or more). Other suitable N-terminal amino acid sequences will be apparent to those skilled in the art.

A polypeptide may also include amino acids downstream of the final amino acid of the mutant fHbp v1, v2 or v3 amino acid sequence. Such C-terminal extensions may be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising a histidine tag i.e. Hisₙ where n=4, 5, 6, 7, 8, 9, 10 or more), or sequences which enhance polypeptide stability. Other suitable C-terminal amino acid sequences will be apparent to those skilled in the art.

In some embodiments, the invention excludes polypeptides which include a histidine tag (cf. Johnson et al. (2012) PLoS Pathogen 8:e1002981, and Pajon et al. (2012) Infect Immun 80:2667-77), and in particular a hexahistidine tag at the C-terminus.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains.

Polypeptides may be attached or immobilized to a solid support.

Polypeptides may comprise a detectable label e.g. a radioactive label, a fluorescent label, or a biotin label. This is particularly useful in immunoassay techniques.

Polypeptides typically consist of an artificial amino acid sequence, namely a sequence which is not present in any naturally-occurring meningococci.

Affinity for factor H can be quantitatively assessed using surface plasmon resonance (e.g. as disclosed in Schneider et al. (2009) Nature 458:890-5) with immobilized human fH. Mutations which provide an affinity reduction (i.e. an increase in the dissociation constant, KD) of at least 10-fold, and ideally at least 100-fold, is preferred (when measured under the same experimental conditions relative to the same polypeptide but without the mutation).

### Meningococcus Serogroups A, C, W135 and Y

The present immunogenic composition against N. meningitidis serogroup B in liquid form described above can be used for the reconstitution of an immunogenic composition against N. meningitidis serogroups A, C, W135, and Y, in a solid form (e.g. freeze-dried), comprising capsular saccharides of the meningococcal serogroups A, C, W135, and Y that are conjugated to carrier protein(s). The capsular saccharides of each of these four serogroups ACWY of N. meningitidis are well characterized.

The capsular saccharide of serogroup A meningococcus is a homopolymer of (α1→6)-linked N-acetyl-D-mannosamine-1-phosphate, with partial 0-acetylation in the C3 and C4 positions. The acetyl groups can be replaced with blocking groups to prevent hydrolysis (see, e.g., WO03/080678, which is incorporated herewith by reference), and such modified saccharides are still serogroup A capsular saccharides as disclosed herein.

The serogroup C capsular saccharide is a homopolymer of (α2→9)-linked sialic acid (N-acetyl neuraminic acid (“NeuNAc”)). Most serogroup C strains have 0-acetyl groups at C-7 and/or C-8 of the sialic acid residues, but about 15% of clinical isolates lack these 0-acetyl groups. The saccharide structure is written as →9)-NeupoNAc 7/8OAc-(α2→.

The serogroup W135 saccharide is a polymer of sialic acid-galactose disaccharide units. Like the serogroup C saccharide, it has variable 0-acetylation, but at sialic acid 7 and 9 positions. The structure is written as: →4)-D-Neu p5Ac(7/9OAc)-α-(2→6)-D-Gal-α-(1→.

The serogroup Y saccharide is similar to the serogroup W135 saccharide, except that the disaccharide repeating unit includes glucose instead of galactose. Like serogroup W135, it has variable O-acetylation at sialic acid 7 and 9 positions. The serogroup Y structure is written as: →4)-D-Neup5Ac(7/9OAc)-α-(2→6)-D-Glc-α-(1→.

The capsular saccharides may be native capsular saccharides obtained from a meningococcal bacterium of the related serogroup. The native capsular saccharide may be modified by any method available to one with ordinary skills in the art so long as the capsular saccharide retains at least one epitope that elicits serum bactericidal antibodies. Exemplary modifications are detailed below. In addition to native and modified capsular saccharides, the capsular saccharides may be chemically synthesized as long as the synthesized compound (saccharide, saccharide analog, etc.) includes at least one epitope that elicits serum bactericidal antibodies that bind to capsular saccharides. All such native, modified and chemically synthesized capsular saccharides are within the scope of the meningococcal capsular saccharides disclosed herein. Exemplary modifications and chemical syntheses are described below.

The capsular saccharides in the vaccines may be O-acetylated as described above (e.g., with the same O-acetylation pattern as seen in native capsular saccharides), or they may be partially or totally de-O-acetylated at one or more positions of the saccharide rings, or they may be hyper-O-acetylated relative to the native capsular saccharides.

The capsular saccharides in the vaccines may be shorter than the native capsular saccharides seen in bacteria. Thus, the saccharides may be partially depolymerized, which typically occurs after purification but before conjugation. Depolymerization reduces the chain length of the saccharides up to the formation of saccharides of the desired size. A depolymerization method involves the use of hydrogen peroxide which may be added to a saccharide (e.g., to give a final H₂O₂ concentration of 1%), and the mixture is then incubated (e.g., at about 55° C.) until a desired chain length reduction has been achieved. Another depolymerization method involves acid hydrolysis (see, e.g., WO03/007985, which is incorporated herewith by reference). Other depolymerization methods are known to any skilled person. The capsular saccharides used in the vaccines may be obtainable by any of these depolymerization methods. Depolymerization can be used in order to provide an optimum chain length for immunogenicity and/or to reduce chain length for physical manageability of the saccharides. Native capsular saccharides are typically referred to as capsular polysaccharides while depolymerized capsular saccharides are typically referred to as capsular oligosaccharides.

In this composition capsular saccharides may be used in the form of oligosaccharides. These are conveniently formed by fragmentation of purified capsular polysaccharide (e.g. by hydrolysis), which will usually be followed by purification of the fragments of the desired size.

The vaccine products marketed under the trade names MENVEO, MENACTRA, and NIMENRIX all contain conjugated capsular saccharide antigens from each of serogroups Y, W135, C and A.

In MENVEO (also known generically as Meningococcal (Groups A, C, Y, and W-135) Oligosaccharide Diphtheria CRM197 Conjugate Vaccine) each of the Men A, C, W135 and Y antigens is conjugated to a CRM197 carrier.

In MENACTRA (also known generically as Meningococcal (Groups A, C, Y and W-135) Polysaccharide Diphtheria Toxoid Conjugate Vaccine) each of the A, C, W135 and Y antigens is conjugated to a diptheria toxoid carrier.

In MENQUADFI (also known generically as Meningococcal (Groups A, C, Y and W-135) Polysaccharide Conjugate Vaccine) each of the A, C, W135 and Y antigens is conjugated to a tetanus toxoid carrier.

In NIMENRIX (also known generically as Meningococcal polysaccharide groups A, C, W135 and Y conjugate vaccine) each of the Men A, C, W135 and Y antigens is conjugated to a tetanus toxoid carrier.

In preferred embodiments, the immunogenic composition in solid form, to be reconstituted with the present Men B liquid composition, comprises the Men A, C, W135 and Y antigen conjugates which are present in MENVEO, the Men A, C, W135 and Y antigen conjugates, which are present in MENACTRA, the Men A, C, W135 and Y antigen conjugates, which are present in MENQUADFI, or the Men A, C, W135 and Y antigen conjugates which are present in NIMENRIX. In an embodiment, the immunogenic composition in solid form of this invention comprises the Men A, C, W135 and Y antigen conjugates, wherein the capsular saccharides and/or oligosaccharides thereof are conjugated to CRM197 carrier, including recombinant CRM197 carrier (rCRM₁₉₇), a tetanus toxoid carrier (TT), or a diptheria toxoid carrier (DT). In an embodiment, the immunogenic composition in solid form of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇), or a tetanus toxoid carrier (TT).

Preferably, the immunogenic composition in solid form of this invention comprises the Men A, C, W135 and Y antigen conjugates, wherein the capsular saccharides and/or oligosaccharides thereof are conjugated to CRM₁₉₇ carrier, including recombinant CRM₁₉₇ carrier (rCRM₁₉₇).

### Further Antigenic Components

Immunogenic compositions of this invention may include antigens for immunizing against other diseases or infections. For example, the composition may include one or more of the following further antigens:
a saccharide antigen from Streptococcus pneumoniae [e.g. Watson (2000) Pediatr Infect Dis J 19:331-332, Rubin (2000) Pediatr Clin North Am 47:269-285, and Jedrzejas (2001) Microbiol Mol Biol Rev 65:187-207].
an antigen from hepatitis A virus, such as inactivated virus [e.g. Bell (2000) Pediatr Infect Dis J 19:1187-1188, Iwarson (1995) APMIS 103:321-326].
an antigen from hepatitis B virus, such as the surface and/or core antigens [e.g. Gerlich et al. (1990) Vaccine 8 Suppl: S63-68 & 79-80].
a diphtheria antigen, such as a diphtheria toxoid [e.g. chapter 3 of Vaccines (1988) eds. Plotkin & Mortimer. ISBN 0-7216-1946-0]e.g. the CRM197 mutant [e.g. Del Guidice et al (1998) Molecular Aspects of Medicine 19:1-70].
a tetanus antigen, such as a tetanus toxoid (e.g. chapter 4 of Vaccines (1988) eds. Plotkin & Mortimer. ISBN 0-7216-1946-0).
an antigen from Bordetella pertussis, such as pertussis holotoxin (PT) and filamentous haemagglutinin (FHA) from B. pertussis, optionally also in combination with pertactin and/or agglutinogens 2 and 3 (e.g. Gustafsson et al. (1996) N. Engl. J. Med. 334:349-355, and Rappuoli et al. (1991) TIBTECH 9:232-238).
a saccharide antigen from Haemophilus influenzae B [e.g. Costantino et al (1999) Vaccine 17:1251-1263].
polio antigen(s) [e.g. Sutter et al (2000) Pediatr Clin North Am 47:287-308, and Zimmerman & Spann (1999) Am Fam Physician 59:113-118, 125-126], such as IPV.
measles, mumps and/or rubella antigens (e.g. chapters 9, 10 & 11 of Vaccines (1988) eds. Plotkin & Mortimer. ISBN 0-7216-1946-0).
influenza antigen(s) (e.g. chapter 19 of Vaccines (1988) eds. Plotkin & Mortimer. ISBN 0-7216-1946-0), such as the haemagglutinin and/or neuraminidase surface proteins.
an antigen from Moraxella catarrhalis [e.g. McMichael (2000) Vaccine 19 Suppl 1: S101-107].
a protein antigen from Streptococcus agalactiae (group B streptococcus) [e.g. Schuchat (1999) Lancet 353(9146):51-6, WO02/34771].
a saccharide antigen from Streptococcus agalactiae (group B streptococcus).
an antigen from Streptococcus pyogenes (group A streptococcus) [e.g. WO02/34771, Dale (1999) Infect Dis Clin North Am 13:227-43, Ferretti et al (2001) PNAS USA 98: 4658-4663].
an antigen from Staphylococcus aureus [e.g. Kuroda et al (2001) Lancet 357(9264):1225-1240; see also pages 1218-1219].

In an embodiment the immunogenic compositions of the present invention do not comprise an antigen against N. meningitidis serogroup X.

Toxic protein antigens may be detoxified where necessary (e.g. detoxification of pertussis toxin by chemical and/or genetic means [Rappuoli et al (1991) TIBTECH 9:232-238]).

Where a diphtheria antigen is included in the composition it is preferred also to include tetanus antigen and pertussis antigens. Similarly, where a tetanus antigen is included, it is preferred also to include diphtheria and pertussis antigens. Similarly, where a pertussis antigen is included, it is preferred also to include diphtheria and tetanus antigens. DTP combinations are thus preferred.

Saccharide antigens are preferably in the form of conjugates. In general, conjugation enhances the immunogenicity of saccharides as it converts them from T-independent antigens to T-dependent antigens, thus allowing priming for immunological memory. Conjugation is particularly useful for paediatric vaccines and is a well-known technique.

Typical carrier proteins are bacterial toxins, such as diphtheria or tetanus toxins, or toxoids or mutants thereof. The CRM197 diphtheria toxin mutant [Research Disclosure, 453077 (January 2002)] is useful, and is the carrier in the Streptococcus pneumoniae vaccine sold under the trade name PREVNAR. Also useful as carrier protein is recombinant CRM₁₉₇ (rCRM₁₉₇) obtained or derived from Pseudomonas fluorescens or from Escherichia coli. Other suitable carrier proteins include the N. meningitidis outer membrane protein complex [EP-A-0372501], synthetic peptides [EP-A-0378881, EP-A-0427347], heat shock proteins [WO93/17712, WO94/03208], pertussis proteins [WO98/58668, EP-A-0471177], cytokines [WO91/01146], lymphokines [WO91/01146], hormones [WO91/01146], growth factors [WO91/01146], artificial proteins comprising multiple human CD4⁺ T cell epitopes from various pathogen-derived antigens [Falugi et al. (2001) Eur J Immunol 31:3816-3824] such as N19 [Baraldo et al. (2004) Infect Immun 72(8):4884-7], protein D from H. influenzae [EP-A-0594610, Ruan et al. (1990) J Immunol 145:3379-3384] pneumolysin [Kuo et al. (1995) Infect Immun 63:2706-13] or its non-toxic derivatives [Michon et al. (1998) Vaccine. 16:1732-41], pneumococcal surface protein PspA [WO02/091998], iron-uptake proteins [WO01/72337], toxin A or B from C. difficile [WO0/61761], recombinant P. aeruginosa exoprotein A (rEPA) [WO0/33882], etc.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [Lees et al. (1996) Vaccine 14:190-198, WO95/08348]). Other suitable techniques use carbodiimides, hydrazides, active esters, norbornane, p-nitrobenzoic acid, N-hydroxysuccinimide, S-NHS, EDC, TSTU, etc.

Linkages via a linker group may be made using any known procedure, for example, the procedures described in U.S. Pat. Nos. 4,882,317 and 4,695,624. One type of linkage involves reductive amination of the polysaccharide, coupling the resulting amino group with one end of an adipic acid linker group, and then coupling a protein to the other end of the adipic acid linker group [Porro et al. (1985) Mol Immunol 22:907-919, EP0208375]. Other linkers include B-propionamido [WO0/10599], nitrophenyl-ethylamine [Gever et al. Med. Microbiol. Immunol, 165: 171-288 (1979)], haloacyl halides [U.S. Pat. No. 4,057,685], glycosidic linkages [U.S. Pat. Nos. 4,673,574; 4,761,283; 4,808,700], 6-aminocaproic acid [U.S. Pat. No. 4,459,286], ADH [U.S. Pat. No. 4,965,338], C₄ to C₁₂ moieties [U.S. Pat. No. 4,663,160] etc. As an alternative to using a linker, direct linkage can be used. Direct linkages to the protein may comprise oxidation of the polysaccharide followed by reductive amination with the protein, as described in, for example, U.S. Pat. Nos. 4,761,283 and 4,356,170.

A process involving the introduction of amino groups into the saccharide (e.g. by replacing terminal ═O groups with —NH₂) followed by derivatisation with an adipic diester (e.g. adipic acid N-hydroxysuccinimido diester) and reaction with carrier protein is preferred. Another preferred reaction uses CDAP activation with a protein D carrier e.g. for MenA or MenC.

Antigens in the composition will typically be present at a concentration of at least 1 μg/ml each. In general, the concentration of any given antigen will be sufficient to elicit an immune response against that antigen.

Immunogenic compositions of the invention may be used therapeutically (i.e. to treat an existing infection) or prophylactically (i.e. to prevent future infection).

As an alternative to using protein antigens in the immunogenic compositions of the invention, nucleic acid (which could be RNA, such as a self-replicating RNA, or DNA, such as a plasmid) encoding the antigen may be used.

### Non-Antigenic Components and Formulation of the Compositions

The immunogenic composition of the invention will generally include a pharmaceutically acceptable excipient, which can be any substance that does not itself induce the production of antibodies harmful to the patient receiving the composition, and which can be administered without undue toxicity. Depending on the formulation of the immunogenic compositions described below in more details, pharmaceutically acceptable excipients can include liquids such as water, saline, glycerol and ethanol. Auxiliary substances, such as wetting or emulsifying agents, pH buffering substances, and the like, can also be present in such formulations. A thorough discussion of suitable excipients is available in Gennaro (2000) Remington: The Science and Practice of Pharmacy. 20th edition, ISBN: 0683306472.

The immunogenic compositions are preferably sterile.

The immunogenic composition against N. meningitidis serogroup B of this invention is formulated as a liquid “adsorbed” vaccine, with the protein antigens adsorbed onto an adsorbing agent, e.g. a compound containing aluminum, and comprises one or more pharmaceutically acceptable tonicity modifying agents.

A “pharmaceutically acceptable tonicity modifying agent” is a compound that is physiologically tolerated and imparts a suitable tonicity to a formulation to prevent the net flow of water across cell membranes that are in contact with the formulation. In some embodiments, the tonicity modifying agent used for the composition is a salt (or mixtures of salts), preferably selected from sodium chloride, sugars, such as sucrose and sorbitol, and mixtures thereof. More preferably, the tonicity modifying agents in these compositions are mixtures of sodium chloride and sucrose; most preferably, they are aqueous solutions of sodium chloride and sucrose.

In a preferred embodiment, the immunogenic composition against N. meningitidis serogroup B of this invention is a liquid formulation, e.g. an aqueous solution, having a concentration of sodium chloride up to about 3.8 mg/ml, preferably lower than about 3.0 mg/ml, more preferably of about 2.8 mg/ml. Values intermediate to the above said concentrations of sodium chloride are also intended to be part of this invention (e.g. 3.7, 3.6, 3.5, 3.4, 3.3, 3.2, 3.1, and 2.9 mg/ml), as well as values lower than 2.8 mg/ml. In further aspects of this invention, the concentration of sodium chloride in the Men B liquid formulation may ranges between 1.0 mg/ml and 3.8 mg/ml, between 1.5 mg/ml and 3.5 mg/ml, between 2.0 and 3.5 mg/ml, between 2.5 and 3.5 mg/ml, or between 2.5 mg/ml and 3.0 mg/ml.

In a further preferred embodiment, the immunogenic composition against N. meningitidis serogroup B of this invention comprises an aqueous solution having a concentration of sucrose in the range of about 2-3% by weight with respect to the total volume of the solution (referred to in the following as “w/v”), preferably of about 3% w/v. In further aspects of this invention, the concentration of sucrose in the immunogenic composition of Men B antigens may range from 2.0 mg/ml and 3.5 mg/ml, between 2.5 mg/ml and 3.5 mg/ml, or between 2.5 mg/ml and 3.0 mg/ml.

In an aspect of this invention, the immunogenic composition against N. meningitidis serogroup B of this invention may be adjuvanted. Adjuvants which may be used in this composition include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolines, or mixtures thereof. Other substances that act as immunostimulating agents are disclosed in chapter 7 of Vaccine Design . . . (1995) eds. Powell & Newman. ISBN: 030644867X. Plenum.

The use of a compound containing aluminum as the adsorbing agent is preferred, and all MenB antigens in the present immunogenic composition, including fusion fHbp polypeptides are generally adsorbed to this compound. Particularly preferred as adsorbing agent in the present liquid composition is a compound selected from aluminum salts and mixtures thereof, including aluminum hydroxide (Alum), aluminum phosphate, potassium aluminum sulphate, oxyhydroxides and hydroxyphosphates (e.g. see chapters 8 & 9 of Vaccine Design . . . (1995) eds. Powell & Newman. ISBN: 030644867X. Plenum). The salts can take any suitable form (e.g. gel, crystalline, amorphous, etc.). In a most preferred embodiment, this composition in liquid formulation is adjuvanted with Alum as adsorbing agent for the protein antigens. In an aspect, the absorbing agent, preferably Alum, is present at a concentration ranging from 2 to 5%, e.g. 3%, by weight with respect to the total volume of the present Men B antigens immunogenic composition in liquid form. Further concentrations of the absorbing agent for instance in the ranges from 2 to 4% or from 2.5 to 3.5% are intended to be part of this invention too.

This immunogenic composition against N. meningitidis serogroup B in liquid formulation is preferably buffered e.g. at between pH 6 and pH 8, preferably around pH 6.5. An appropriate buffer may be selected from acetate, citrate, histidine, maleate, phosphate, succinate, tartrate and TRIS. Where a composition comprises an aluminum salt, it is preferred to use a histidine buffer [WO03/009869]. Histidine may be added to the composition in the form of the amino acid itself, preferably L-histidine, or in the form of a salt. The concentration of histidine in the composition may be typically of at least 1 μM up to 1M. In an embodiment, the concentration is at least 1 mM (e.g. at least 2 mM, 3 mM, 4 mM, 5 mM etc.) up to 250 mM (e.g. at most 200 mM, 150 mM, 100 mM, 50 mM, 40 mM, 30 mM, 20 mM, 10 mM, etc.). Preferably the concentration of histidine is between 2 mM and 20 mM (e.g. between 5 mM and 15 mM), most preferably it is about 10 mM.

In an embodiment, the reconstituted vaccine composition of the invention has a pharmaceutically acceptable osmolality to avoid cell distortion or lysis. A pharmaceutically acceptable osmolality will generally mean that solutions will have an osmolality, which is approximately isotonic or mildly hypertonic. In an embodiment, the reconstituted vaccine composition will have an osmolality in the range of 250 to 750 mOsm/kg, for example, the osmolality may be in the range of 250 to 550 mOsm/kg, such as in the range of 280 to 500 mOsm/kg. Suitably the liquid formulation of MenB antigens of the present invention will be slightly hypotonic, for example will have osmolality of about 210 mOsm/Kg, so that the reconstituted vaccine will reach the desired isotonicity or mild hypertonicity, as said above. Osmolality may be measured according to techniques known in the art, such as by means of a commercially available osmometer, for example the Advanced® Model 2020 available from Advanced Instruments Inc. (USA).

In a preferred embodiment, the immunogenic composition against N. meningitidis serogroups A, C, W, Y in the reconstituted vaccine of this invention, which is formulated as a solid, for instance as a lyophilized (freeze-dried) product, comprises a pharmaceutically acceptable bulking agent, such as a sugar.

The term “bulking agent” refers herein to an excipient compound or to mixtures of compounds that, when added to a solution intended for lyophilization, make up the bulk lyophilized product. Non-limiting examples of bulking agents are sucrose, mannitol, trehalose and mixtures thereof; preferably, the bulking agent is sucrose. In an embodiment, the bulking agent, preferably sucrose, is added to the capsular saccharide conjugates at a concentration lower than about 6% by weight with respect to the total volume of the formulation of the Men A, C, W, Y antigens before lyophilization (referred to in the following as “w/v”). In an embodiment, the bulking agent, preferably sucrose, is added to the capsular saccharide conjugates at a concentration lower than about 5% by weight with respect to the total volume of the formulation of the Men A, C, W, Y antigens before lyophilization. In an embodiment, the bulking agent, preferably sucrose, is added at a concentration of about 2% w/v, about 3% w/v, about 4% w/v, about 5% w/v, preferably of about 3% w/v, in the formulation before lyophilization, indicated in the following also as pre-lyo bulk formulation. In further aspects, the concentration of bulking agent, preferably sucrose, in the pre-lyo bulk formulation of Men A, C, W, Y antigens may range between 2% w/v and 5.5% w/v, between 2% w/v and 5% w/v, between 2% w/v and 4% w/v, or between 2.5% w/v and 3.5% w/v. In further aspects, the concentration of bulking agent, preferably sucrose, in the pre-lyo bulk formulation of Men A, C, W, Y antigens is 2% w/v or 3% w/v.

In a further preferred embodiment, the immunogenic composition against N. meningitidis serogroups A, C, W, Y in the reconstituted vaccine of this invention comprises a buffering agent, for example a phosphate buffer at pH of about 7.2. This phosphate buffer may comprise potassium monobasic phosphate, to which potassium dibasic phosphate is added.

A bulking agent and/or a buffering agent is preferably added to the composition and mixed to the antigens before lyophilization. In an embodiment, a phosphate buffer at a concentration of at least about 6 mM, at least 10 mM, or at least 40 mM, is added to the pre-lyophilization formulation.

As shown in the experimental part below, the liquid formulation of Men B antigens and the solid formulation of the Men A, C, W135, and Y antigens as described herein allow to obtain not only a safe, stable and easy to use vaccine product, but also to provide the vaccine in an effective formulation and for long-term storage. Characteristics of the present formulations include, but are not limited to, chemical stability of the immunogenic composition (e.g. proteolysis or fragmentation of proteins), physical/thermal stability of the immunogenic composition (e.g., aggregation, precipitation, adsorption), compatibility of the immunogenic composition with the container/closure system, interactions between immunogenic composition and inactive ingredients (e.g. buffers, salts, excipients, cryoprotectants), the manufacturing process, the dosage form (e.g., lyophilized, liquid), the environmental conditions encountered during shipping, storage and handling (e.g., temperature, humidity, shear forces), and the length of time between manufacture and usage.

Immunogenic compositions comprise an immunologically effective amount of the protein or conjugate of the invention, as well as any other components. By “immunologically effective amount”, it is meant that the administration of that amount to an individual, either as a single dose or as part of a series is effective for treatment or prevention. This amount varies depending on the health and physical condition of the individual to be treated, age, the degree of protection desired, the formulation of the vaccine and other relevant factors. It is expected that the amount will fall in a relatively broad range that can be determined through routine trials.

In an embodiment, the present immunogenic composition in liquid formulation against N. meningitidis serogroup B infections comprises 40-60 μg/ml, e.g. 50 μg/ml, of the OMV antigen. In an embodiment, this immunogenic composition comprises 50-150 μg/ml, e.g. 100 μg/ml, of each protein antigen NHBA, fHbp, and NadA. In an embodiment, this immunogenic composition comprises 100-400 μg/ml, e.g. 100, 200, 300, or 400 μg/ml of the fusion fHbp polypeptide. In another embodiment, this immunogenic composition comprises 100-200 μg/ml of the fusion fHbp polypeptide, e.g. of the mutant v1.13 fHbp polypeptide.

In an embodiment, the immunogenic composition in solid form against N. meningitidis serogroups ACWY infections in the present reconstituted vaccine comprises 5-15 μg/ml, e.g. 10 g, of each MenC, MenW, and MenY saccharide. In an embodiment, this immunogenic composition comprises 5-15 g, e.g. 20 μg/ml, of the MenA saccharide.

As described above, the immunogenic composition in solid form of this invention preferably comprises the Men A, C, W135 and Y antigen in the form of conjugates, wherein the Men A, C, W135, and Y capsular saccharides and/or oligosaccharides thereof are conjugated to CRM₁₉₇ carrier, including recombinant CRM₁₉₇ carrier (rCRM₁₉₇). In an embodiment, a unit dose of 0.5 mL of the present reconstituted vaccine comprises 10 g of the Men A saccharide conjugated to from 12.5 to 33.3 g of CRM₁₉₇, 5 g of the Men C saccharide conjugated to from 6.3 to 12.5 g of CRM₁₉₇, 5 g of Men W135 saccharide conjugated to from 3.3 to 10.0 g of CRM₁₉₇, 5 g of the Men Y saccharide conjugated to from 3.3 to 10.0 g of CRM₁₉₇.

In an embodiment, the reconstituted vaccine composition against N. meningitidis serogroups A, B, C, W135, and Y of the present invention comprises OMVs at 40-60 μg/ml, e.g. 50 μg/ml; each of the NHBA antigen, NadA antigen, and fHbp antigen at 50-150 μg/ml, e.g. 100 μg/ml; the fusion fHbp polypeptide at 100-400 μg/ml, e.g. 100, 200, 300, or 400 μg/ml; each of Men C, Men W135 and Men Y at 5-15 μg/ml, e.g. 10 μg/ml; Men A at 5-15 μg/ml, e.g. 10 μg/ml; an absorbing agent, e.g. Alum, at 2 to 5 μg/ml, e.g. 3 μg/ml; and a tonicity modifying agent, e.g. sodium chloride, at 2.0 to 3.5 μg/ml, e.g. 2.8 μg/ml.

In an embodiment, a dose of 0.5 ml of the present reconstituted vaccine comprises: 10 μg of Men saccharide, 5 μg of each of the Men C, Men W135 and Men Y saccharides, 25 μg of OMVs, 50 g of each of the rp287-953, rp936-741 and rp961c antigens, 50 μg of fHbp231.13, 1.4 mg of sodium chloride, 27.5 mg of sucrose, 0.54 mg of potassium phosphate salts, 0.776 mg of histidine, 1.54 mg of Aluminum hydroxide, and water for injection up to 0.5 ml dose. In other embodiments, the same 0.5 dose comprises a higher amount of fHbp231.13 of 100 μg or of 200 μg, instead of 50 μg, and 0.54 to 0.90 mg of potassium phosphate salts.

### Kit and Reconstituted Vaccine Composition

Subject of this invention is a kit comprising (i) a first container comprising an immunogenic composition against N. meningitidis serogroup B as described above, in a liquid formulation; and (ii) a second container comprising an immunogenic composition against N. meningitidis serogroups A, C, W135, and Y as described above, in a solid form. In the present kit, the first container and the second container can be containers separated from each other or they can form together a multi-container with separated parts for the liquid component and for the solid one.

In the present invention the liquid immunogenic composition in the first container is used to reconstitute the solid immunogenic composition in the second container, thus forming a vaccine composition comprising all antigens of both immunogenic compositions prior to administration. In an embodiment the reconstituted vaccine composition is a suspension. In an embodiment the reconstituted vaccine composition comprises a suspension of the solid immunogenic composition against N. meningitidis serogroups A, C, W135, and Y as described herein in the liquid immunogenic composition against N. meningitidis serogroup B as described herein.

In a preferred embodiment, the kit of this invention comprises (i) a pre-filled syringe as the first container and (ii) a vial as the second container.

In a particular embodiment, containers in the present kit are siliconized, to improve consistency of withdrawal of the vaccine upon reconstitution. Where an immunogenic composition of the invention is presented in a vial, this is preferably made of a glass or plastic material, more preferably this is made of siliconized glass or plastic material.

The vial is preferably sterilized before the composition is added to it. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass. A vial can have a cap or a stopper (e.g. a Luer-Lok™) adapted such that a pre-filled syringe, equipped with a needle, can be inserted into the cap, the contents of the syringe can be expelled into the vial (e.g. to reconstitute lyophilised material therein), and the contents of the vial can be withdrawn back into the syringe. After removal of the syringe from the vial, the composition can be administered to a patient. The cap is preferably located inside a seal or cover, such that the seal or cover has to be removed before the cap can be accessed.

In an embodiment, a same needle is used both for the reconstitution and for parenteral administration. In another embodiment, a needle used for the reconstitution is replacement by another needle for administration of the reconstituted vaccine.

As showed by the results in the experimental part below, the liquid formulation of the Men B antigens composition guarantees that all relevant quality requirements of the final vaccine are met, in particular in terms of pH and osmolality of the vaccine composition, integrity and immunogenicity of each and all antigens. Moreover, the present liquid formulation of MenB antigens has shown an optimal percentage of adsorption of all antigens onto the adsorbing agent, even following to reconstitution of the solid formulation of MenACWY antigens.

### Administration

The present vaccine composition will generally be administered directly to a patient by an appropriate route, for example by parenteral injection, e.g. intramuscularly. Intramuscular administration to the thigh or the upper arm is preferred. Injection may be via a needle (e.g. a hypodermic needle), but needle-free injection may alternatively be used.

The reconstituted vaccine composition of this invention may be administered to patients in unit doses, ranging between 0.1 and 1 ml, e.g. 0.5 ml. A typical intramuscular dose is of about 0.5 ml.

Neisserial infections affect various areas of the body and so the compositions of the invention may be prepared in various forms. Compositions suitable for parenteral injection are most preferred.

The invention may be used to elicit systemic and/or mucosal immunity.

As used herein, a ‘dose’ of the composition is a volume of the composition suitable for administration to a subject as a single immunisation. Human vaccines are typically administered in a dosage volume of about 0.5 ml, although fractional doses may be administered (e.g., to children).

The composition may further be provided in a ‘multidose’ kit, i.e., a single container containing sufficient composition for multiple immunisations. Multidoses may include a preservative, or the multidose container may have an aseptic adaptor for removal of individual doses of the composition.

Administration can involve a single dose schedule or a multiple dose schedule. In this latter case, suitable intervals between priming doses can be routinely determined e.g. between 4-16 weeks, such as one month or two months.

The subject who is immunized is a human being, who may be any age e.g. 0-12 months old, 1-5 years old, 5-18 years old, 18-55 years old, or more than 55 years old. Preferably, the subject who is immunized is an adolescent (e.g. 12-18 years old) or an adult (18 years or older).

Optionally, the subject is an adolescent or adult who has been immunized against N. meningitidis in childhood (e.g. before 12 years of age), and who receives a booster dose of an immunogenic composition according to the invention.

### Use of the Immunogenic Composition and of the Reconstituted Vaccine of the Invention

The Men B immunogenic composition of the invention is suitable for use in medicine, and in particular can be used to immunize a mammal against infection and/or disease caused by Neisseria meningitides serogroup B, such that recipients of the immunogenic composition mount an immune response, which provides protection against infection by, and/or disease due to Neisseria meningitidis bacteria.

The reconstituted vaccine composition as described above is useful for immunizing a mammal against meningococcal A, B, C, W and/or Y infection or disease.

Therefore, immunogenic composition and reconstituted vaccine according to the invention are used in prophylactic methods for immunizing subjects against infection and/or disease caused by Neisseria meningitidis The immunogenic composition and the reconstituted vaccine may also be used in therapeutic methods (i.e. to treat Neisseria meningitidis infection).

The invention also provides a method for raising an immune response in vivo against Neisseria meningitidis infection in a mammal, comprising administering an immunogenic composition of the invention or the reconstituted vaccine to the mammal.

The immune response is preferably protective and preferably involves antibodies and/or cell-mediated immunity. Preferably, the immune response is a bactericidal antibody response. The method may raise a booster response. By raising an in vivo immune response, the mammal can be protected against Neisserial disease (in particular meningococcal infection).

The invention also provides a method for protecting a mammal against a Neisserial (e.g. meningococcal) infection, comprising administering to the mammal an immunogenic composition or the reconstituted vaccine of the invention.

The immunological composition of the invention is preferably formulated as vaccine products, which are suitable for therapeutic (i.e. to treat an infection) or prophylactic (i.e. to prevent an infection) use. Vaccines are typically prophylactic.

The mammal is preferably a human. The human may be an adult, an adolescent or a child (e.g. a toddler or infant). A vaccine intended for children may also be administered to adults e.g. to assess safety, dosage, immunogenicity, etc.

The uses and methods are particularly useful for preventing/treating diseases including, but not limited to, meningitis (particularly bacterial, such as meningococcal, meningitis) and bacteremia. For instance, they are suitable for active immunisation of individuals against invasive meningococcal disease caused by N. meningitidis, preferably caused by N. meningitidis serogroups A, B, C, W, or Y.

Protection against N. meningitidis can be measured epidemiologically e.g. in a clinical trial, but it is convenient to use an indirect measure to confirm that an immunogenic composition elicits a serum bactericidal antibody (SBA) response in recipients. In the SBA assay, sera from recipients of the composition are incubated with target bacteria (in the present invention, N. meningitidis) in the presence of complement (preferably human complement, although baby rabbit complement is often used instead) and killing of the bacteria is assessed at various dilutions of the sera to determine SBA activity. Results observed in the SBA assay can be reinforced by carrying out a competitive SBA assay to provide further indirect evidence of the immunogenic activity of antigen(s) of interest. In the competitive SBA assay, sera from recipients of the immunogenic composition containing the antigen(s) are pre-incubated with said antigen(s), and subsequently incubated with target bacteria in the presence of human complement. Killing of the bacteria is then assessed, and they will be reduced or abolished if bactericidal antibodies in the recipients' sera bind to the antigens of interested during the pre-incubation phase and are therefore not available to bind to surface antigen on the bacteria.

It is not necessary that the composition should protect against each and every strain of N. meningitidis, or that each and every recipient of the composition must be protected. Such universal protection is not the normal standard in this field. Rather, protection is normally assessed against a panel of reference laboratory strains, often selected on a country-by-country basis and perhaps varying with time and is measured across a population of recipients.

Preferred compositions of the invention can confer an antibody titer in a patient that is superior to the criterion for seroprotection for each antigenic component for an acceptable percentage of human subjects. Antigens with an associated antibody titer above which a host is considered to be seroconverted against the antigen are well known, and such titers are published by organizations, such as WHO. Preferably more than 80% of a statistically significant sample of subjects is seroconverted, more preferably more than 90%, still more preferably more than 93% and most preferably 96-100%.

Immunogenic compositions comprise an immunologically effective amount of immunogen, as well as any other of other specified components, as needed.

By ‘immunologically effective amount’, it is meant that the administration of that amount to an individual, either in a single dose or as part of a series, is effective for treatment or prevention.

The term “prevention” means that the progression of the disease is reduced and/or eliminated, or that the onset of the disease is eliminated. For example, the immune system of a subject may be primed (e.g. by vaccination) to trigger an immune response and repel infection such that the onset of the disease is eliminated.

A vaccinated subject may thus get infected but is better able to repel the infection than a control subject. This amount varies depending upon the health and physical condition of the individual to be treated, age, the taxonomic group of the individual to be treated (e.g. non-human primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

The composition may be administered in conjunction with other immunoregulatory agents.

### Vaccine Efficacy

A reconstituted vaccine composition for use in the present invention preferably has a vaccine efficacy against each strain of N. meningitidis of at least 10% e.g. ≥20%, ≥30%, ≥40%, ≥50%, ≥60%, ≥70%, ≥80%, ≥85%, ≥90%, or more.

Vaccine efficacy is determined by the reduction in relative risk of developing meningococcal disease in subjects who receive a composition according to the invention compared to subjects who do not receive such a composition (e.g. are non-immunized or who receive a placebo or negative control). Thus, the incidence of meningococcal disease in a population which has been immunized according to the invention is compared to the incidence in a control population who has not been immunized according to the invention to give relative risk and vaccine efficacy is 100% minus this figure.

Vaccine efficacy is determined for a population rather than for an individual. Thus, it is a useful epidemiologic tool but does not predict individual protection. For instance, an individual subject might be exposed to a very large inoculum of the infecting agent or might have other risk factors which make them more subject to infection, but this does not negate the validity or utility of the efficacy measure. The size of a population which is immunized according to the invention, and for which vaccine efficacy is measured, is ideally at least 100 and maybe higher e.g. at least 500 subjects. The size of the control group should also be at least 100 e.g. at least 500.

All references or patent applications cited within this specification are incorporated herein by reference.

Aspects of the invention are summarized in the following numbered paragraphs:
1. An immunogenic composition against N. meningitidis serogroup B, comprising: a meningococcal NHBA antigen, a meningococcal NadA antigen, a meningococcal fHbp antigen, a meningococcal outer membrane vesicles (OMVs), and a fusion polypeptide of meningococcal fHbp polypeptides, wherein said composition is a liquid formulation comprising an adsorbing agent for said antigens and polypeptides and one or more pharmaceutically acceptable tonicity modifying agents.
2. The immunogenic composition of paragraph 1, wherein said tonicity modifying agents are selected from sodium chloride, sorbitol, sucrose and mixtures thereof.
3. The immunogenic composition of any one of the paragraphs 1 or 2, wherein said liquid formulation comprises an aqueous solution of sodium chloride and sucrose.
4. The immunogenic composition of any one of the preceding paragraphs, wherein said liquid formulation has a concentration of sodium chloride≤3.8 mg/ml.
5. The immunogenic composition of any one of the preceding paragraphs, wherein said liquid formulation has a concentration of sodium chloride comprised between 1 mg/ml and 3.5 mg/ml, e.g. 2.8 mg/ml.
6. The immunogenic composition of any one of the preceding paragraphs, having a concentration of said fusion polypeptide of meningococcal fHbp polypeptides of 100-400 μg/ml.
7. The immunogenic composition of any one of the preceding paragraphs, wherein said liquid formulation has a concentration of sucrose comprised between 2.5 and 3.5% w/v, e.g. 3% w/v.
8. The immunogenic composition of any one of the preceding paragraphs, wherein said fusion polypeptide of meningococcal fHbp polypeptides comprises v1, v2 and v3 meningococcal fHbp polypeptides, wherein the variant fHbp sequences are in the order v2-v3-v1 from N- to C-terminus.
9. The immunogenic composition of paragraph 8, wherein the v1 fHbp polypeptide is a mutant v1.13 fHbp polypeptide.
10. The immunogenic composition of paragraph 9, wherein said mutant v1.13 fHbp polypeptide has an amino acid sequence differing from SEQ ID NO: 16 by at least one or more of the substitutions E211A, S216R and E232A.
11. The immunogenic composition of any one of the preceding paragraphs, wherein said meningococcal NHBA antigen and said meningococcal fHbp antigen are fusion proteins with meningococcal accessory proteins, e.g. a NHBA-GNA1030 fusion protein and a GNA2091-fHbp fusion protein.
12. The immunogenic composition of any one of the preceding paragraphs, wherein said adsorbing agent comprises a compound containing aluminium selected from aluminium hydroxide (Alum), aluminium salts and mixtures thereof.
13. The immunogenic composition of any one of the preceding paragraphs, wherein said adsorbing agent is Alum.
14. The immunogenic composition of any one of the preceding paragraphs, further comprising a buffering agent, preferably a histidine buffer.
15. The immunogenic composition of any one of the preceding paragraphs, wherein said OMVs are present at 50 μg/ml, each of said NHBA antigen, NadA antigen, and fHbp antigen are present at 100 μg/ml, said fusion polypeptide is present at 100 or 200 μg/ml, said sucrose is present at 2.5 to 3.5% w/v, e.g. 3% w/v, said buffer is prepared at pH 6.1-6.3, Alum is present at 2 to 5 mg/ml, e.g. 3 mg/ml and sodium chloride is present at 2.0 to 3.5 mg/ml, e.g. 2.8 mg/ml.
16. A reconstituted vaccine composition comprising an immunogenic composition against N. meningitidis serogroups A, C, W135, and Y, comprising capsular saccharides antigens of the meningococcal serogroups A, C, W135, and Y that are conjugated to carrier protein(s) in a solid form (e.g. freeze-dried), reconstituted with the immunogenic composition against N. meningitidis serogroup B in liquid form as defined in paragraphs 1-15.
17. The reconstituted vaccine of paragraph 16, wherein said immunogenic composition against N. meningitidis serogroups A, C, W135, and Y comprises a bulking agent, optionally selected from the group consisting of sucrose, mannitol, trehalose and mixtures thereof; preferably is sucrose.
18. The reconstituted vaccine of any one of paragraphs 16 or 17, wherein said an immunogenic composition against N. meningitidis serogroups A, C, W135, and Y, further comprises a buffering agent.
19. The reconstituted vaccine of paragraph 18, wherein said buffering agent is a phosphate buffer comprising potassium monobasic phosphate, to which potassium dibasic phosphate is added.
20. The reconstituted vaccine of any one of paragraphs 16 to 19, wherein said capsular saccharides antigens of the meningococcal serogroups A, C, W135, and Y are conjugated to CRM₁₉₇ as carrier protein.
21. The reconstituted vaccine of any one of paragraphs 16 to 20, comprising sodium chloride at a concentration of 2 to 3.5 mg/ml (e.g. 2.8 mg/ml).
22. A reconstituted vaccine composition comprising a suspension of an immunogenic composition against N. meningitidis serogroups A, C, W135, and Y, comprising capsular saccharides antigens of the meningococcal serogroups A, C, W135, and Y that are conjugated to carrier protein(s) in a solid form (e.g. freeze-dried), in the liquid immunogenic composition against N. meningitidis serogroup B as defined in paragraphs 1-15.
23. The reconstituted vaccine composition paragraph 22, wherein said immunogenic composition against N. meningitidis serogroups A, C, W135, and Y is as defined in paragraphs 16-21.
24. A kit comprising (i) a first container comprising the immunogenic composition against N. meningitidis serogroup B in a liquid form of any one of paragraphs 1 to 15; and (ii) a second container comprising an immunogenic composition against N. meningitidis serogroups A, C, W135, and Y, comprising capsular saccharides antigens of the meningococcal serogroups A, C, W135, and Y that are conjugated to carrier protein(s) in a solid form (e.g. freeze-dried), wherein said first container and said second container are containers separated from each other or they formed together a multi-container.
25. The kit of paragraph 24, wherein said first container is a prefilled syringe and said second container is a vial.
26. The kit of any one of paragraphs 24 or 25, wherein said containers or said multi-container are siliconized.
27. An immunogenic composition of any of paragraphs 1 to 15, or a reconstituted vaccine of any of paragraphs 16 to 21, for use as a vaccine.
28. An immunogenic composition of any of paragraphs 1 to 15, or a reconstituted vaccine of any of paragraphs 16 to 21, for use in a method for the prophylaxis or treatment of N. meningitidis infections in a mammal, e.g. a human.
29. A method for preparing the reconstituted vaccine against N. meningitidis serogroups A, B, C, W135, and Y of any of paragraphs 16 to 21, comprising a step of reconstituting an immunogenic composition against N. meningitidis serogroups A C, W135, and Y in solid form as in paragraphs 16 to 21, with an immunogenic composition against N. meningitidis serogroup B in liquid form as defined in any of paragraphs 1 to 15.
30. A method for the treatment or prevention of a meningococcal infection or disease caused by N. meningitidis in a subject in need thereof comprising administering to said subject a therapeutically effective amount of an immunogenic composition according to any of paragraphs 1 to 15 or a reconstituted vaccine according to any of paragraphs 16 to 21.
31. A method of inducing an immune response to N. meningitidis in a subject, the method comprising administering a therapeutically or prophylactically effective amount of an immunogenic composition according to any of paragraphs 1 to 15 or of a reconstituted vaccine according to any of paragraphs 16 to 21.
32. Use of an immunogenic composition according to any of paragraphs 1 to 15 or of a reconstituted vaccine according to any of paragraphs 16 to 21 in the manufacture of a medicament for the treatment or prevention of a meningococcal infection or disease caused by N. meningitidis.

It will be understood that the invention is described above by way of example only and modifications may be made whilst remaining within the scope and spirit of the invention.

### DESCRIPTION OF THE SEQUENCE LISTING

[fHbp protein from strain MC58]
SEQ ID NO: 1
CSSGGGGVAADIGAGLADALTAPLDHKDKGLQSLTLDQSVRKNEKLKLAAQGAEKTYGNGDSLNTGKLKNDKVSRF
DFIRQIEVDGQLITLESGEFQVYKQSHSALTAFQTEQIQDSEHSGKMVAKRQFRIGDIAGEHTSFDKLPEGGRATYR
GTAFGSDDAGGKLTYTIDFAAKQGNGKIEHLKSPELNVDLAAADIKPDGKRHAVISGSVLYNQAEKGSYSLGIFGGK
AQEVAGSAEVKTVNGIRHIGLAAKQ
[fHbp protein from strain 961-5945]
SEQ ID NO: 2
CSSGGGGVAADIGAGLADALTAPLDHKDKSLQSLTLDQSVRKNEKLKLAAQGAEKTYGNGDSLNTGKLKNDKVSRF
DFIRQIEVDGQLITLESGEFQIYKQDHSAVVALQIEKINNPDKIDSLINQRSFLVSGLGGEHTAFNQLPDGKAEYHGK
AFSSDDAGGKLTYTIDFAAKQGHGKIEHLKTPEQNVELAAAELKADEKSHAVILGDTRYGSEEKGTYHLALFGDRAQ
EIAGSATVKIGEKVHEIGIAGKQ
[fHbp from strain M1239]
SEQ ID NO: 3
CSSGGGGSGGGGVAADIGTGLADALTAPLDHKDKGLKSLTLEDSIPQNGTLTLSAQGAEKTFKAGDKDNSLNTGKL
KNDKISRFDFVQKIEVDGQTITLASGEFQIYKQNHSAVVALQIEKINNPDKTDSLINQRSFLVSGLGGEHTAFNQLPG
GKAEYHGKAFSSDDPNGRLHYSIDFTKKQGYGRIEHLKTLEQNVELAAAELKADEKSHAVILGDTRYGSEEKGTYHL
ALFGDRAQEIAGSATVKIGEKVHEIGIAGKQ
[NHBA wild-type from strain MC58]
SEQ ID NO: 4
MFKRSVIAMACIFALSACGGGGGGSPDVKSADTLSKPAAPVVSEKETEAKEDAPQAGSQGQGAPSAQGSQDMAAV
SEENTGNGGAVTADNPKNEDEVAQNDMPQNAAGTDSSTPNHTPDPNMLAGNMENQATDAGESSQPANQPDMAN
AADGMQGDDPSAGGQNAGNTAAQGANQAGNNQAAGSSDPIPASNPAPANGGSNFGRVDLANGVLIDGPSQNITL
THCKGDSCSGNNFLDEEVQLKSEFEKLSDADKISNYKKDGKNDKFVGLVADSVQMKGINQYIIFYKPKPTSFARFRR
SARSRRSLPAEMPLIPVNQADTLIVDGEAVSLTGHSGNIFAPEGNYRYLTYGAEKLPGGSYALRVQGEPAKGEMLAG
AAVYNGEVLHFHTENGRPYPTRGRFAAKVDFGSKSVDGIIDSGDDLHMGTQKFKAAIDGNGFKGTWTENGSGDVS
GKFYGPAGEEVAGKYSYRPTDAEKGGFGVFAGKKEQD
[NadA from strain MC58]
SEQ ID NO: 5
MSMKHFPSKVLTTAILATFCSGALAATSDDDVKKAATVAIVAAYNNGQEINGFKAGETIYDIGEDGTITQKDATAAD
VEADDFKGLGLKKVVTNLTKTVNENKQNVDAKVKAAESEIEKLTTKLADTDAALADTDAALDETTNALNKLGENITT
FAEETKTNIVKIDEKLEAVADTVDKHAEAFNDIADSLDETNTKADEAVKTANEAKQTAEETKQNVDAKVKAAETAAG
KAEAAAGTANTAADKAEAVAAKVTDIKADIATNKADIAKNSARIDSLDKNVANLRKETRQGLAEQAALSGLFQPYNV
GRFNVTAAVGGYKSESAVAIGTGFRFTENFAAKAGVAVGTSSGSSAAYHVGVNYEW
[fusion protein of fHbp]
SEQ ID NO: 6
VAADIGAGLADALTAPLDHKDKGLQSLTLDQSVRKNEKLKLAAQGAEKTYGNGDSLNTGKLKNDKVSRFDFIRQIEV
DGQLITLESGEFQVYKQSHSALTAFQTEQIQDSEHSGKMVAKRQFRIGDIAGEHTSFDKLPEGGRATYRGTAFGSD
DAGGKLTYTIDFAAKQGNGKIEHLKSPELNVDLAAADIKPDGKRHAVISGSVLYNQAEKGSYSLGIFGGKAQEVAGS
AEVKTVNGIRHIGLAAKQ
[GNA2091-fHbp fusion protein]
SEQ ID NO: 7
MVSAVIGSAAVGAKSAVDRRTTGAQTDDNVMALRIETTARSYLRQNNQTKGYTPQISVVGYDRHLLLLGQVATEGE
KQFVGQIARSEQAAEGVYNYITVASLPRTAGDIAGDTWNTSKVRATLLGISPATRARVKIVTYGNVTYVMGILTPEE
QAQITQKVSTTVGVQKVITLYQNYVQRGSGGGGVAADIGAGLADALTAPLDHKDKGLQSLTLDQSVRKNEKLKLAA
QGAEKTYGNGDSLNTGKLKNDKVSRFDFIRQIEVDGQLITLESGEFQVYKQSHSALTAFQTEQIQDSEHSGKMVAK
RQFRIGDIAGEHTSFDKLPEGGRATYRGTAFGSDDAGGKLTYTIDFAAKQGNGKIEHLKSPELNVDLAAADIKPDGK
RHAVISGSVLYNQAEKGSYSLGIFGGKAQEVAGSAEVKTVNGIRHIGLAAKQ
[DG variant of NHBA]
SEQ ID NO: 8
SPDVKSADTLSKPAAPVVSEKETEAKEDAPQAGSQGQGAPSAQGGQDMAAVSEENTGNGGAAATDKPKNEDEGA
QNDMPQNAADTDSLTPNHTPASNMPAGNMENQAPDAGESEQPANQPDMANTADGMQGDDPSAGGENAGNTAA
QGTNQAENNQTAGSQNPASSTNPSATNSGGDFGRTNVGNSVVIDGPSQNITLTHCKGDSCSGNNFLDEEVQLKSE
FEKLSDADKISNYKKDGKNDGKNDKFVGLVADSVQMKGINQYIIFYKPKPTSFARFRRSARSRRSLPAEMPLIPVNQA
DTLIVDGEAVSLTGHSGNIFAPEGNYRYLTYGAEKLPGGSYALRVQGEPSKGEMLAGTAVYNGEVLHFHTENGRPSP
SRGRFAAKVDFGSKSVDGIIDSGDGLHMGTQKFKAAIDGNGFKGTWTENGGGDVSGKFYGPAGEEVAGKYSYRPT
DAEKGGFGVFAGKKEQD
[NHBA-GNA1030 fusion]
SEQ ID NO: 9
MASPDVKSADTLSKPAAPVVSEKETEAKEDAPQAGSQGQGAPSAQGGQDMAAVSEENTGNGGAAATDKPKNEDE
GAQNDMPQNAADTDSLTPNHTPASNMPAGNMENQAPDAGESEQPANQPDMANTADGMQGDDPSAGGENAGNT
AAQGTNQAENNQTAGSQNPASSTNPSATNSGGDFGRTNVGNSVVIDGPSQNITLTHCKGDSCSGNNFLDEEVQLK
SEFEKLSDADKISNYKKDGKNDGKNDKFVGLVADSVQMKGINQYIIFYKPKPTSFARFRRSARSRRSLPAEMPLIPVN
QADTLIVDGEAVSLTGHSGNIFAPEGNYRYLTYGAEKLPGGSYALRVQGEPSKGEMLAGTAVYNGEVLHFHTENGR
PSPSRGRFAAKVDFGSKSVDGIIDSGDGLHMGTQKFKAAIDGNGFKGTWTENGGGDVSGKFYGPAGEEVAGKYSY
RPTDAEKGGFGVFAGKKEQDGSGGGGATYKVDEYHANARFAIDHFNTSTNVGGFYGLTGSVEFDQAKRDGKIDITI
PVANLQSGSQHFTDHLKSADIFDAAQYPDIRFVSTKFNFNGKKLVSVDGNLTMHGKTAPVKLKAEKFNCYQSPMAK
TEVCGGDFSTTIDRTKWGVDYLVNVGMTKSVRIDIQIEAAKQ
[NadA fragment]
SEQ ID NO: 10
ATNDDDVKKAATVAIAAAYNNGQEINGFKAGETIYDIDEDGTITKKDATAADVEADDFKGLGLKKVVTNLTKTVNEN
KQNVDAKVKAAESEIEKLTTKLADTDAALADTDAALDATTNALNKLGENITTFAEETKTNIVKIDEKLEAVADTVDKH
AEAFNDIADSLDETNTKADEAVKTANEAKQTAEETKQNVDAKVKAAETAAGKAEAAAGTANTAADKAEAVAAKVTD
IKADIATNKDNIAKKANSADVYTREESDSKFVRIDGLNATTEKLDTRLASAEKSIADHDTRLNGLDKTVSDLRKETRQ
GLAEQAALSGLFQPYNVG
[fHbp sequence]
SEQ ID NO: 11
CSSGSGSGGGGVAADIGTGLADALTAPLDHKDKGLKSLTLEDSISQNGTLTLSAQGAEKTFKVGDKDNSLNTGKLK
NDKISRFDFVQKIEVDGQTITLASGEFQIYKQDHSAVVALQIEKINNPDKIDSLINQRSFLVSGLGGEHTAFNQLPSG
KAEYHGKAFSSDDAGGKLTYTIDFAAKQGHGKIEHLKTPEQNVELASAELKADEKSHAVILGDTRYGSEEKGTYHLA
LFGDRAQEIAGSATVKIREKVHEIGIAGKQ
[fHbp sequence]
SEQ ID NO: 12
CSSGGGGSGGGGVTADIGTGLADALTAPLDHKDKGLKSLTLEDSISQNGTLTLSAQGAEKTYGNGDSLNTGKLKND
KVSRFDFIRQIEVDGQLITLESGEFQVYKQSHSALTALQTEQEQDPEHSEKMVAKRRFRIGDIAGEHTSFDKLPKDV
MATYRGTAFGSDDAGGKLTYTIDFAAKQGHGKIEHLKSPELNVDLAVAYIKPDEKHHAVISGSVLYNQDEKGSYSLG
IFGEKAQEVAGSAEVETANGIHHIGLAAKQ
[GNA1030^{NL}]
SEQ ID NO: 13
ATYKVDEYHANARFAIDHFNTSTNVGGFYGLTGSVEFDQAKRDGKIDITIPIANLQSGSQHFTDHLKSADIFDAAQY
PDIRFVSTKFNFNGKKLVSVDGNLTMHGKTAPVKLKAEKFNCYQSPMEKTEVCGGDFSTTIDRTKWGMDYLVNVG
MTKSVRIDIQIEAAKQ
[GNA2091^{NL}]
SEQ ID NO: 14
VSAVIGSAAVGAKSAVDRRTTGAQTDDNVMALRIETTARSYLRQNNQTKGYTPQISVVGYNRHLLLLGQVATEGEK
QFVGQIARSEQAAEGVYNYITVASLPRTAGDIAGDTWNTSKVRATLLGISPATQARVKIVTYGNVTYVMGILTPEEQ
AQITQKVSTTVGVQKVITLYQNYVQR
[v1.13 mature polypeptide from strain M982]
SEQ ID NO: 15
CSSGGGGVAADIGAGLADALTAPLDHKDKGLQSLTLDQSVRKNEKLKLAAQGAEKTYGNGDSLNTGKLKNDKVSRF
DFIRQIEVDGKLITLESGEFQVYKQSHSALTALQTEQVQDSEDSGKMVAKRQFRIGDIAGEHTSFDKLPKGGSATYR
GTAFGSDDAGGKLTYTIDFAAKQGHGKIEHLKSPELNVELATAYIKPDEKRHAVISGSVLYNQDEKGSYSLGIFGGQA
QEVAGSAEVETANGIHHIGLAAKQ
[v1.13 ΔG]
SEQ ID NO: 16
VAADIGAGLADALTAPLDHKDKGLQSLTLDQSVRKNEKLKLAAQGAEKTYGNGDSLNTGKLKNDKVSRFDFIRQIEV
DGKLITLESGEFQVYKQSHSALTALQTEQVQDSEDSGKMVAKRQFRIGDIAGEHTSFDKLPKGGSATYRGTAFGSD
DAGGKLTYTIDFAAKQGHGKIEHLKSPELNVELATAYIKPDEKRHAVISGSVLYNQDEKGSYSLGIFGGQAQEVAGSA
EVETANGIHHIGLAAKQ
[v1.13 ΔG (E211A/E232A)]
SEQ ID NO: 17
VAADIGAGLADALTAPLDHKDKGLQSLTLDQSVRKNEKLKLAAQGAEKTYGNGDSLNTGKLKNDKVSRFDFIRQIEV
DGKLITLESGEFQVYKQSHSALTALQTEQVQDSEDSGKMVAKRQFRIGDIAGEHTSFDKLPKGGSATYRGTAFGSD
DAGGKLTYTIDFAAKQGHGKIEHLKSPELNVELATAYIKPDEKRHAVISGSVLYNQDAKGSYSLGIFGGQAQEVAGS
AAVETANGIHHIGLAAKQ
[v1.13 ΔG (E211A/S216R)]
SEQ ID NO: 18
VAADIGAGLADALTAPLDHKDKGLQSLTLDQSVRKNEKLKLAAQGAEKTYGNGDSLNTGKLKNDKVSRFDFIRQIEV
DGKLITLESGEFQVYKQSHSALTALQTEQVQDSEDSGKMVAKRQFRIGDIAGEHTSFDKLPKGGSATYRGTAFGSD
DAGGKLTYTIDFAAKQGHGKIEHLKSPELNVELATAYIKPDEKRHAVISGSVLYNQDAKGSYRLGIFGGQAQEVAGS
AEVETANGIHHIGLAAKQ
[v1.15 mature polypeptide from strain NM452]
SEQ ID NO: 19
CSSGGGGSGGGGVAADIGAGLADALTAPLDHKDKGLKSLTLEDSISQNGTLTLSAQGAERTFKAGDKDNSLNTGKL
KNDKISRFDFIRQIEVDGQLITLESGEFQVYKQSHSALTALQTEQVQDSEHSGKMVAKRQFRIGDIVGEHTSFGKLP
KDVMATYRGTAFGSDDAGGKLTYTIDFAAKQGHGKIEHLKSPELNVDLAAADIKPDEKHHAVISGSVLYNQAEKGSY
SLGIFGGQAQEVAGSAEVETANGIRHIGLAAKQ
[v1.15 ΔG]
SEQ ID NO: 20
VAADIGAGLADALTAPLDHKDKGLKSLTLEDSISQNGTLTLSAQGAERTFKAGDKDNSLNTGKLKNDKISRFDFIRQI
EVDGQLITLESGEFQVYKQSHSALTALQTEQVQDSEHSGKMVAKRQFRIGDIVGEHTSFGKLPKDVMATYRGTAFG
SDDAGGKLTYTIDFAAKQGHGKIEHLKSPELNVDLAAADIKPDEKHHAVISGSVLYNQAEKGSYSLGIFGGQAQEVA
GSAEVETANGIRHIGLAAKQ
[v1.15 ΔG (S219R)]
SEQ ID NO: 21
VAADIGAGLADALTAPLDHKDKGLKSLTLEDSISQNGTLTLSAQGAERTFKAGDKDNSLNTGKLKNDKISRFDFIRQI
EVDGQLITLESGEFQVYKQSHSALTALQTEQVQDSEHSGKMVAKRQFRIGDIVGEHTSFGKLPKDVMATYRGTAFG
SDDAGGKLTYTIDFAAKQGHGKIEHLKSPELNVDLAAADIKPDEKHHAVISGSVLYNQAEKGSYRLGIFGGQAQEVA
GSAEVETANGIRHIGLAAKQ
[v1.15 ΔG (E214A/S219R)]
SEQ ID NO: 22
VAADIGAGLADALTAPLDHKDKGLKSLTLEDSISQNGTLTLSAQGAERTFKAGDKDNSLNTGKLKNDKISRFDFIRQI
EVDGQLITLESGEFQVYKQSHSALTALQTEQVQDSEHSGKMVAKRQFRIGDIVGEHTSFGKLPKDVMATYRGTAFG
SDDAGGKLTYTIDFAAKQGHGKIEHLKSPELNVDLAAADIKPDEKHHAVISGSVLYNQAAKGSYRLGIFGGQAQEVA
GSAEVETANGIRHIGLAAKQ
[v1.15 ΔG (E214A/E235A)]
SEQ ID NO: 23
VAADIGAGLADALTAPLDHKDKGLKSLTLEDSISQNGTLTLSAQGAERTFKAGDKDNSLNTGKLKNDKISRFDFIRQI
EVDGQLITLESGEFQVYKQSHSALTALQTEQVQDSEHSGKMVAKRQFRIGDIVGEHTSFGKLPKDVMATYRGTAFG
SDDAGGKLTYTIDFAAKQGHGKIEHLKSPELNVDLAAADIKPDEKHHAVISGSVLYNQAAKGSYSLGIFGGQAQEVA
GSAAVETANGIRHIGLAAKQ
[v2 wt from strain 2996]
SEQ ID NO: 24
MNRTAFCCLSLTAALILTACSSGGGGVAADIGAGLADALTAPLDHKDKSLQSLTLDQSVRKNEKLKLAAQGAEKTYG
NGDSLNTGKLKNDKVSRFDFIRQIEVDGQLITLESGEFQIYKQDHSAVVALQIEKINNPDKIDSLINQRSFLVSGLGG
EHTAFNQLPDGKAEYHGKAFSSDDAGGKLTYTIDFAAKQGHGKIEHLKTPEQNVELAAAELKADEKSHAVILGDTRY
GSEEKGTYHLALFGDRAQEIAGSATVKIGEKVHEIGIAGKQ
[v2 mature polypeptide]
SEQ ID NO: 25
CSSGGGGVAADIGAGLADALTAPLDHKDKSLQSLTLDQSVRKNEKLKLAAQGAEKTYGNGDSLNTGKLKNDKVSRF
DFIRQIEVDGQLITLESGEFQIYKQDHSAVVALQIEKINNPDKIDSLINQRSFLVSGLGGEHTAFNQLPDGKAEYHGK
AFSSDDAGGKLTYTIDFAAKQGHGKIEHLKTPEQNVELAAAELKADEKSHAVILGDTRYGSEEKGTYHLALFGDRAQ
EIAGSATVKIGEKVHEIGIAGKQ
[v2 ΔG]
SEQ ID NO: 26
VAADIGAGLADALTAPLDHKDKSLQSLTLDQSVRKNEKLKLAAQGAEKTYGNGDSLNTGKLKNDKVSRFDFIRQIEV
DGQLITLESGEFQIYKQDHSAVVALQIEKINNPDKIDSLINQRSFLVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQ
[v3 wt from strain M1239]
SEQ ID NO: 27
MNRTAFCCLSLTTALILTACSSGGGGSGGGGVAADIGTGLADALTAPLDHKDKGLKSLTLEDSIPQNGTLTLSAQGA
EKTFKAGDKDNSLNTGKLKNDKISRFDFVQKIEVDGQTITLASGEFQIYKQNHSAVVALQIEKINNPDKTDSLINQRS
FLVSGLGGEHTAFNQLPGGKAEYHGKAFSSDDPNGRLHYSIDFTKKQGYGRIEHLKTLEQNVELAAAELKADEKSHA
VILGDTRYGSEEKGTYHLALFGDRAQEIAGSATVKIGEKVHEIGIAGKQ
[v3 mature]
SEQ ID NO: 28
CSSGGGGSGGGGVAADIGTGLADALTAPLDHKDKGLKSLTLEDSIPQNGTLTLSAQGAEKTFKAGDKDNSLNTGKL
KNDKISRFDFVQKIEVDGQTITLASGEFQIYKQNHSAVVALQIEKINNPDKTDSLINQRSFLVSGLGGEHTAFNQLPG
GKAEYHGKAFSSDDPNGRLHYSIDFTKKQGYGRIEHLKTLEQNVELAAAELKADEKSHAVILGDTRYGSEEKGTYHL
ALFGDRAQEIAGSATVKIGEKVHEIGIAGKQ
[v3 ΔG]
SEQ ID NO: 29
VAADIGTGLADALTAPLDHKDKGLKSLTLEDSIPQNGTLTLSAQGAEKTFKAGDKDNSLNTGKLKNDKISRFDFVQKI
EVDGQTITLASGEFQIYKQNHSAVVALQIEKINNPDKTDSLINQRSFLVSGLGGEHTAFNQLPGGKAEYHGKAFSSD
DPNGRLHYSIDFTKKQGYGRIEHLKTLEQNVELAAAELKADEKSHAVILGDTRYGSEEKGTYHLALFGDRAQEIAGS
ATVKIGEKVHEIGIAGKQ
[v2 ΔG S32V/L123R]
SEQ ID NO: 30
VAADIGAGLADALTAPLDHKDKSLQSLTLDQVVRKNEKLKLAAQGAEKTYGNGDSLNTGKLKNDKVSRFDFIRQIEV
DGQLITLESGEFQIYKQDHSAVVALQIEKINNPDKIDSLINQRSFRVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQ
[v3 ΔG S32V/L126R]
SEQ ID NO: 31
VAADIGTGLADALTAPLDHKDKGLKSLTLEDVIPQNGTLTLSAQGAEKTFKAGDKDNSLNTGKLKNDKISRFDFVQK
IEVDGQTITLASGEFQIYKQNHSAVVALQIEKINNPDKTDSLINQRSFRVSGLGGEHTAFNQLPGGKAEYHGKAFSS
DDPNGRLHYSIDFTKKQGYGRIEHLKTLEQNVELAAAELKADEKSHAVILGDTRYGSEEKGTYHLALFGDRAQEIAG
SATVKIGEKVHEIGIAGKQ
[(23S_1.13_E211A/E232A)]
SEQ ID NO: 32
VAADIGAGLADALTAPLDHKDKSLQSLTLDQVVRKNEKLKLAAQGAEKTYGNGDSLNTGKLKNDKVSRFDFIRQIEV
DGQLITLESGEFQIYKQDHSAVVALQIEKINNPDKIDSLINQRSFRVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQGSGGGGVAADIGTGLADALTAPLDHKDKGLKSLTLEDVIPQNGTLTLSAQGAEKTFKAGD
KDNSLNTGKLKNDKISRFDFVQKIEVDGQTITLASGEFQIYKQNHSAVVALQIEKINNPDKTDSLINQRSFRVSGLGG
EHTAFNQLPGGKAEYHGKAFSSDDPNGRLHYSIDFTKKQGYGRIEHLKTLEQNVELAAAELKADEKSHAVILGDTRY
GSEEKGTYHLALFGDRAQEIAGSATVKIGEKVHEIGIAGKQGSGGGGVAADIGAGLADALTAPLDHKDKGLQSLTL
DQSVRKNEKLKLAAQGAEKTYGNGDSLNTGKLKNDKVSRFDFIRQIEVDGKLITLESGEFQVYKQSHSALTALQTEQ
VQDSEDSGKMVAKRQFRIGDIAGEHTSFDKLPKGGSATYRGTAFGSDDAGGKLTYTIDFAAKQGHGKIEHLKSPEL
NVELATAYIKPDEKRHAVISGSVLYNQDAKGSYSLGIFGGQAQEVAGSAAVETANGIHHIGLAAKQ
[23S_1.13_E211A/S216R]
SEQ ID NO: 33
VAADIGAGLADALTAPLDHKDKSLQSLTLDQVVRKNEKLKLAAQGAEKTYGNGDSLNTGKLKNDKVSRFDFIRQIEV
DGQLITLESGEFQIYKQDHSAVVALQIEKINNPDKIDSLINQRSFRVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQGSGGGGVAADIGTGLADALTAPLDHKDKGLKSLTLEDVIPQNGTLTLSAQGAEKTFKAGD
KDNSLNTGKLKNDKISRFDFVQKIEVDGQTITLASGEFQIYKQNHSAVVALQIEKINNPDKTDSLINQRSFRVSGLGG
EHTAFNQLPGGKAEYHGKAFSSDDPNGRLHYSIDFTKKQGYGRIEHLKTLEQNVELAAAELKADEKSHAVILGDTRY
GSEEKGTYHLALFGDRAQEIAGSATVKIGEKVHEIGIAGKQGSGGGGVAADIGAGLADALTAPLDHKDKGLQSLTL
DQSVRKNEKLKLAAQGAEKTYGNGDSLNTGKLKNDKVSRFDFIRQIEVDGKLITLESGEFQVYKQSHSALTALQTEQ
VQDSEDSGKMVAKRQFRIGDIAGEHTSFDKLPKGGSATYRGTAFGSDDAGGKLTYTIDFAAKQGHGKIEHLKSPEL
NVELATAYIKPDEKRHAVISGSVLYNQDAKGSYRLGIFGGQAQEVAGSAEVETANGIHHIGLAAKQ
[23S_1.15_S219R]
SEQ ID NO: 34
VAADIGAGLADALTAPLDHKDKSLQSLTLDQVVRKNEKLKLAAQGAEKTYGNGDSLNTGKLKNDKVSRFDFIRQIEV
DGQLITLESGEFQIYKQDHSAVVALQIEKINNPDKIDSLINQRSFRVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQGSGGGGVAADIGTGLADALTAPLDHKDKGLKSLTLEDVIPQNGTLTLSAQGAEKTFKAGD
KDNSLNTGKLKNDKISRFDFVQKIEVDGQTITLASGEFQIYKQNHSAVVALQIEKINNPDKTDSLINQRSFRVSGLGG
EHTAFNQLPGGKAEYHGKAFSSDDPNGRLHYSIDFTKKQGYGRIEHLKTLEQNVELAAAELKADEKSHAVILGDTRY
GSEEKGTYHLALFGDRAQEIAGSATVKIGEKVHEIGIAGKQGSGGGGVAADIGAGLADALTAPLDHKDKGLKSLTLE
DSISQNGTLTLSAQGAERTFKAGDKDNSLNTGKLKNDKISRFDFIRQIEVDGQLITLESGEFQVYKQSHSALTALQTE
QVQDSEHSGKMVAKRQFRIGDIVGEHTSFGKLPKDVMATYRGTAFGSDDAGGKLTYTIDFAAKQGHGKIEHLKSPE
LNVDLAAADIKPDEKHHAVISGSVLYNQAEKGSYRLGIFGGQAQEVAGSAEVETANGIRHIGLAAKQ
[23S_1.15_E214A/S219R]
SEQ ID NO: 35
VAADIGAGLADALTAPLDHKDKSLQSLTLDQVVRKNEKLKLAAQGAEKTYGNGDSLNTGKLKNDKVSRFDFIRQIEV
DGQLITLESGEFQIYKQDHSAVVALQIEKINNPDKIDSLINQRSFRVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQGSGGGGVAADIGTGLADALTAPLDHKDKGLKSLTLEDVIPQNGTLTLSAQGAEKTFKAGD
KDNSLNTGKLKNDKISRFDFVQKIEVDGQTITLASGEFQIYKQNHSAVVALQIEKINNPDKTDSLINQRSFRVSGLGG
EHTAFNQLPGGKAEYHGKAFSSDDPNGRLHYSIDFTKKQGYGRIEHLKTLEQNVELAAAELKADEKSHAVILGDTRY
GSEEKGTYHLALFGDRAQEIAGSATVKIGEKVHEIGIAGKQGSGGGGVAADIGAGLADALTAPLDHKDKGLKSLTLE
DSISQNGTLTLSAQGAERTFKAGDKDNSLNTGKLKNDKISRFDFIRQIEVDGQLITLESGEFQVYKQSHSALTALQTE
QVQDSEHSGKMVAKRQFRIGDIVGEHTSFGKLPKDVMATYRGTAFGSDDAGGKLTYTIDFAAKQGHGKIEHLKSPE
LNVDLAAADIKPDEKHHAVISGSVLYNQAAKGSYRLGIFGGQAQEVAGSAEVETANGIRHIGLAAKQ
[23S_1.15_E214A/E235A]
SEQ ID NO: 36
VAADIGAGLADALTAPLDHKDKSLQSLTLDQVVRKNEKLKLAAQGAEKTYGNGDSLNTGKLKNDKVSRFDFIRQIEV
DGQLITLESGEFQIYKQDHSAVVALQIEKINNPDKIDSLINQRSFRVSGLGGEHTAFNQLPDGKAEYHGKAFSSDDA
GGKLTYTIDFAAKQGHGKIEHLKTPEQNVELAAAELKADEKSHAVILGDTRYGSEEKGTYHLALFGDRAQEIAGSAT
VKIGEKVHEIGIAGKQGSGGGGVAADIGTGLADALTAPLDHKDKGLKSLTLEDVIPQNGTLTLSAQGAEKTFKAGD
KDNSLNTGKLKNDKISRFDFVQKIEVDGQTITLASGEFQIYKQNHSAVVALQIEKINNPDKTDSLINQRSFRVSGLGG
EHTAFNQLPGGKAEYHGKAFSSDDPNGRLHYSIDFTKKQGYGRIEHLKTLEQNVELAAAELKADEKSHAVILGDTRY
GSEEKGTYHLALFGDRAQEIAGSATVKIGEKVHEIGIAGKQGSGGGGVAADIGAGLADALTAPLDHKDKGLKSLTLE
DSISQNGTLTLSAQGAERTFKAGDKDNSLNTGKLKNDKISRFDFIRQIEVDGQLITLESGEFQVYKQSHSALTALQTE
QVQDSEHSGKMVAKRQFRIGDIVGEHTSFGKLPKDVMATYRGTAFGSDDAGGKLTYTIDFAAKQGHGKIEHLKSPE
LNVDLAAADIKPDEKHHAVISGSVLYNQAAKGSYSLGIFGGQAQEVAGSAAVETANGIRHIGLAAKQ
[v1.1 ΔG + His tag]
SEQ ID NO: 37
VAADIGAGLADALTAPLDHKDKGLQSLTLDQSVRKNEKLKLAAQGAEKTYGNGDSLNTGKLKNDKVSRFDFIRQIEV
DGQLITLESGEFQVYKQSHSALTAFQTEQIQDSEHSGKMVAKRQFRIGDIAGEHTSFDKLPEGGRATYRGTAFGSD
DAGGKLTYTIDFAAKQGNGKIEHLKSPELGLAAKQLNVDLAAADIKPDGKRHAVISGSVLYNQAEKGSYSLGIFGGKA
QEVAGSAEVKTVNGIRHLEHHHHHH
[v1.13 ΔG + His tag]
SEQ ID NO: 38
VAADIGAGLADALTAPLDHKDKGLQSLTLDQSVRKNEKLKLAAQGAEKTYGNGDSLNTGKLKNDKVSRFDFIRQIEV
DGKLITLESGEFQVYKQSHSALTALQTEQVQDSEDSGKMVAKRQFRIGDIAGEHTSFDKLPKGGSATYRGTAFGSD
DAGGKLTYTIDFAAKQGHGKIEHLKSPELNVELATAYIKPDEKRHAVISGSVLYNQDEKGSYSLGIFGGQAQEVAGSA
EVETANGIHHIGLAAKQLEHHHHHH
[v1.13 ΔG (E211A)]
SEQ ID NO: 39
VAADIGAGLADALTAPLDHKDKGLQSLTLDQSVRKNEKLKLAAQGAEKTYGNGDSLNTGKLKNDKVSRFDFIRQIEV
DGKLITLESGEFQVYKQSHSALTALQTEQVQDSEDSGKMVAKRQFRIGDIAGEHTSFDKLPKGGSATYRGTAFGSD
DAGGKLTYTIDFAAKQGHGKIEHLKSPELNVELATAYIKPDEKRHAVISGSVLYNQDAKGSYSLGIFGGQAQEVAGS
AEVETANGIHHIGLAAKQLEHHHHHH
[v1.13 ΔG (S216R)]
SEQ ID NO: 40
VAADIGAGLADALTAPLDHKDKGLQSLTLDQSVRKNEKLKLAAQGAEKTYGNGDSLNTGKLKNDKVSRFDFIRQIEV
DGKLITLESGEFQVYKQSHSALTALQTEQVQDSEDSGKMVAKRQFRIGDIAGEHTSFDKLPKGGSATYRGTAFGSD
DAGGKLTYTIDFAAKQGHGKIEHLKSPELNVELATAYIKPDEKRHAVISGSVLYNQDEKGSYRLGIFGGQAQEVAGS
AEVETANGIHHIGLAAKQLEHHHHHH
[v1.15 ΔG + His tag]
SEQ ID NO: 41
VAADIGAGLADALTAPLDHKDKGLKSLTLEDSISQNGTLTLSAQGAERTFKAGDKDNSLNTGKLKNDKISRFDFIRQI
EVDGQLITLESGEFQVYKQSHSALTALQTEQVQDSEHSGKMVAKRQFRIGDIVGEHTSFGKLPKDVMATYRGTAFG
SDDAGGKLTYTIDFAAKQGHGKIEHLKSPELNVDLAAADIKPDEKHHAVISGSVLYNQAEKGSYSLGIFGGQAQEVA
GSAEVETANGIRHIGLAAKQLEHHHHHH
[v1.15 ΔG (E214A) + His tag]
SEQ ID NO: 42
VAADIGAGLADALTAPLDHKDKGLKSLTLEDSISQNGTLTLSAQGAERTFKAGDKDNSLNTGKLKNDKISRFDFIRQI
EVDGQLITLESGEFQVYKQSHSALTALQTEQVQDSEHSGKMVAKRQFRIGDIVGEHTSFGKLPKDVMATYRGTAFG
SDDAGGKLTYTIDFAAKQGHGKIEHLKSPELNVDLAAADIKPDEKHHAVISGSVLYNQAAKGSYSLGIFGGQAQEVA
GSAEVETANGIRHIGLAAKQLEHHHHHH
[fHbp 231 wt fusion polypeptide]
SEQ ID NO: 43
VAADIGAGLADALTAPLDHKDKSLQSLTLDQSVRKNEKLKLAAQGAEKTYGNGDSLNTGKLKNDKVSFDFIRQIEVD
GQLITLESGEFQIYKQDHSAVVALQIEKINNPDKIDSLINQRSFLVSGLGGEHTAFNQLPDGKAEYHGKAFSSDDAG
GKLTYTIDFAAKQGHGKIEHLKTPEQNVELAAAELKADEKSHAVILGDTRYGSEEKGTYHLALFGDRAQEIAGSATVK
IGEKVHEIGIAGKQGSGGGGVAADIGTGLADALTAPLDHKDKGLKSLTLEDSIPQNGTLTLSAQGAEKTFKAGDKDN
SLNTGKLKNDKISRFDFVQKIEVDGQTITLASGEFQIYKQNHSAVVALQIEKINNPDKTDSLINQRSFLVSGLGGEHT
AFNQLPGGKAEYHGKAFSSDDPNGRLHYSIDFTKKQGYGRIEHLKTLEQNVELAAAELKADEKSHAVILGDTRYGSE
EKGTYHLALFGDRAQEIAGSATVKIGEKVHEIGIAGKQGSGGGGVAADIGAGLADALTAPLDHKDKGLQSLTLDQSV
RKNEKLKLAAQGAEKTYGNGSLNTGKLKNDKVSRFDFIRQIEVDGQLITLESGEFQVYKQSHSALTAFQTEQIQDSE
HSGKMVAKRQFRIGDIAGEHTSFDKLPEGGRATYRGTAFGSDDAGGKLTYTIDFAAKQGNGKIEHLKSPELNVDLAA
AIKPDGKRHAVISGSVLYNQAEKGSYSLGIFGKAQEVAGSAEVKTVNGIRHIGLAAKQ
[fHbp 231S fusion polypeptide]
SEQ ID NO: 44
VAADIGAGLADALTAPLDHKDKSLQSLTLDQVVRKNEKLKLAAQGAEKTYGNGDSLNTGKLKNDKVSFDFIRQIEVD
GQLITLESGEFQIYKQDHSAVVALQIEKINNPDKIDSLINQRSFRVSGLGGEHTAFNQLPDGKAEYHGKAFSSDDAG
GKLTYTIDFAAKQGHGKIEHLKTPEQNVELAAAELKADEKSHAVILGDTRYGSEEKGTYHLALFGDRAQEIAGSATVK
IGEKVHEIGIAGKQGSGGGGVAADIGTGLADALTAPLDHKDKGLKSLTLEDVIPQNGTLTLSAQGAEKTFKAGDKDN
SLNTGKLKNDKISRFDFVQKIEVDGQTITLASGEFQIYKQNHSAVVALQIEKINNPDKTDSLINQRSFRVSGLGGEHT
AFNQLPGGKAEYHGKAFSSDDPNGRLHYSIDFTKKQGYGRIEHLKTLEQNVELAAAELKADEKSHAVILGDTRYGSE
EKGTYHLALFGDRAQEIAGSATVKIGEKVHEIGIAGKQGSGGGGVAADIGAGLADALTAPLDHKDKGLQSLTLDQSV
SKNEKLKLAAQGAEKTYGNGSLNTGKLKNDKVSRFDFIRQIEVDGQLITLESGEFQVYKQSHSALTAFQTEQIQDSE
HSGKMVAKRQFRIGDIAGEHTSFDKLPEGGRATYRGTAFGSDDAGGKLTYTIDFAAKQGNGKIEHLKSPELNVDLAA
AIKPDGKRHAVISGSVLYNQAEKGSYSLGIFGKAQEVAGSAEVKTVNGIRHIGLAAKQ
[v1.13 full-length wt sequence]
SEQ ID NO: 45
MNRTAFCCFSLTAALILTACSSGGGGVAADIGAGLADALTAPLDHKDKGLQSLTLDQSVRKNEKLKLAAQGAEKTYG
NGDSLNTGKLKNDKVSRFDFIRQIEVDGKLITLESGEFQVYKQSHSALTALQTEQVQDSEDSGKMVAKRQFRIGDIA
GEHTSFDKLPKGGSATYRGTAFGSDDAGGKLTYTIDFAAKQGHGKIEHLKSPELNVELATAYIKPDEKRHAVISGSVL
YNQDEKGSYSLGIFGGQAQEVAGSAEVETANGIHHIGLAAKQ
[v1.15 full-length wt sequence]
SEQ ID NO: 46
MNRTTFCCLSLTAALILTACSSGGGGSGGGGVAADIGAGLADALTAPLDHKDKGLKSLTLEDSISQNGTLTLSAQGA
ERTFKAGDKDNSLNTGKLKNDKISRFDFIRQIEVDGQLITLESGEFQVYKQSHSALTALQTEQVQDSEHSGKMVAKR
QFRIGDIVGEHTSFGKLPKDVMATYRGTAFGSDDAGGKLTYTIDFAAKQGHGKIEHLKSPELNVDLAAADIKPDEKH
HAVISGSVLYNQAEKGSYSLGIFGGQAQEVAGSAEVETANGIRHIGLAAKQ
[mature fHbp v1.1]
SEQ ID NO: 47
CSSGGGGVAADIGAGLADALTAPLDHKDKGLQSLTLDQSVRKNEKLKLAAQGAEKTYGNGDSLNTGKLKNDKVSRF
DFIQIEVDGQLITLESGEFQVYKQSHSALTAFQTEQIQDSEHSGKMVAKRQFRIGDIAGEHTSFDKLPEGGRATYRG
TAFGSDDAGGKLTYTIDFAAKQGNGKIEHLKSPELNVDLAAADIKPDGKRHAVISGSVLYNQAEKGSYSLGIFGGKA
QEVAGSAEVKTVNGIRHIGLAAKQ
[optional N-terminal amino acid sequence]
SEQ ID NO: 48
MGPDSDRLQQRR
[SEQ ID NO: 48 + SEQ ID NO: 33; 23S_1.13_E211A/S216R with additional
N-terminal amino acid sequence]
SEQ ID NO: 49
MGPDSDRLQQRRVAADIGAGLADALTAPLDHKDKSLQSLTLDQVVRKNEKLKLAAQGAEKTYGNGDSLNTGKLKN
DKVSRFDFIRQIEVDGQLITLESGEFQIYKQDHSAVVALQIEKINNPDKIDSLINQRSFRVSGLGGEHTAFNQLPDGK
AEYHGKAFSSDDAGGKLTYTIDFAAKQGHGKIEHLKTPEQNVELAAAELKADEKSHAVILGDTRYGSEEKGTYHLAL
FGDRAQEIAGSATVKIGEKVHEIGIAGKQGSGGGGVAADIGTGLADALTAPLDHKDKGLKSLTLEDVIPQNGTLTLS
AQGAEKTFKAGDKDNSLNTGKLKNDKISRFDFVQKIEVDGQTITLASGEFQIYKQNHSAVVALQIEKINNPDKTDSLI
NQRSFRVSGLGGEHTAFNQLPGGKAEYHGKAFSSDDPNGRLHYSIDFTKKQGYGRIEHLKTLEQNVELAAAELKAD
EKSHAVILGDTRYGSEEKGTYHLALFGDRAQEIAGSATVKIGEKVHEIGIAGKQGSGGGGVAADIGAGLADALTAPL
DHKDKGLQSLTLDQSVRKNEKLKLAAQGAEKTYGNGDSLNTGKLKNDKVSRFDFIRQIEVDGKLITLESGEFQVYKQ
SHSALTALQTEQVQDSEDSGKMVAKRQFRIGDIAGEHTSFDKLPKGGSATYRGTAFGSDDAGGKLTYTIDFAAKQG
HGKIEHLKSPELNVELATAYIKPDEKRHAVISGSVLYNQDAKGSYRLGIFGGQAQEVAGSAEVETANGIHHIGLAAK
Q
[linker]
SEQ ID NO: 50
GGSG
[linker]
SEQ ID NO: 51
GGSGG
[linker]
SEQ ID NO: 52
GSGSG
[linker]
SEQ ID NO: 53
GSGGG
[linker]
SEQ ID NO: 54
GGGSG
[linker]
SEQ ID NO: 55
GSSSG
[linker]
SEQ ID NO: 56
GSGGGG

### MODES FOR CARRYING OUT THE INVENTION

The invention will now be further defined by reference to the following non-limiting examples.

### EXAMPLES

### Example 1: Lyophilization of MenACWY Antigens Composition—Excipient Selection

Lyophilization allows obtaining the title vaccine components in a stable form over long-term storage, suitable for reconstitution with an aqueous solution containing other vaccine's components prior to administration.

For the lyophilization process of MenACWY antigens composition, addition of the bulking agent was tested to evaluate the potential impact of excipient addition on the lyophilization process and on the composition itself.

As shown below, a surprising result was obtained by adding an amount of bulking agent, in particular sucrose, to the pre-lyophilization bulk formulation that was much reduced with respect to inventors' experience with similar vaccines.

The reduced amount of bulking agent below 6% w/v in the pre-lyo bulk solution, together with an appropriate phosphate buffering agent, as detailed below, provided good results in terms of antigen aggregation and of residual moisture % in the lyophilized product, verified under accelerated stability conditions.

To this end, 3 freeze-dried products have been prepared differing from one another only for the sucrose concentration in the pre-lyo bulk formulation of MenACWY antigens, namely at 2%, 3% and 5% by weight with respect to the total volume of the formulation.

A 10 mM phosphate buffer concentration was applied to the 3 tested pre-lyo bulk formulations, whose composition was as summarized below in Table 1.

TABLE 1
Composition of MenACWY antigens pre-lyo bulk formulation

| Component | Concentration in pre-lyo bulk formulations | | |
|---|---|---|---|
| MenA-CRM₁₉₇ (μg/ml) | 24 | 24 | 24 |
| MenC-CRM₁₉₇ (μg/ml) | 12 | 12 | 12 |
| MenW-CRM₁₉₇ (μg/ml) | 12 | 12 | 12 |
| MenY-CRM₁₉₇ (μg/ml) | 12 | 12 | 12 |
| Sucrose (% w/v) | 2 | 3 | 5 |
| K₂HPO₄ (mM) | 10 | 10 | 10 |

The pH of the 3 formulations was adjusted to a final pH value of 7.2 by adding KOH into the buffers.

All 3 batches were freeze-dried within the same lyophilization cycle, stored at 2-8° C. and then incubated at 37° C. and at 25° C. with 60% RH approximately 3 months after the production day.

RM % and aggregation % were determined in the lyophilized product at time zero and in stability, up to 2 months and to 6 months at 37° C. and at 25° C./60% RH, respectively. The Karl Fisher method was used to determine RM % in the lyophilized cakes with a colorimetric Karl Fisher Titrator with Oven Metrohm 831 under the following conditions:
Oven temperature: 110° C.
Nitrogen flow rate: 60 ml/min
Stop drift: 10 g/min

All lyophilized products, produced from pre-lyo bulks formulations at different sucrose concentrations, were within the 3% RM specification limit up to the end of both accelerated stability tests at the 2 different temperatures 37° C. and 25° C., with a similar absolute water content between the 3 different formulations.

Aggregation % was determined by SE-UPLC, and the results obtained are summarized in the following Table 2. No significant variation in the aggregation percentages was observed amongst the three different formulations tested and, also under stability conditions, no increasing aggregation trend was observed over time for the pre-lyo formulations of this invention.

TABLE 2
Aggregation % under accelerated stabilities conditions on freeze-dried
products from pre-lyo formulation with different sucrose concentration

| | Sucrose | | | | | | |
|---|---|---|---|---|---|---|---|
| | concentration | Time points | | | | | |
| Stability | in pre-lyo | Time | 2 | 4 | 2 | 3 | 6 |
| conditions | bulk (% w/v) | zero | weeks | weeks | months | months | months |
| 37° C. | 2 | 4.6 | 4.5 | 4.8 | 4.2 | | |
| | 3 | 5.1 | 3.2 | 5.1 | 3.6 | | |
| | 5 | 5.9 | 6.1 | 6.2 | 4.7 | | |
| 25° C./ | 2 | 4.6 | | 2.8 | | 3.8 | 4.3 |
| 60% RH | 3 | 5.1 | | 3.6 | | 4.0 | 5.1 |
| | 5 | 5.9 | | 4.4 | | 5.1 | 5.0 |

### Example 2: Preparation of MenB Antigens Composition in Liquid Formulation Including fHbp231.13—Preformulation Studies and Excipient Selection

Preliminary formulation studies have been carried out for a MenB antigens composition of the invention, including the BEXSERO antigens, to which a KPi buffer simulating the presence of fHbp231.13 was also added at three different concentrations, 100 μg/ml, 200 μg/ml, and 400 μg/ml, in order to verify the behavior of the new antigens composition, when formulated with excipients and buffers and added with Alum as adsorbing agent for the protein antigens as detailed below.

In these experiments, the formulations have been stored at 2-8° C. and at 25° C. for up to 3 months, then verifying the percentage of adsorption of MenB antigens onto Alum at defined time points by RP-UPLC.

The results were surprising in particular in that the fHbp-GNA2091 antigen of the BEXSERO formulation increases its adsorption onto Alum at all tested temperatures, particularly at higher temperature of 25° C. In fact, in the BEXSERO composition the fHbp-GNA2091 antigen is not completely adsorbed onto Alum and the addition of another antigen with further phosphate buffer to the MenB composition was expected to further decrease adsorption because of a possible competitive effect amongst the adsorbed proteins onto Alum. This experiment demonstrated that the addition of further phosphate buffer linked to the addition of a further antigen fHbp231.13, to obtain a heptavalent composition with seven MenB antigens, has no detrimental effect on the overall antigens' absorption onto Alum.

Moreover, by simultaneous variation of formulation factors by using a DoE (i.e. Design of Experiments) approach, different concentrations of buffers/excipients were evaluated for the MenB antigens liquid formulation of this invention, including aluminum hydroxide at 3 mg/ml, OMV antigen at 50 μg/ml and other protein antigens at 100 μg/ml, as summarized in the Table 3 below:

TABLE 3
Buffers/excipients evaluated with related concentrations

| Component | Minimum value | Medium point | Maximum value |
|---|---|---|---|
| Histidine pH | 6.1 | 6.5 | 6.9 |
| Sucrose (% w/v) | 1.4 | 1.7 | 2.0 |
| NaCl (mg/ml) | 2.80 | 4.50 | 6.25 |

The experimental results have showed that the NaCl concentration had a significant effect on the amount of fHbp-GNA2091 antigen adsorbed on Alum: the antigen is better adsorbed at lower concentrations of NaCl, in particular at the lowest concentration tested of 2.8 mg/ml it was observed the higher amount of antigen adsorbed at all different pH values tested. Sucrose concentration had a less significant impact on the adsorption of the antigen onto Alum with respect to the NaCl concentration.

In order to optimize the excipients amounts the following formulations in Table 4 have been prepared and tested with the maximum amount of the fHbp231.13 antigen:

TABLE 4

| | | | | | Other MenB |
|---|---|---|---|---|---|
| Formulation | NaCl | Sucrose | His | fHbp231.13 | antigens |
| No. | [mg/ml] | [% w/v] | pH | [μg/ml] | [μg/ml] |
| 2 | 2.8 | 2 | 6.3 | 400 | 50 for OMV, |
| 4 | 3.8 | 2 | | 400 | 100 for |
| 6 | 2.8 | 3 | | 400 | other Ags |
| 8 | 3.8 | 3 | | 400 | |
| 10 | 6.25 | 2 | | 400 | |
| BEXSERO | 6.25 | 2 | | n/a | |

The tests carried out on these formulations have shown better results in the terms of good adsorption of all MenB antigens onto Alum for the formulations Nos. 2 and 6 having the lower concentration of NaCl, with the formulation No. 6 having a concentration of sucrose of 3% w/v which is the optimal one in terms of stability of the drug substance for the fHbp231.13 antigen.

The percentage of adsorption onto Alum of the MenB antigens was analyzed by RP-UPLC for each of the antigens according to the following method. 1 ml of the sample was centrifuged at 2100×g for 20 minutes at 20° C.; 500 μl of supernatant were taken and mixed with 15 μl of a solution of zwittergent in water.

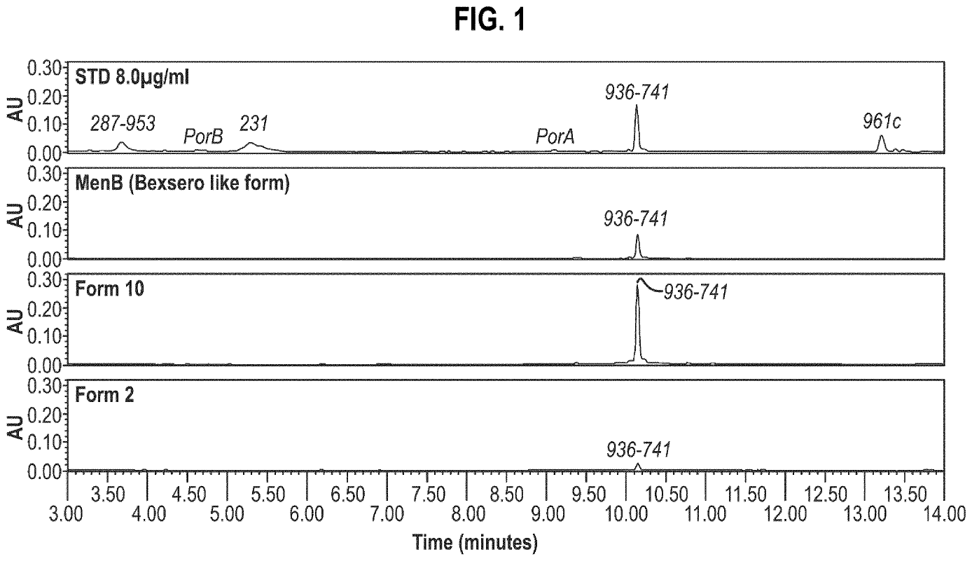

Samples were injected in duplicate in UPLC system to record the chromatogram. In FIG. 1 there is a chromatogram illustrated with overlapping profiles for formulations 2, 10 and BEXSERO in comparison with a standard point at the concentration of 8.0 μg/ml.

For all formulations only the peak of fHbp-GNA2091 is detected in the chromatographic profiles, showing that this is the only antigen not completely adsorbed onto Alum and that the addition of fHbp231.13 polypeptide in the formulation of the invention does not promote therefore the desorption of the other MenB components. Furthermore, from the chromatogram in FIG. 1 it is evident that a lower concentration of NaCl (formulation No. 2) does assist adsorption of fHbp-GNA2091, showing a lower amount of non-adsorbed antigen with respect to other formulation with higher NaCl concentration (formulation No. 10, also indicated herein as BEXSERO-like). For the avoidance of doubt, in the present specification by the expression “BEXSERO-like formulation” is meant a formulation of the MenB antigens of the present invention but with a concentration of NaCl of 6.25 mg/ml, which is the same of the BEXSERO formulation and higher than the NaCl concentration in the excipients of the formulation of this invention.

A similar trend of absorption was observed after reconstitution of a MenACWY lyo composition of this invention with a 2% or 3% sucrose content. Also, same results described above were observed for formulations containing lower amounts of the fHbp231.13 antigen, in particular 100 μg/ml and 200 μg/ml.

During this study the impact of the buffers used to prepare the liquid formulation of Men antigens was also evaluated, finding that the pH value of the histidine buffer has an impact on the pH of the final formulations of MenB antigens as they are and after reconstitution in the final vaccine composition. With the target of a final pH around 6.5, histidine buffer at pH from 6.1 to 6.3 was found as the most appropriate to combine with fHbp231.13 in KPi at pH 7.2.

During this study different concentrations of Alum were tested, e.g. 2.8 mg/ml and 3.0 mg/ml, and a concentration of 3.0 mg/ml was found better and chosen for further experiments.

Antigenicity evaluation—On the following formulations A, B and D summarized in Table 5:

TABLE 5

| | | | | | | Other MenB |
|---|---|---|---|---|---|---|
| Formulation | NaCl | Sucrose | pH | fHbp231.13 | Alum | antigens |
| No. | [mg/ml] | [% w/v] | Histidine | [μg/ml] | [mg/ml] | [μg/ml] |
| A | 2.80 | 3 | 6.1 | 400 | 3.0 | 50 for OMV, |
| B | 2.80 | 3 | 6.3 | 100 | 3.0 | 100 for other |
| D | 6.25 | 2 | 6.3 | n/a | 3.0 | Ags |

A Flow Cytometry analysis was carried out for the detection of antigens adsorbed on the surface of Alum suspensions allowing the monitoring of their conformational behavior over time: antigens adsorbed on Alum can be recognized by a specific monoclonal antibody and then revealed by a secondary labeled antibody. According to signal intensity and polydispersity, information about antigen conformational behavior and orientation on Alum was obtained.

8 different mAbs specific for each MenB antigen in the formulation of this invention were used to prove the presence of critical epitopes of the antigens when formulated according to this invention in the form adsorbed onto Alum.

In the following Table 6 the mAbs used for the FACS analysis are listed together with their main characteristics:

TABLE 6

| | | | | | Recognition | | | |
|---|---|---|---|---|---|---|---|---|
| | | | | | of denatured | Bactericidal | Clinical | |
| Ag | mAb | Subclass | Affinity | Epitope | antigen | activity | relevance | Supplier |
| 287 | 42A4A2 | IgG1 | Medium | Conformational | Binding not | No | Yes | Areta |
| | | | | (located in the | completely | | | |
| | | | | C-terminal | abolished | | | |
| | | | | region. more | at 80° C. | | | |
| | | | | stable - high | | | | |
| | | | | Tm. ≈ 95° C.) | | | | |
| 287 | 31E10/E7 | IgG1 | High | Linear (located | Binding not | No | Yes | Areta |
| | | | | in the N- | completely | | | |
| | | | | terminal | abolished | | | |
| | | | | region. less | at 80° C. | | | |
| | | | | stable - low | | | | |
| | | | | Tm. ≈ 60° C.) | | | | |
| 287 | 10E8 | IgG1 | High | Conformational | Not | Yes | Not | Takis |
| | | | | | available | | determined | |
| 936-741 | 1201D7 | IgG2b | High | Conformational | No | Yes | Yes | Areta |
| 741_v231.13 | 131 | IgG2b | High | Recognizes | Not | Yes | Not | GSK |
| | | | | var 3 and var 2 | determined | | determined | Belgium |
| 961c | 9F11 | IgG1 | High | Conformational | No | Yes | Yes | Areta |
| | | | | Recognizes wt | | | | |
| | | | | and shifted | | | | |
| | | | | form. does not | | | | |
| | | | | recognize | | | | |
| | | | | truncated | | | | |
| | | | | forms | | | | |
| 961c | 6E3 | IgG1 | High | Conformational | No | Yes | Yes | Areta |
| | | | | Recognizes wt. | | | | |
| | | | | shifted form. | | | | |
| | | | | and truncated | | | | |
| | | | | forms | | | | |
| OMVnz | PorA 1.4 | IgG2a | N/A | Conformational | Not | Yes | Yes | NIBSC |
| | | | | (VR2 loop) | determined | | | Cat |
| | | | | | | | | 02/148 |

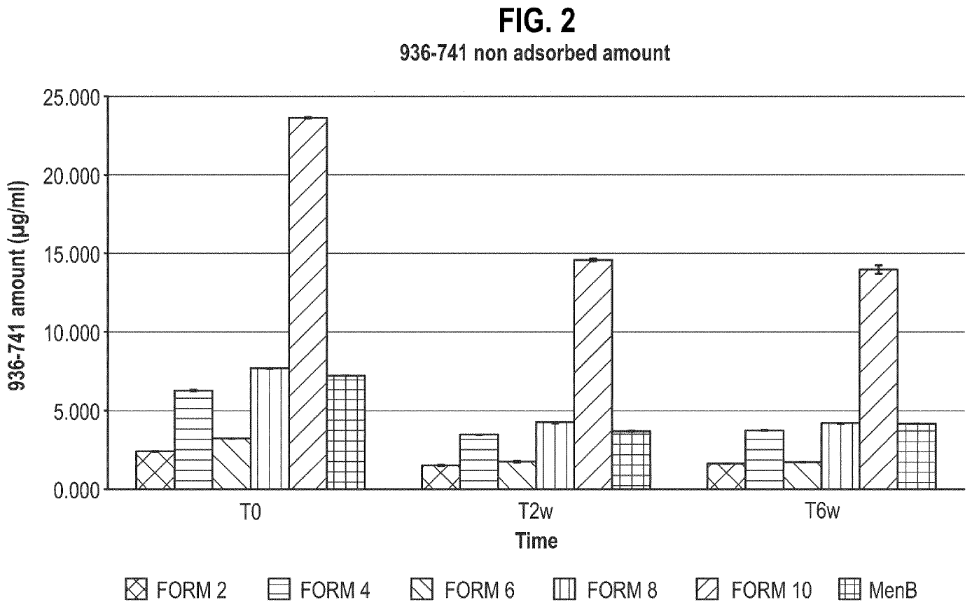

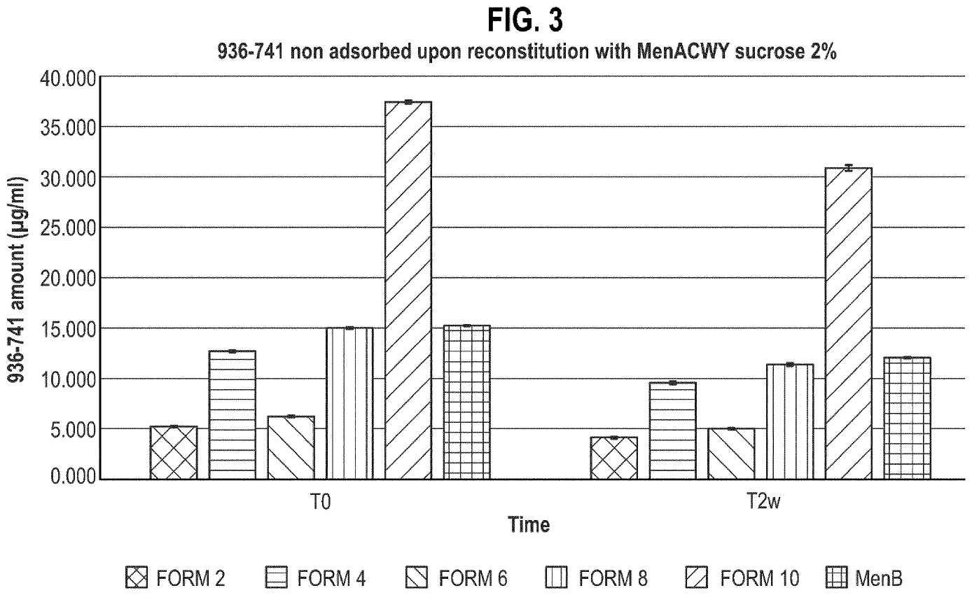

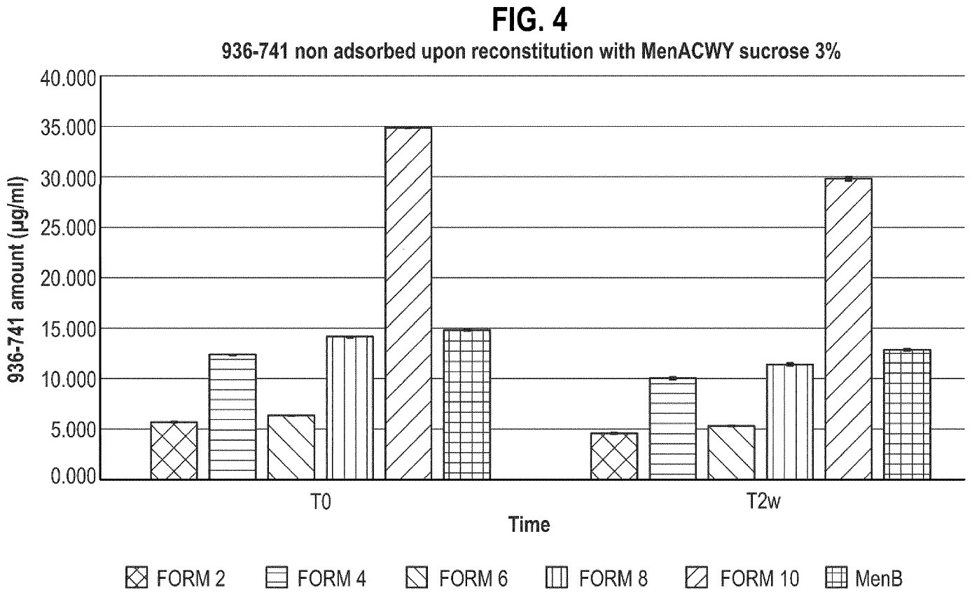

Results for the present formulations A and B and for the reference formulation D are illustrated in FIGS. 2, 3 and 4 respectively. They prove that the antigenicity was maintained in the present formulations and show that all mAbs recognize the respective antigens similarly for all formulations when antigens are present at the same concentrations.

### Example 4: Preparation of MenACWY Antigens Lyophilized Drug Product and of MenB Antigens Liquid Component

Based on the results showed above, the following compositions have been prepared, having the respective components illustrated in Tables 7 and 8 below.

TABLE 7
Composition of MenACWY antigens composition
as a lyophilized product

| Component | Quantity per dose (0.5 ml) |
|---|---|
| MenA-CRM₁₉₇ | 10 μg MenA saccharide |
| MenC-CRM₁₉₇ | 5 μg MenC saccharide |
| MenW-CRM₁₉₇ | 5 μg MenW saccharide |
| MenY-CRM₁₉₇ | 5 μg MenY saccharide |
| Sucrose | 12.5 mg |
| KH₂PO₄ + K₂HPO₄ | 0.40 mg |

TABLE 8
Composition of Men B antigens composition in
liquid formulation (filling volume 0.64 ml)

| Component | Concentration | |
|---|---|---|
| NHBA-GNA1030 | 100 | μg/ml |
| fHbp-GNA2091 | 100 | μg/ml |
| NadA | 100 | μg/ml |
| OMV | 50 | μg/ml |
| fHbp231.13 | 100 | μg/ml |
| NaCl | 2.8 | mg/ml |
| Sucrose | 3% | w/v |
| Histidine | 10 | mM |
| Aluminum hydroxide | 3.0 | mg/ml |

### Example 5: Reconstitution of Lyophilized MenACWY Antigens Composition with Liquid MenB Antigens Composition

The composition of the reconstituted vaccine of MenACWY antigens lyophilized drug product with MenB antigens liquid component, for each dose to be delivered corresponding to a volume of 0.5 ml, is shown in the following Table 9:

TABLE 9
Composition per dose (0.5 ml) of reconstituted vaccine of MenACWY
antigens lyo drug product with MenB antigens liquid formulation

| Component | Quantity per dose (0.5 ml) | |
|---|---|---|
| MenA saccharide | 10 | μg |
| MenC saccharide | 5 | μg |
| MenW saccharide | 5 | μg |
| MenY saccharide | 5 | μg |
| rp287-953 | 50 | μg |
| rp936-741 | 50 | μg |
| rp961c | 50 | μg |
| OMV | 25 | μg |
| fHbp231.13 | 50-100-200 | μg¹ |
| NaCl | 1.4 | mg |
| Sucrose | 27.5 | mg² |
| Potassium phosphate salts | 0.54 mg to 0.90 mg³ | |
| Histidine | 0.776 | mg |
| Aluminum hydroxide | 1.54 | mg⁴ |

| | |
|---|---|
| Water for injection | to obtain 0.5 ml dose |

¹According to the dose.
²Sum of the contributions of sucrose derived from lyophilized MenACWY (2.5% w/v) and that in the heptavalent composition of MenB antigens (3% w/v).
³Potassium phosphate salts calculated according to the sum of the contribution of phosphate coming from lyophilized MenACWY and from the heptavalent MenB composition drug substances, also considering the three doses reported for fHbp231.13.
⁴Aluminum hydroxide corresponding to 0.5 mg of aluminum element.

Appropriate procedures have been defined in order to guarantee that the lyophilized product is reconstituted with the required volume of liquid formulation to obtain the target dosage of 0.5 ml for injection.

Reconstitution and injection procedures have been designed in order to guarantee the dose injection for all the configurations defined for the target dose, using the materials summarized in the following Table 10.

The procedures imply the use of a same single needle both for the reconstitution/withdrawing step and for the injection to the subject.

TABLE 10
Materials used for the reconstitution procedures

| | Total quantity |
|---|---|
| | for both |
| | reconstitution |
| Material | and injection |
| Pre-filled syringe containing MenB liquid formulation | 1 |
| Vial containing lyophilized MenACWY | 1 |
| Plunger rod | 1 |
| 23G × 1″ sterile disposable needle for | 1 |
| reconstitution and injection | |

Standard sanitary practices for contamination prevention during reconstitution and withdrawing procedures need to be followed in order to prevent product contamination at use.

On the reconstituted vaccine composition, that was an opalescent white suspension, stability tests were conducted in particular to verify adsorption onto Alum of MenB protein antigens after reconstitution. Studies by Reverse-Phase-UPLC have been carried out to estimate the amount of not adsorbed MenB antigens, in particular of fHbp-GNA2091, on Alum over time, as described in detail above. In the following Table 11 are summarized the amounts (g/ml) of fHbp-GNA2091 not adsorbed on aluminum hydroxide before and after reconstitution with the MenACWY lyophilized drug product for the formulations indicated, whose compositions are reported in the Table 4 above.

TABLE 11
Quantification of not adsorbed fHbp-GNA2091 onto Alum

| | not adsorbed fHbp-GNA2091 (μg/ml) | | |
|---|---|---|---|
| | | after | after |
| | | reconstitution | reconstitution |
| Formulation | before | with MenACWY | with MenACWY |
| No. | reconstitution | 2% sucrose | 3% sucrose |
| Form. 2 | 2.412 | 5.252 | 5.659 |
| Form 4 | 6.283 | 12.707 | 12.366 |
| Form 6 | 3.222 | 6.239 | 6.355 |
| Form 8 | 7.702 | 15.011 | 14.141 |
| Form 10 | 23.627 | 37.421 | 34.785 |
| BEXSERO | 7.225 | 15.253 | 14.821 |

At time 0 the non-adsorbed fHbp-GNA2091 protein antigen was >10% for formulations BEXSERO-like (Form 10) containing higher amounts of NaCl in the liquid formulation of MenB antigens, but this amount decreased over time up to 3 months, in particular for samples at 25° C. Moreover, for the liquid formulations of MenB antigens with a lower amount of NaCl (Form 2, 4, 6, 8) the amount of non-adsorbed fHbp-GNA2091 protein antigen was in any case lower than 10%, taken as a target limit to ensure an optimal antigens integrity and immunogenicity.

Upon reconstitution with the lyophilized MenACWY product, the amount of not adsorbed MenB antigen under evaluation increased for all tested formulations but substantially in the same way irrespective of the sucrose quantity tested, amounting to 2 or 3%.

FIG. 5 show, in the form of histograms, the results obtained for all formulations tested before reconstitution at time 0 and after storage at 2-8° C. for 2 weeks and 6 weeks. FIGS. 6 and 7 show, in the form of histograms, the results collected over time for all the formulations upon reconstitution at time 0 and after 2 weeks at 2-8° C. Reported data represent the average of 3 replicates. From these FIGS. 6 and 7 it is clear that the percentage of not adsorbed fHbp-GNA2091 antigen is higher than 10% at all time points evaluated for the formulation Form 10, while the formulations containing a lower amount of NaCl are those in which the same antigen is better adsorbed onto Alum.

### CONCLUSIONS

As showed by the results in the experimental part above, the liquid formulation of the Men B antigens composition guarantees that all relevant quality requirements of the final vaccine are met, in particular in terms of pH and osmolality of the vaccine composition, integrity and immunogenicity of each and all antigens.

The fHbp231.13 polypeptide added to the MenB antigens of the BEXSERO composition proved to be stable, compatible with other antigens in the composition, when it is added as adsorbing agent in the liquid formulation of MenB antigens: an optimal percentage of adsorption onto Alum of all antigens is observed for the MenB antigens liquid formulation and also for the reconstituted vaccine composition containing all MenABCWY antigens.

1. An immunogenic composition against N. meningitidis serogroup B, comprising: a meningococcal NHBA antigen, a meningococcal NadA antigen, a meningococcal fHbp antigen, a meningococcal outer membrane vesicles (OMVs), and a fusion polypeptide of meningococcal fHbp polypeptides, wherein said composition is a liquid formulation comprising an adsorbing agent for said antigens and polypeptides and one or more pharmaceutically acceptable tonicity modifying agents.

2. The immunogenic composition of claim 1, wherein said tonicity modifying agents are selected from sodium chloride, sorbitol, sucrose and mixtures thereof.

3. The immunogenic composition of claim 1, wherein said liquid formulation comprises an aqueous solution of sodium chloride and sucrose.

4. The immunogenic composition of claim 1, wherein said liquid formulation has a concentration of sodium chloride 3.8 mg/ml.

5. The immunogenic composition of claim 1, wherein said liquid formulation has a concentration of sodium chloride comprised between 1 mg/ml and 3.5 mg/ml, e.g. 2.8 mg/ml.

6. (canceled)

7. The immunogenic composition of claim 1, wherein said liquid formulation has a concentration of sucrose comprised between 2.5 and 3.5% w/v, e.g. 3% w/v.

8. The immunogenic composition of claim 1, wherein said fusion polypeptide of meningococcal fHbp polypeptides comprises v1, v2 and v3 meningococcal fHbp polypeptides in the order v2-v3-v1 from N- to C-terminus.

9. The immunogenic composition of claim 8, wherein the v1 meningococcal fHbp polypeptide is a mutant v1.13 fHbp polypeptide, having an amino acid sequence differing from SEQ ID NO: 16 by at least one or more of the substitutions E211A, S216R and E232A.

10. The immunogenic composition of claim 1, wherein said meningococcal NHBA antigen and said meningococcal fHbp antigen are fusion proteins with meningococcal accessory proteins, e.g. a NHBA-GNA1030 fusion protein and a GNA2091-fHbp fusion protein.

11. The immunogenic composition of claim 1, wherein said adsorbing agent comprises a compound containing aluminium selected from aluminium hydroxide (Alum), aluminium salts and mixtures thereof, preferably is Alum.

12. (canceled)

13. The immunogenic composition of claim 1, wherein said OMVs are present at 50 μg/ml, each of said NHBA antigen, NadA antigen, and fHbp antigen are present at 100 μg/ml, said fusion polypeptide is present at 100 or 200 μg/ml, sucrose is present at 2.5 to 3.5% w/v, e.g. 3% w/v, a buffer is prepared at pH 6.1-6.3, Alum is present at 2 to 5 mg/ml, e.g. 3 mg/ml and sodium chloride is present at 2.0 to 3.5 mg/ml, e.g. 2.8 mg/ml.

14. A reconstituted vaccine composition comprising an immunogenic composition against N. meningitidis serogroups A, C, W135, and Y, comprising capsular saccharides antigens of the meningococcal serogroups A, C, W135, and Y that are conjugated to carrier protein(s) in a solid form (e.g. freeze-dried), reconstituted with the immunogenic composition against N. meningitidis serogroup B liquid formulation as defined in claim 1.

15. The reconstituted vaccine of claim 14, wherein said immunogenic composition against N. meningitidis serogroups A, C, W135, and Y comprises a bulking agent, selected from the group consisting of sucrose, mannitol, trehalose and mixtures thereof; preferably is sucrose.

16. (canceled)

17. The reconstituted vaccine of claim 14, further comprising a buffering agent that is a phosphate buffer comprising potassium monobasic phosphate, to which potassium dibasic phosphate is added.

18. The reconstituted vaccine of claim 14, wherein said capsular saccharides antigens of the meningococcal serogroups A, C, W135, and Y are conjugated to CRM197 as carrier protein.

19. The reconstituted vaccine of claim 14, comprising sodium chloride at a concentration of 2 to 3.5 mg/ml (e.g. 2.8 mg/ml).

20. A kit comprising (i) a first container (e.g. a prefilled syringe) comprising the immunogenic composition against N. meningitidis serogroup B liquid formulation of claim 1; and (ii) a second container (e.g. a vial) comprising an immunogenic composition against N. meningitidis serogroups A, C, W135, and Y, comprising capsular saccharides antigens of the meningococcal serogroups A, C, W135, and Y that are conjugated to carrier protein(s) in a solid form, wherein said first container and said second container are containers separated from each other or together form a multi-container.

21. (canceled)

22. (canceled)

23. (canceled)

24. A method for preparing the reconstituted vaccine composition comprising an immunogenic composition against N. meningitidis serogroups A, B, C, W135, and Y of claim 14, comprising a step of reconstituting the immunogenic composition against N. meningitidis serogroups A, C, W135, and Y in solid form, with the immunogenic composition against N. meningitidis serogroup B liquid formulation as defined in claim 1.

25. A method for the treatment or prevention of a meningococcal infection or disease caused by N. meningitidis in a subject in need thereof comprising administering to said subject a therapeutically effective amount of the immunogenic composition according to claim 1.

26. A method of inducing an immune response to N. meningitidis in a subject, the method comprising administering a therapeutically or prophylactically effective amount of the immunogenic composition according to claim 1.

27. (canceled)
